(19) United States
(12) Patent Application Publication
(10) Pub. No.: US 2014/0243273 A1
(43) Pub. Date: Aug. 28, 2014

(54) METHODS FOR TREATING INFLAMMATORY DISEASES AND PHARMACEUTICAL COMBINATIONS USEFUL THEREFOR

(71) Applicant: Vertex Pharmaceuticals Incorporated, Boston, MA (US)

(72) Inventors: Irina Nikolaevna Kadiyala, Newton, MA (US); Shahla Jamzad, Belmont, MA (US); Thomas Carl Hoock, Westford, MA (US); Lori Kell Taylor, Boston, MA (US); Kathryn Lea Sewell, Brighton, MA (US)

(73) Assignee: Vertex Pharmaceuticals Incorporated, Boston, MA (US)

(21) Appl. No.: 14/271,589

(22) Filed: May 7, 2014

Related U.S. Application Data
(63) Continuation of application No. PCT/US2012/063712, filed on Nov. 6, 2012.
(60) Provisional application No. 61/556,666, filed on Nov. 7, 2011.
(60) Provisional application No. 61/594,818, filed on Feb. 3, 2012.
(60) Provisional application No. 61/636,024, filed on Apr. 20, 2012.

(51) Int. Cl. A61K 31/53; A61K 31/506; A61K 45/06; C07D 471/04

(52) U.S. Cl. CPC A61K 31/53; C07D 471/04; A61K 31/506; A61K 45/06
USPC 514/16.6; 514/256; 514/245; 514/249; 514/263.2; 514/16.7; 514/19.9; 514/110; 514/263.3

(57) ABSTRACT

The present invention provides method of treating or lessening the severity of a disease selected from spondyloarthropathy, systemic lupus erythematosus, rheumatoid arthritis, or any combination thereof comprising the administration of a compound of Formula I and an optional co-therapy (e.g., chemotherapy agent, DMARD, or any combination thereof). The present invention also provides a pharmaceutical composition comprising a compound of Formula I, a method of manufacturing a pharmaceutical composition comprising a compound of Formula I, and a method of administering a pharmaceutical composition comprising a solid form of a compound of Formula I.

### CROSS REFERENCE TO RELATED APPLICATION

The present PCT application claims the benefit of U.S. application Ser. No. 61/556,666, filed on Nov. 7, 2011; U.S. application Ser. No. 61/594,818, filed on Feb. 3, 2012; and U.S. application Ser. No. 61/636,024, filed on Apr. 20, 2012. Each of these applications is hereby incorporated by reference in its entirety.

### FIELD OF THE INVENTION

The present invention relates to pharmaceutical compositions and methods for treating diseases or conditions selected from spondyloarthropathy, systemic lupus erythematosus, rheumatoid arthritis, or any combination thereof with a compound of Formula I or a combination of a compound of Formula I and a chemotherapy agent (e.g., methotrexate).

### BACKGROUND

The Janus kinases (JAK) are a family of tyrosine kinases consisting of JAK1, JAK2, JAK3, and TYK2. The JAKs play a critical role in cytokine signaling. The down-stream substrates of the JAK family of kinases include the signal transducer and activator of transcription (STAT) proteins. JAK/STAT signaling has been implicated in the mediation of many abnormal immune responses such as SLE, RA, and spondyloarthropathies including peripheral spondyloarthropathy, axial spondyloarthropathy, UC, Crohn's disease, ankylosing spondylitis, reactive arthritis, Reiter's syndrome, and psoriatic arthritis. Moreover, JAK kinases represent an established therapeutic target for these diseases. For example, JAK kinases are an established therapeutic target for treating SLE, RA, spondyloarthropathies including peripheral spondyloarthropathy, axial spondyloarthropathy, UC, Crohn's disease, ankylosing spondylitis, reactive arthritis, Reiter's syndrome, and psoriatic arthritis. Stump K. L., et al., Arthritis Res. Ther. (2011) 13:R68; Fridman J. S., et al., J Immunol. (2010) 184:5298-5307; West K., Curr. Op. Investig. Drugs (2009) 10:491-504; Kremer J. M. et al., Arthritis Rheumatism (2009) 60(7):1895-1905; Xiong, W. et al., Ther Adv Musculoskelet Dis. (2011) 3(5): 255-266; Panes, J. et al. 19^{th} Ann. Eur. Gastroenterology Week (Oct. 22-26, 2011) Stockholm, S E, P1456; and Drugs in R & D “Tofacitinib” (2010) 10(4):271-84.

Compounds described as kinase inhibitors, particularly the JAK family kinases, are disclosed in WO 2005/095400 and WO 2007/084557. Also disclosed in these publications are processes and intermediates for preparing these compounds. There remains, however, a need for stable bioavailable pharmaceutical compositions useful for treating patients suffering from abnormal immune responses such as spondyloarthropathy (e.g., peripheral spondyloarthropathy, axial spondyloarthropathy, reactive arthritis, Reiter's syndrome, psoriatic arthritis, ankylosing spondylitis, ulcerative colitis, Crohn's disease, or any combination thereof), SLE, RA, or any combination thereof and methods of administering the same.

### SUMMARY OF THE INVENTION

In general, the invention relates to pharmaceutical compositions and methods for treating or lessening the severity of a disease or disorder selected from SLE, RA, spondyloarthropathy, or any combination thereof with a compound of Formula I or a combination of a compound of Formula I and a chemotherapy agent (e.g., methotrexate).

One aspect of the present invention provides a method for treating or lessening the severity of a disease selected from spondyloarthropathy (e.g., peripheral spondyloarthropathy, axial spondyloarthropathy, reactive arthritis, Reiter's syndrome, psoriatic arthritis, ankylosing spondylitis, ulcerative colitis, Crohn's disease, or any combination thereof), rheumatoid arthritis (RA), systemic lupus erythematosus (SLE), or any combination thereof comprising administering to a patient in need thereof a chemotherapy agent and a compound of Formula I

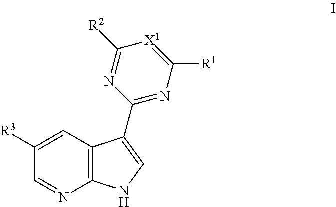

or a pharmaceutically acceptable salt thereof, wherein:

X¹ is N or CR⁴;

R² is H or halo;

R³ is H or halo;

R⁴ is H or halo;

R¹ is

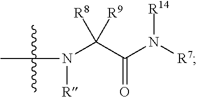

R″ is H or an unsubstituted C₁₋₂ aliphatic;

R⁸ is an unsubstituted C₁₋₄ aliphatic;

R⁹ is an unsubstituted C₁₋₄ aliphatic;

R⁷ is a C₁₋₃ aliphatic optionally substituted with up to 3 occurrences of F; and

R¹⁴ is H or unsubstituted C₁₋₂ alkyl.

In some embodiments, the chemotherapy agent comprises methotrexate, azathioprine (e.g., Imuran), cyclosporine, cyclophosphamide (e.g., Cytoxan), 6-mercaptopurine, or any combination thereof. And, in some instances, the chemotherapy agent comprises an injectable formulation or an oral formulation.

In other embodiments, the patient is administered from about 5 mg to about 100 mg of the chemotherapy agent per month.

In some embodiments, for the compound of Formula I, R² is H or F.

In some embodiments, R³ is H or Cl.

In some embodiments, each of R⁸ and R⁹ is independently selected from methyl, ethyl, propyl, iso-propyl, butyl, or tert-butyl. For instance, each of R⁸ and R⁹ is independently selected from methyl or ethyl.

In some embodiments, R¹⁴ is H or methyl.

In some embodiments, R⁷ is an unsubstituted C₁₋₃ aliphatic.

In some embodiments, R⁷ is a C₁₋₃ aliphatic substituted with 1-3 occurrences of F.

In some embodiments, R⁷ is a group selected from —CH₂CH₃, —CH₂CF₃, —CH₂CH₂CH₃, or —CHCH₃CH₃.

And, in other embodiments, the compound of Formula I is selected from the compounds in Table 1, provided below.

In some embodiments, the compound of Formula I is administered at least once per day (e.g., from 1 to 4 times per day). In other embodiments, the compound of Formula I is administered at least twice per day.

In some embodiments, the compound of Formula I is orally administered to the patient.

In some embodiments, at least about 25 mg of the compound of Formula I is administered to the patient once per day. In some embodiments, at least about 50 mg of the compound of Formula I is administered to the patient once per day. In some embodiments, at least about 100 mg of the compound of Formula I is administered to the patient once per day. For example, at least about 150 mg of the compound of Formula I is administered to the patient once per day. In other examples, at least about 200 mg of the compound of Formula I is administered to the patient once per day.

In other embodiments, at least about 25 mg of the compound of Formula I is administered to the patient twice per day. In other embodiments, at least about 50 mg of the compound of Formula I is administered to the patient twice per day. In other embodiments, at least about 100 mg of the compound of Formula I is administered to the patient twice per day.

Another aspect of the present invention provides a method for treating or lessening the severity of a disease selected from systemic lupus erythematosus, ulcerative colitis, Crohn's disease, ankylosing spondylitis, peripheral spondyloathropathy, axial spondyloathropathy, reactive arthritis, Reiter's syndrome, psoriatic arthritis, or any combination thereof comprising administering to a patient in need thereof a compound of Formula I

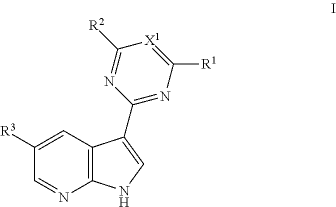

or a pharmaceutically acceptable salt thereof, wherein:

X¹ is N or CR⁴;

R² is H or halo;

R³ is H or halo;

R⁴ is H or halo;

R¹ is

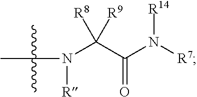

R″ is H or an unsubstituted C₁₋₂ aliphatic;

R⁸ is an unsubstituted C₁₋₄ aliphatic;

R⁹ is an unsubstituted C₁₋₄ aliphatic;

R⁷ is a C₁₋₃ aliphatic optionally substituted with up to 3 occurrences of F; and

R¹⁴ is H or unsubstituted C₁₋₂ alkyl.

Some embodiments further comprise administering one or more DMARDs (e.g., adalimumab, leflunomide, sulfasalazine, infliximab, minocycline, rituximab, golimumab, or any combination thereof) to the patient.

Some embodiments further comprise administering a chemotherapy agent to the patient. And, in some examples, the chemotherapy agent comprises methotrexate, azathioprine (e.g., Imuran), cyclosporine, cyclophosphamide (e.g., Cytoxan), 6-mercaptopurine, or any combination thereof.

In some embodiments, the chemotherapy agent comprises an injectable formulation or an oral formulation.

In some embodiments, the patient is administered from about 5 mg to about 100 mg of the chemotherapy agent per month.

In some embodiments, for the compound of Formula I, R² is H or F.

In some embodiments, R³ is H or Cl.

In some embodiments, each of R⁸ and R⁹ is independently selected from methyl, ethyl, propyl, iso-propyl, butyl, or tert-butyl. For example, each of R⁸ and R⁹ is independently selected from methyl or ethyl.

In some embodiments, R¹⁴ is H or methyl.

In some embodiments, R⁷ is an unsubstituted C₁₋₃ aliphatic.

In some embodiments, R⁷ is a C₁₋₃ aliphatic substituted with 1-3 occurrences of F.

In some embodiments, R⁷ is a group selected from —CH₂CH₃, —CH₂CF₃, —CH₂CH₂CH₃, or —CHCH₃CH₃.

In some embodiments, the compound of Formula I is selected from the compounds in Table 1.

In some embodiments, the compound of Formula I is administered at least once per day (e.g., from 1 to 4 times per day). For example, the compound of Formula I is administered at least twice per day.

In some embodiments, the compound of Formula I is orally administered to the patient in need thereof.

In some embodiments, at least about 50 mg of the compound of Formula I is administered to the patient once per day.

In some embodiments, at least about 750 mg of the compound of Formula I is administered to the patient once per day.

In some embodiments, at least about 100 mg of the compound of Formula I is administered to the patient once per day.

In some embodiments, at least about 150 mg of the compound of Formula I is administered to the patient once per day.

In some embodiments, at least about 200 mg of the compound of Formula I is administered to the patient once per day.

In some embodiments, at least about 100 mg of the compound of Formula I is administered to the patient twice per day.

Another aspect of the present invention provides a method for treating or lessening the severity of a disease selected from spondyloarthropathy (e.g., peripheral spondyloarthropathy, axial spondyloarthropathy, reactive arthritis, Reiter's syndrome, psoriatic arthritis, ankylosing spondylitis, ulcerative colitis, Crohn's disease, or any combination thereof), systemic lupus erythematosus, rheumatoid arthritis (RA), or any combination thereof comprising administering to a patient in need thereof a chemotherapy agent and a pharmaceutical composition comprising a compound of Formula I

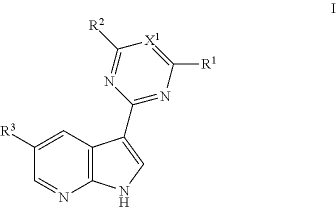

or a pharmaceutically acceptable salt thereof, wherein:

X¹ is N or CR⁴;

R² is H or halo;

R³ is H or halo;

R⁴ is H or halo;

R¹ is

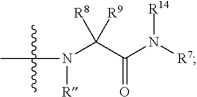

R″ is H or an unsubstituted C₁₋₂ aliphatic;

R⁸ is an unsubstituted C₁₋₄ aliphatic;

R⁹ is an unsubstituted C₁₋₄ aliphatic;

R⁷ is a C₁₋₃ aliphatic optionally substituted with up to 3 occurrences of F; and

R¹⁴ is H or unsubstituted C₁₋₂ alkyl.

In some embodiments, the chemotherapy agent comprises methotrexate, azathioprine (e.g., Imuran), cyclosporine, cyclophosphamide (e.g., Cytoxan), 6-mercaptopurine, or any combination thereof.

In some embodiments, the chemotherapy agent comprises an injectable formulation or an oral formulation.

In some embodiments, the patient is administered from about 5 mg to about 100 mg of the chemotherapy agent per month.

In some embodiments, for the compound of Formula I, R² is H or F.

In some embodiments, R³ is H or Cl.

In some embodiments, each of R⁸ and R⁹ is independently selected from methyl, ethyl, propyl, iso-propyl, butyl, or tert-butyl. For example, each of R⁸ and R⁹ is independently selected from methyl or ethyl.

In some embodiments, R¹⁴ is H or methyl.

In some embodiments, R⁷ is an unsubstituted C₁₋₃ aliphatic.

In some embodiments, R⁷ is a C₁₋₃ aliphatic substituted with 1-3 occurrences of F.

In some embodiments, R⁷ is a group selected from —CH₂CH₃, —CH₂CF₃, —CH₂CH₂CH₃, or —CHCH₃CH₃.

In some embodiments, the compound of Formula I is selected from the compounds in Table 1.

In some embodiments, the pharmaceutical composition further comprises a tablet. And, in some of these embodiments, the tablet further comprises a diluent, a binder, a glidant, a disintegrant, a surfactant, a lubricant, or any combination thereof.

In some embodiments, the tablet is administered at least once per day (e.g., from 1 to 4 times per day).

In some embodiments, the tablet comprises at least about 10 mg (e.g., from about 25 mg to about 250 mg) of the compound of Formula I.

In some embodiments, the tablet comprises from about 15 mg to about 100 mg of the compound of Formula I.

In some embodiments, the tablet is administered at least twice per day.

Some embodiments further comprise administering once per day at least one tablet comprising the pharmaceutical composition.

Some embodiments further comprise administering twice per day at least one tablet comprising the pharmaceutical composition.

In some embodiments, each tablet further comprises from about 5 mg to about 100 mg of the compound of Formula I.

Another aspect of the present invention provides a method for treating or lessening the severity of a disease selected from spondyloarthropathy (e.g., peripheral spondyloarthropathy, axial spondyloarthropathy, reactive arthritis, Reiter's syndrome, psoriatic arthritis, ankylosing spondylitis, ulcerative colitis, Crohn's disease, or any combination thereof), systemic lupus erythematosus, rheumatoid arthritis (RA), or any combination thereof comprising administering to a patient in need thereof a compound of Formula I

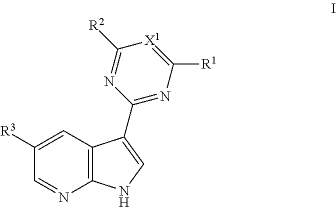

or a pharmaceutically acceptable salt thereof, wherein:

X¹ is N or CR⁴;

R² is H or halo;

R³ is H or halo;

R⁴ is H or halo;

R¹ is

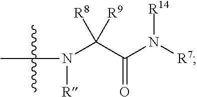

R″ is H or an unsubstituted C₁₋₂ aliphatic;

R⁸ is an unsubstituted C₁₋₄ aliphatic;

R⁹ is an unsubstituted C₁₋₄ aliphatic;

R⁷ is a C₁₋₃ aliphatic optionally substituted with up to 3 occurrences of F; and

R¹⁴ is H or unsubstituted C₁₋₂ alkyl,

wherein:

at least about 100 mg of the compound of Formula I is administered to the patient at least once per day (e.g., from 1 to 4 times per day).

In some embodiments, about 100 mg of the compound of formula I is administered to the patient once per day.

In some embodiments, about 150 mg of the compound of formula I is administered to the patient once per day.

In some embodiments, about 200 mg of the compound of formula I is administered to the patient once per day.

In some embodiments, about 100 mg of the compound of formula I is administered to the patient twice per day.

Some embodiments further comprise administering to the patient a chemotherapy agent.

In some embodiments, for the compound of Formula I, R² is H or F.

In some embodiments, R³ is H or Cl.

In some embodiments, each of R⁸ and R⁹ is independently selected from methyl, ethyl, propyl, iso-propyl, butyl, or tert-butyl. For example, each of R⁸ and R⁹ is independently selected from methyl or ethyl.

In some embodiments, R¹⁴ is H or methyl.

In some embodiments, R⁷ is an unsubstituted C₁₋₃ aliphatic.

In some embodiments, R⁷ is a C₁₋₃ aliphatic substituted with 1-3 occurrences of F.

In some embodiments, R⁷ is a group selected from —CH₂CH₃, —CH₂CF₃, —CH₂CH₂CH₃, or —CHCH₃CH₃.

In some embodiments, the compound of Formula I is selected from the compounds in Table 1.

Another aspect of the present invention provides a pharmaceutical composition comprising:

a. a compound of Formula I

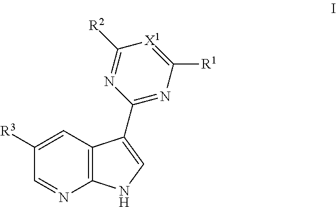

or a pharmaceutically acceptable salt thereof, wherein:

X¹ is N or CR⁴;

R² is H or halo;

R⁴ is H or halo;

R¹ is

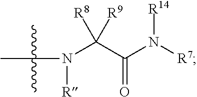

R″ is H or an unsubstituted C₁₋₂ aliphatic;

R⁸ is an unsubstituted C₁₋₄ aliphatic;

R⁹ is an unsubstituted C₁₋₄ aliphatic;

R⁷ is a C₁₋₃ aliphatic optionally substituted with up to 3 occurrences of F; and

R¹⁴ is H or unsubstituted C₁₋₂ alkyl; and

one or more excipients comprising a diluent, a disintegrant, a wetting agent, a binder, a glidant, a lubricant, or any combination thereon, wherein the compound of Formula I has a concentration of from about 25 wt % to about 60 wt % by weight of the composition, and the total concentration for the one or more excipients is from about 40 wt % to about 75 wt % by weight of the composition.

In some embodiments, X¹ is N, CH, or CF.

In some embodiments, R″ is H or methyl.

In some embodiments, R² is H or F.

In some embodiments, each of R⁸ and R⁹ is independently selected from methyl, ethyl, propyl, iso-propyl, butyl, or tert-butyl. For example, each of R⁸ and R⁹ is independently selected from methyl or ethyl.

In some embodiments, R¹⁴ is H or methyl.

In some embodiments, R⁷ is an unsubstituted C₁₋₃ aliphatic.

In some embodiments, R⁷ is a C₁₋₃ aliphatic substituted with 1-3 occurrences of F.

In some embodiments, R⁷ is a group selected from —CH₂CH₃, —CH₂CF₃, —CH₂CH₂CH₃, or —CHCH₃CH₃.

In some embodiments, the compound of Formula I is selected from the compounds in Table 1.

In some embodiments, the pharmaceutical composition further comprises a diluent, and the diluent comprises lactose, sorbitol, cellulose, calcium phosphate, starch, sugar, or any combination thereof. For example, the diluent comprises lactose and has a concentration of about 10 wt % or greater by weight of the composition.

In some embodiments, the pharmaceutical composition further comprises a disintegrant, and the disintegrant comprises sodium croscarmellose, sodium starch glycolate, or any combination thereof. For example, the disintegrant comprises sodium croscarmellose and has a concentration of about 10 wt % or less by weight of the composition.

In some embodiments, the pharmaceutical composition further comprises a wetting agent, and the wetting agent comprises sodium lauryl sulfate, sodium stearyl fumarate, polyoxyethylene 20 sorbitan mono-oleate, or any combination thereof. For example, the wetting agent comprises sodium lauryl sulfate and has a concentration of about 10 wt % or less by weight of the composition.

In some embodiments, the pharmaceutical composition further comprises a binder, and the binder comprises microcrystalline cellulose, dibasic calcium phosphate, sucrose, corn starch, modified cellulose, or any combination thereof. For example, the binder comprises microcrystalline cellulose and has a concentration of at least about 1 wt % by weight of the composition.

In some embodiments, the pharmaceutical composition further comprises a glidant, and the glidant comprises colloidal silicon dioxide, talc, or any combination thereof. For example, the glidant comprises colloidal silicon dioxide and has a concentration of about 2 wt % or less by weight of the composition.

In some embodiments, the pharmaceutical composition further comprises a lubricant, and the lubricant comprises magnesium stearate, stearic acid, hydrogenated oil, sodium stearyl fumarate, or any combination thereof. For example, the lubricant comprises magnesium stearate and has a concentration of less than about 2 wt % by weight of the composition.

In some embodiments, the pharmaceutical composition further comprises about 25 wt % to about 35 wt % of the compound of Formula I by weight of the composition. For example, the composition comprises from about 45 wt % to about 55 wt % of the compound of Formula I by weight of the composition.

In some embodiments, the pharmaceutical composition comprises a tablet.

### BRIEF DESCRIPTION OF THE DRAWINGS

The following figures are provided by way of example and are not intended to limit the scope of the claimed invention.

FIGS. 1A-1E are plots of body weight loss as a function of time for control and test groups of mice described in Example 8.

FIG. 2 is a bar graph showing colon length for groups of test mice described in Example 8.

FIG. 3 is a bar graph showing colon weight for groups of test mice described in Example 8.

FIG. 4 is a bar graph showing colon weight per unit of colon length for groups of test mice described in Example 8.

FIGS. 5A-5E are photographs of cross-sections of colons for a representative mouse for each of the groups of test mice described in Example 8.

FIG. 6 is a bar graph showing the histology score for groups of test mice described in Example 8.

FIG. 7 is a bar graph showing scores for individual parameters of three groups of test mice described in Example 8.

FIG. 8 is a bar graph showing the concentration of IFN-γ in colon tissue in groups of test mice described in Example 8.

FIG. 9 is a bar graph showing the concentration of IL-17 in colon tissue in groups of test mice described in Example 8.

FIG. 10 is a bar graph showing the concentration of IL-8 in colon tissue in groups of test mice described in Example 8.

FIG. 11 is a bar graph showing the concentration of MCP-1 in colon tissue in groups of test mice described in Example 8.

FIG. 12 is a bar graph showing the concentration of IL-1β in colon tissue in groups of test mice described in Example 8.

FIG. 13 is a bar graph showing the concentration of IL-6 in colon tissue in groups of test mice described in Example 8.

FIG. 14 is a bar graph showing the concentration of TNF-a in colon tissue in groups of test mice described in Example 8.

FIG. 15 is a bar graph showing the concentration of IL-12p40 in colon tissue in groups of test mice described in Example 8.

FIG. 16 is a bar graph showing the concentration of IL-10 in colon tissue in groups of test mice described in Example 8.

FIG. 17 is a bar graph showing the concentration of IL-13 in colon tissue in groups of test mice described in Example 8.

FIG. 18 is a plot of the frequency of splenic CD4⁺αβ-TCR⁺cells in groups of test mice described in Example 8.

### DETAILED DESCRIPTION

The present invention provides methods of treating or lessening the severity of a disease or disorder selected from spondyloarthropathy (e.g., peripheral spondyloarthropathy, axial spondyloarthropathy, reactive arthritis, Reiter's syndrome, psoriatic arthritis, ankylosing spondylitis, ulcerative colitis, Crohn's disease, or any combination thereof), systemic lupus erythematosus, rheumatoid arthritis (RA), or any combination thereof comprising the administration of a compound of Formula I.

### I. DEFINITIONS

As used herein, the term “active pharmaceutical ingredient” or “API” refers to a biologically active compound. An exemplary API include a protein kinase inhibitor (e.g., a JAK inhibitor) such as a compound of Formula I:

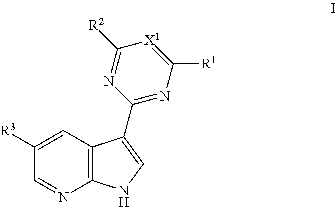

or a pharmaceutically acceptable salt thereof, wherein:

X¹ is N or CR⁴;

R² is H or halo;

R³ is H or halo;

R⁴ is H or halo;

R¹ is

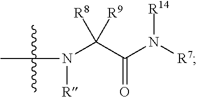

R″ is H or an unsubstituted C₁₋₂ aliphatic;

R⁸ is an unsubstituted C₁₋₄ aliphatic;

R⁹ is an unsubstituted C₁₋₄ aliphatic;

R⁷ is a C₁₋₃ aliphatic optionally substituted with up to 3 occurrences of F; and

R¹⁴ is H or unsubstituted C₁₋₂ alkyl.

As used herein, a “protein kinase inhibitor” refers to a compound that exhibits biological activity characterized by blocking the action of one or more protein kinases.

As used herein, “spondyloarthropathy” refers to any joint disease of the vertebral column including spondylarthritis. Examples of spondyloarthropathies include reactive arthritis, Reiter's syndrome, psoriatic arthritis, ankylosing spondylitis, ulcerative colitis, Crohn's disease, and any combination thereof.

As used herein, an “excipient” is an inactive ingredient in a pharmaceutical composition. Examples of excipients include fillers or diluents, wetting agents (e.g., surfactants), binders, glidants, lubricants, disintegrants, or the like.

As used herein, a “disintegrant” is an excipient that hydrates a pharmaceutical composition and aids in tablet dispersion. Examples of disintegrants include sodium croscarmellose and/or sodium starch glycolate.

As used herein, a “diluent” or “filler” is an excipient that adds bulkiness to a pharmaceutical composition. Examples of fillers include lactose, sorbitol, celluloses, calcium phosphates, starches, sugars (e.g., mannitol, sucrose, or the like) or any combination thereof.

As used herein, a “wetting agent” or a “surfactant” is an excipient that imparts pharmaceutical compositions with enhanced solubility and/or wetability. Examples of wetting agents include sodium lauryl sulfate (SLS), sodium stearyl fumarate (SSF), polyoxyethylene 20 sorbitan mono-oleate (e.g., Tween™), or any combination thereof.

As used herein, a “binder” is an excipient that imparts a pharmaceutical composition with enhanced cohesion or tensile strength (e.g., hardness). Examples of binders include dibasic calcium phosphate, sucrose, corn (maize) starch, microcrystalline cellulose, and modified cellulose (e.g., hydroxymethyl cellulose).

As used herein, a “glidant” is an excipient that imparts a pharmaceutical compositions with enhanced flow properties. Examples of glidants include colloidal silica and/or talc.

As used herein, a “colorant” is an excipient that imparts a pharmaceutical composition with a desired color. Examples of colorants include commercially available pigments such as FD&C Blue #1 Aluminum Lake, FD&C Blue #2, other FD&C Blue colors, titanium dioxide, iron oxide, and/or combinations thereof. Other colorants include commercially available pigments such as FD&C Green #3.

As used herein, a “lubricant” is an excipient that is added to pharmaceutical compositions that are pressed into tablets. The lubricant aids in compaction of granules into tablets and ejection of a tablet of a pharmaceutical composition from a die press. Examples of lubricants include magnesium stearate, stearic acid (stearin), hydrogenated oil, sodium stearyl fumarate, or any combination thereof.

As used herein, “friability” refers to the property of a tablet to remain intact and withhold its form despite an external force of pressure. Friability can be quantified using the mathematical expression presented in equation 1:

$$\% friability = 100 \times \frac{( {W}_{0} - {W}_{f} )}{{W}_{0}} \qquad ( 1 )$$

wherein W₀ is the original weight of the tablet and W_{f} is the final weight of the tablet after it is put through the friabilator.

Friability is measured using a standard USP testing apparatus that tumbles experimental tablets for 100 revolutions. Some tablets of the present invention have a friability of less than about 1% (e.g., less than about 0.75%, less than about 0.50%, or less than about 0.30%).

As used herein, “DMARD” refers to a disease-modifying antirheumatoid drug. Examples of DMARDs include adalimumab, leflunomide, sulfasalazine, infliximab, minocycline, rituximab, golimumab, or any combination thereof.

### II. METHODS

One aspect of the present invention provides a method for treating or lessening the severity of a disease selected from spondyloarthropathy (e.g., peripheral spondyloarthropathy, axial spondyloarthropathy, reactive arthritis, Reiter's syndrome, psoriatic arthritis, ankylosing spondylitis, ulcerative colitis, Crohn's disease, or any combination thereof), rheumatoid arthritis (RA), systemic lupus erythematosus, or any combination thereof comprising administering to a patient in need thereof a chemotherapy agent and a compound of Formula I

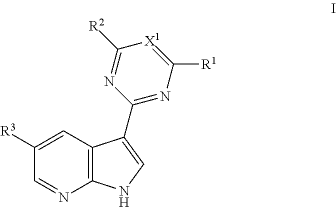

or a pharmaceutically acceptable salt thereof, wherein:

X¹ is N or CR⁴;

R² is H or halo;

R³ is H or halo;

R⁴ is H or halo;

R¹ is

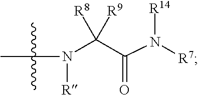

R″ is H or an unsubstituted C₁₋₂ aliphatic;

R⁸ is an unsubstituted C₁₋₄ aliphatic;

R⁹ is an unsubstituted C₁₋₄ aliphatic;

R⁷ is a C₁₋₃ aliphatic optionally substituted with up to 3 occurrences of F; and

R¹⁴ is H or unsubstituted C₁₋₂ alkyl.

In some embodiments, the chemotherapy agent comprises methotrexate, azathioprine (e.g., Imuran), cyclosporine, cyclophosphamide (e.g., Cytoxan), 6-mercaptopurine, or any combination thereof. And, in some instances, the chemotherapy agent comprises an injectable formulation or an oral formulation. For example, the chemotherapy agent comprises an injectable formulation or an oral formulation of methotrexate.

In other embodiments, the patient is administered from about 5 mg to about 100 mg of the chemotherapy agent (e.g., methotrexate) per month. The chemotherapy agent may be administered once per month or more than once per month (e.g., twice per month, three times per month, or four times per month).

In some embodiments, for the compound of Formula I, R² is H or F.

In some embodiments, R³ is H or Cl.

In some embodiments, each of R⁸ and R⁹ is independently selected from methyl, ethyl, propyl, iso-propyl, butyl, or tert-butyl. For instance, each of R⁸ and R⁹ is independently selected from methyl or ethyl.

In some embodiments, R¹⁴ is H or methyl.

In some embodiments, R⁷ is an unsubstituted C₁₋₃ aliphatic.

In some embodiments, R⁷ is a C₁₋₃ aliphatic substituted with 1-3 occurrences of F.

In some embodiments, R⁷ is a group selected from —CH₂CH₃, —CH₂CF₃, —CH₂CH₂CH₃, or —CHCH₃CH₃.

In some embodiments, the compound of Formula I is selected from Table 1:

TABLE 1

| | |
|---|---|
| 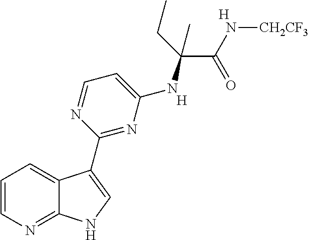 | 1 |
| 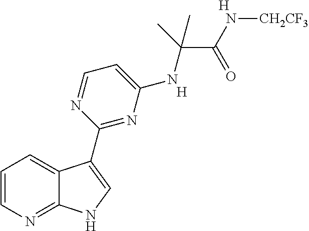 | 2 |
| 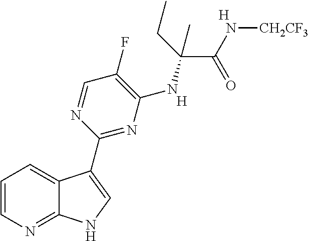 | 3 |
| 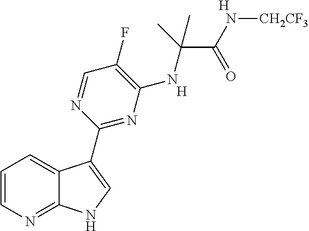 | 4 |
| 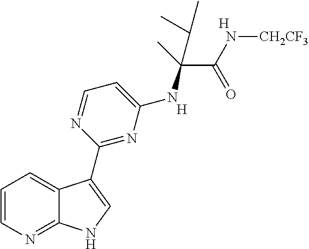 | 5 |
| 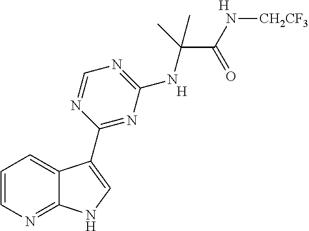 | 6 |
| 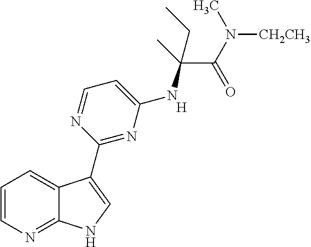 | 7 |
| 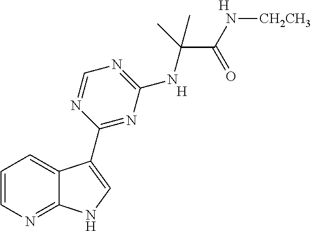 | 8 |
| 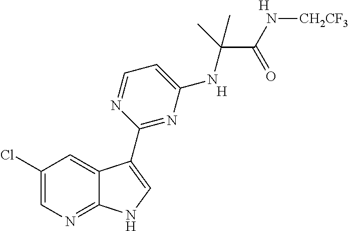 | 9 |
| 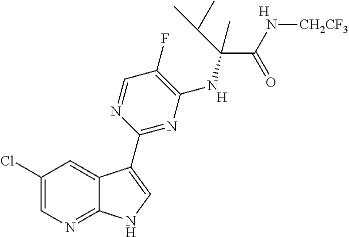 | 10 |
| 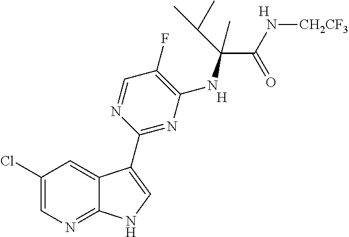 | 11 |
| 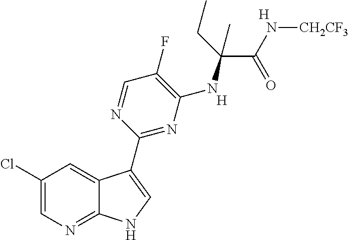 | 12 |
| 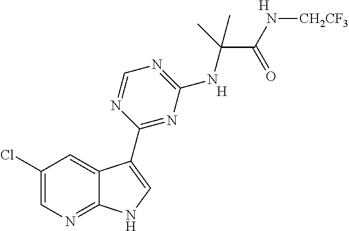 | 13 |
| 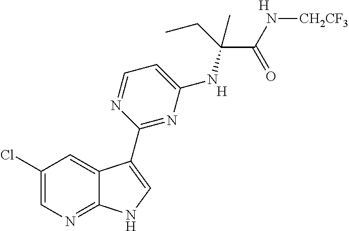 | 14 |
| 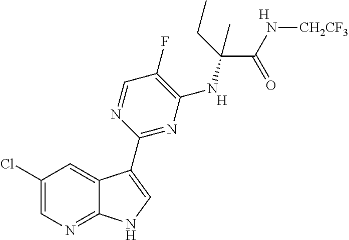 | 15 |
| 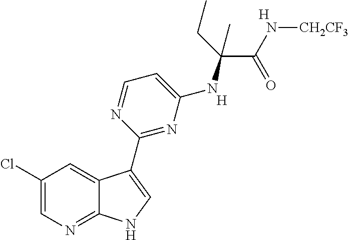 | 16 |
| 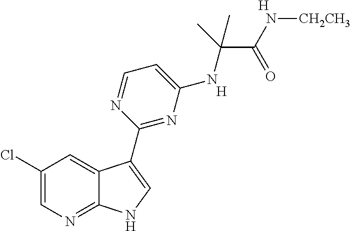 | 17 |
| 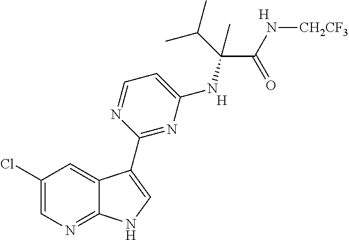 | 18 |
| 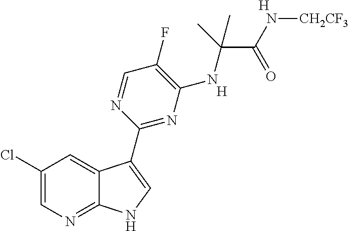 | 19 |

In some embodiments, the compound of Formula I is administered at least once per day (e.g., q.d. or b.i.d. administration). In other embodiments, the compound of Formula I is administered at least twice per day (e.g., b.i.d. administration).

In some embodiments, the compound of Formula I is orally administered to the patient.

In some embodiments, at least about 20 mg (e.g., at least about 25 mg, at least about 50 mg, at least about 75 mg, or at least about 100 mg) of the compound of Formula I is administered to the patient once per day. For example, at least about 150 mg of the compound of Formula I is administered to the patient once per day. In other examples, at least about 200 mg of the compound of Formula I is administered to the patient once per day.

In other embodiments, at least about 20 mg (e.g., at least about 25 mg, at least about 50 mg, at least about 75 mg, or at least about 100 mg) of the compound of Formula I is administered to the patient twice per day.

Another aspect of the present invention provides a method for treating or lessening the severity of a disease selected from systemic lupus erythematosus, peripheral spondyloarthropathy, axial spondyloarthropathy, ulcerative colitis, Crohn's disease, ankylosing spondylitis, reactive arthritis, Reiter's syndrome, psoriatic arthritis, or any combination thereof comprising administering to a patient in need thereof a compound of Formula I

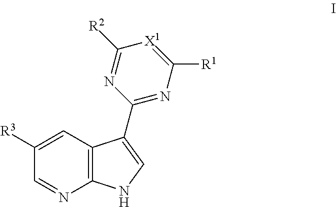

or a pharmaceutically acceptable salt thereof, wherein:

X¹ is N or CR⁴;

R² is H or halo;

R³ is H or halo;

R⁴ is H or halo;

R¹ is

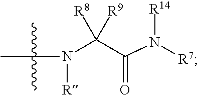

R″ is H or an unsubstituted C₁₋₂ aliphatic;

R⁸ is an unsubstituted C₁₋₄ aliphatic;

R⁹ is an unsubstituted C₁₋₄ aliphatic;

R⁷ is a C₁₋₃ aliphatic optionally substituted with up to 3 occurrences of F; and

R¹⁴ is H or unsubstituted C₁₋₂ alkyl.

Some embodiments further comprise administering one or more DMARDs (e.g., adalimumab, leflunomide, sulfasalazine, infliximab, minocycline, rituximab, golimumab, or any combination thereof) to the patient.

Some embodiments further comprise administering a chemotherapy agent to the patient. And, in some examples, the chemotherapy agent comprises methotrexate, azathioprine (e.g., Imuran), cyclosporine, cyclophosphamide (e.g., Cytoxan), 6-mercaptopurine, or any combination thereof.

In some embodiments, the chemotherapy agent (e.g., methotrexate) comprises an injectable formulation or an oral formulation.

In some embodiments, the patient is administered from about 5 mg to about 100 mg of the chemotherapy agent per month.

In some embodiments, for the compound of Formula I, R² is H or F.

In some embodiments, R³ is H or Cl.

In some embodiments, each of R⁸ and R⁹ is independently selected from methyl, ethyl, propyl, iso-propyl, butyl, or tert-butyl. For example, each of R⁸ and R⁹ is independently selected from methyl or ethyl.

In some embodiments, R¹⁴ is H or methyl.

In some embodiments, R⁷ is an unsubstituted C₁₋₃ aliphatic.

In some embodiments, R⁷ is a C₁₋₃ aliphatic substituted with 1-3 occurrences of F.

In some embodiments, R⁷ is a group selected from —CH₂CH₃, —CH₂CF₃, —CH₂CH₂CH₃, or —CHCH₃CH₃.

In some embodiments, the compound of Formula I is selected from the compounds in Table 1.

In some embodiments, the compound of Formula I is administered at least once per day. For example, the compound of Formula I is administered at least twice per day.

In some embodiments, the compound of Formula I is orally administered to the patient in need thereof.

In some embodiments, at least about 20 mg (e.g., at least about 50 mg, at least about 75 mg, or at least about 100 mg) of the compound of Formula I is administered to the patient once per day. For example, at least about 150 mg of the compound of Formula I is administered to the patient once per day. In other examples, at least about 200 mg of the compound of Formula I is administered to the patient once per day.

In other embodiments, at least about 20 mg (e.g., at least about 50 mg, at least about 75 mg, or at least about 100 mg) of the compound of Formula I is administered to the patient twice per day.

Another aspect of the present invention provides a method for treating or lessening the severity of a disease selected from rheumatoid arthritis, systemic lupus erythematosus, peripheral spondyloarthropathy, axial spondyloarthropathy, ulcerative colitis, Crohn's disease, ankylosing spondylitis, reactive arthritis, Reiter's syndrome, psoriatic arthritis, or any combination thereof comprising administering to a patient in need thereof a chemotherapy agent and a pharmaceutical composition comprising a compound of Formula I

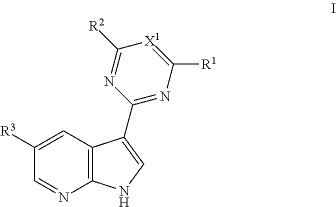

or a pharmaceutically acceptable salt thereof, wherein:

X¹ is N or CR⁴;

R² is H or halo;

R³ is H or halo;

R⁴ is H or halo;

R¹ is

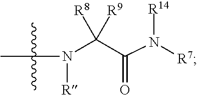

R″ is H or an unsubstituted C₁₋₂ aliphatic;

R⁸ is an unsubstituted C₁₋₄ aliphatic;

R⁹ is an unsubstituted C₁₋₄ aliphatic;

R⁷ is a C₁₋₃ aliphatic optionally substituted with up to 3 occurrences of F; and

R¹⁴ is H or unsubstituted C₁₋₂ alkyl.

In some embodiments, the chemotherapy agent comprises methotrexate, azathioprine (e.g., Imuran), cyclosporine, cyclophosphamide (e.g., Cytoxan), 6-mercaptopurine, or any combination thereof.

In some embodiments, the chemotherapy agent (e.g., methotrexate) comprises an injectable formulation or an oral formulation.

In some embodiments, the patient is administered from about 5 mg to about 100 mg of the chemotherapy agent per month.

In some embodiments, for the compound of Formula I, R² is H or F.

In some embodiments, R³ is H or Cl.

In some embodiments, each of R⁸ and R⁹ is independently selected from methyl, ethyl, propyl, iso-propyl, butyl, or tert-butyl. For example, each of R⁸ and R⁹ is independently selected from methyl or ethyl.

In some embodiments, R¹⁴ is H or methyl.

In some embodiments, R⁷ is an unsubstituted C₁₋₃ aliphatic.

In some embodiments, R⁷ is a C₁₋₃ aliphatic substituted with 1-3 occurrences of F.

In some embodiments, R⁷ is a group selected from —CH₂CH₃, —CH₂CF₃, —CH₂CH₂CH₃, or —CHCH₃CH₃.

In some embodiments, the compound of Formula I is selected from the compounds in Table 1.

In some embodiments, the pharmaceutical composition further comprises a tablet. And, in some of these embodiments, the tablet further comprises a diluent, a binder, a glidant, a disintegrant, a surfactant, a lubricant, or any combination thereof.

In some embodiments, the tablet is administered at least once per day (e.g., q.d. or b.i.d. administration).

In some embodiments, the tablet comprises at least about 10 mg (e.g., at least about 15 mg, at least about 20 mg, at least about 25 mg) of the compound of Formula I.

In some embodiments, the tablet comprises from about 5 mg to about 150 mg (e.g., from about 10 mg to about 100 mg, from about 20 mg to about 75 mg, or from about 25 mg to about 50 mg) of the compound of Formula I.

In some embodiments, the tablet is administered at least twice per day (e.g., b.i.d. administration).

Some embodiments further comprise administering once per day at least one tablet comprising the pharmaceutical composition. For example, some embodiments further comprise administering once per day at least one table comprising from about 5 mg to about 150 mg (e.g., from about 10 mg to about 100 mg, from about 20 mg to about 75 mg, or from about 25 mg to about 50 mg) of the compound of Formula I.

Some embodiments further comprise administering twice per day at least one tablet comprising the pharmaceutical composition. For example, some embodiments further comprise administering twice per day at least one table comprising from about 5 mg to about 150 mg (e.g., from about 10 mg to about 100 mg, from about 20 mg to about 75 mg, or from about 25 mg to about 50 mg) of the compound of Formula I.

In several embodiments, the pharmaceutical composition comprises:

a. a compound of Formula I

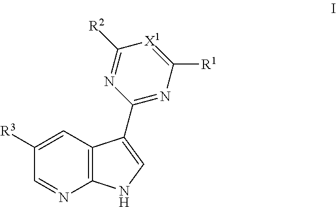

or a pharmaceutically acceptable salt thereof, wherein:

X¹ is N or CR⁴;

R² is H or halo;

R⁴ is H or halo;

R¹ is

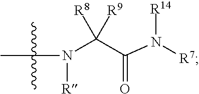

R″ is H or an unsubstituted C₁₋₂ aliphatic;

R⁸ is an unsubstituted C₁₋₄ aliphatic;

R⁹ is an unsubstituted C₁₋₄ aliphatic

R⁷ is a C₁₋₃ aliphatic optionally substituted with up to 3 occurrences of F; and

R¹⁴ is H or unsubstituted C₁₋₂ alkyl;

b. a diluent;

c. a disintegrant;

d. a wetting agent;

e. a binder;

f. a glidant; and

g. a lubricant.

In some embodiments, X¹ is N, CH, or CF.

In some embodiments, R″ is H or methyl.

In some embodiments, R² is H or F.

In some embodiments, R⁸ is an unsubstituted C₁₋₄ aliphatic, for example a straight or branched unsubstituted C₁₋₄ aliphatic.

In some embodiments, R⁹ is an unsubstituted C₁₋₄ aliphatic, for example a straight or branched unsubstituted C₁₋₄ aliphatic.

In some embodiments, each of R⁸ and R⁹ is independently selected from methyl, ethyl, propyl, iso-propyl, butyl, or tert-butyl, each of which is unsubstituted. For example, each of R⁸ and R⁹ is independently selected from methyl or ethyl.

In some embodiments, R¹⁴ is H or methyl.

In some embodiments, R⁷ is an unsubstituted C₁₋₃ aliphatic. For example, R⁷ is a straight or branched unsubstituted C₁₋₃ aliphatic.

In some embodiments, R⁷ is a C₁₋₃ aliphatic substituted with 1-3 occurrences of F.

In some embodiments, R⁷ is a group selected from —CH₂CH₃, —CH₂CF₃, —CH₂CH₂CH₃, or —CHCH₃CH₃.

In some embodiments, the pharmaceutical composition comprises from about 20 mg to about 250 mg (e.g., from about 25 mg to about 200 mg, from about 50 mg to about 175 mg, or from about 75 mg to about 150 mg) of a compound of Formula I.

In some embodiments, the pharmaceutical composition comprises about 25 mg of a compound of Formula I.

In some embodiments, the pharmaceutical composition comprises about 50 mg of a compound of Formula I.

In some embodiments, the pharmaceutical composition comprises about 75 mg of a compound of Formula I.

In some embodiments, the pharmaceutical composition comprises about 100 mg of a compound of Formula I.

In some embodiments, the pharmaceutical composition comprises about 150 mg of a compound of Formula I.

In some embodiments, the pharmaceutical composition comprises about 25 mg of a compound selected from Table 1.

In some embodiments, the pharmaceutical composition comprises about 50 mg of a compound selected from Table 1.

In some embodiments, the pharmaceutical composition comprises about 75 mg of a compound selected from Table 1.

In some embodiments, the pharmaceutical composition comprises about 100 mg of a compound selected from Table 1.

In some embodiments, the pharmaceutical composition comprises about 150 mg of a compound selected from Table 1.

Another aspect of the present invention provides a method for treating or lessening the severity of a disease selected from spondyloarthropathy (e.g., peripheral spondyloarthropathy, axial spondyloarthropathy, reactive arthritis, Reiter's syndrome, psoriatic arthritis, ankylosing spondylitis, ulcerative colitis, Crohn's disease, or any combination thereof), systemic lupus erythematosus, rheumatoid arthritis (RA), or any combination thereof comprising administering to a patient in need thereof a pharmaceutical composition comprising a compound of Formula I

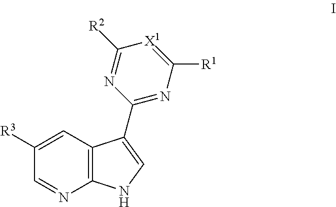

or a pharmaceutically acceptable salt thereof, wherein:

X¹ is N or CR⁴;

R² is H or halo;

R³ is H or halo;

R⁴ is H or halo;

R¹ is

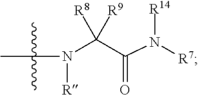

R″ is H or an unsubstituted C₁₋₂ aliphatic;

R⁸ is an unsubstituted C₁₋₄ aliphatic;

R⁹ is an unsubstituted C₁₋₄ aliphatic;

R⁷ is a C₁₋₃ aliphatic optionally substituted with up to 3 occurrences of F; and

R¹⁴ is H or unsubstituted C₁₋₂ alkyl.

In some embodiments, R² is H or F.

In some embodiments, R³ is H or Cl.

In some embodiments, each of R⁸ and R⁹ is independently selected from methyl, ethyl, propyl, iso-propyl, butyl, or tert-butyl. For example, each of R⁸ and R⁹ is independently selected from methyl or ethyl.

In some embodiments, R¹⁴ is H or methyl.

In some embodiments, R⁷ is an unsubstituted C₁₋₃ aliphatic.

In some embodiments, R⁷ is a C₁₋₃ aliphatic substituted with 1-3 occurrences of F.

In some embodiments, R⁷ is a group selected from —CH₂CH₃, —CH₂CF₃, —CH₂CH₂CH₃, or —CHCH₃CH₃.

In some embodiments, the compound of Formula I is selected from the compounds in Table 1.

In some embodiments, the pharmaceutical composition further comprises a tablet. And, in some of these embodiments, the tablet further comprises a diluent, a binder, a glidant, a disintegrant, a surfactant, a lubricant, or any combination thereof.

In some embodiments, the tablet is administered at least once per day (e.g., q.d. or b.i.d. administration).

In some embodiments, the tablet comprises at least about 10 mg (e.g., at least about 15 mg, at least about 20 mg, at least about 25 mg) of the compound of Formula I.

In some embodiments, the tablet comprises from about 5 mg to about 150 mg (e.g., from about 10 mg to about 100 mg, from about 20 mg to about 75 mg, or from about 25 mg to about 50 mg) of the compound of Formula I.

In some embodiments, the tablet is administered at least twice per day (e.g., b.i.d. administration).

Some embodiments further comprise administering once per day at least one tablet comprising the pharmaceutical composition. For example, some embodiments further comprise administering once per day at least one table comprising from about 5 mg to about 150 mg (e.g., from about 10 mg to about 100 mg, from about 20 mg to about 75 mg, or from about 25 mg to about 50 mg) of the compound of Formula I.

Some embodiments further comprise administering twice per day at least one tablet comprising the pharmaceutical composition. For example, some embodiments further comprise administering twice per day at least one table comprising from about 5 mg to about 150 mg (e.g., from about 10 mg to about 100 mg, from about 20 mg to about 75 mg, or from about 25 mg to about 50 mg) of the compound of Formula I.

In several embodiments, the pharmaceutical composition comprises:

a. a compound of Formula I

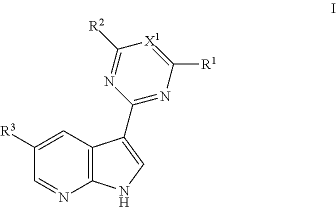

or a pharmaceutically acceptable salt thereof, wherein:

X¹ is N or CR⁴;

R² is H or halo;

R⁴ is H or halo;

R¹ is

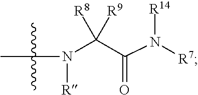

R″ is H or an unsubstituted C₁₋₂ aliphatic;

R⁸ is an unsubstituted C₁₋₄ aliphatic;

R⁹ is an unsubstituted C₁₋₄ aliphatic

R⁷ is a C₁₋₃ aliphatic optionally substituted with up to 3 occurrences of F; and

R¹⁴ is H or unsubstituted C₁₋₂ alkyl;

b. a diluent;

c. a disintegrant;

d. a wetting agent;

e. a binder;

f. a glidant; and

g. a lubricant.

In some embodiments, X¹ is N, CH, or CF.

In some embodiments, R″ is H or methyl.

In some embodiments, R² is H or F.

In some embodiments, R⁸ is an unsubstituted C₁₋₄ aliphatic, for example a straight or branched unsubstituted C₁₋₄ aliphatic.

In some embodiments, R⁹ is an unsubstituted C₁₋₄ aliphatic, for example a straight or branched unsubstituted C₁₋₄ aliphatic.

In some embodiments, each of R⁸ and R⁹ is independently selected from methyl, ethyl, propyl, iso-propyl, butyl, or tert-butyl, each of which is unsubstituted. For example, each of R⁸ and R⁹ is independently selected from methyl or ethyl.

In some embodiments, R¹⁴ is H or methyl.

In some embodiments, R⁷ is an unsubstituted C₁₋₃ aliphatic. For example, R⁷ is a straight or branched unsubstituted C₁₋₃ aliphatic.

In some embodiments, R⁷ is a C₁₋₃ aliphatic substituted with 1-3 occurrences of F.

In some embodiments, R⁷ is a group selected from —CH₂CH₃, —CH₂CF₃, —CH₂CH₂CH₃, or —CHCH₃CH₃.

In some embodiments, the pharmaceutical composition comprises about 25 mg of a compound of Formula I. In some embodiments, the pharmaceutical composition comprises about 50 mg of a compound of Formula I. In some embodiments, the pharmaceutical composition comprises about 75 mg of a compound of Formula I. In some embodiments, the pharmaceutical composition comprises about 100 mg of a compound of Formula I. In some embodiments, the pharmaceutical composition comprises about 150 mg of a compound of Formula I.

In some embodiments, the pharmaceutical composition comprises about 25 mg of a compound selected from Table 1. In some embodiments, the pharmaceutical composition comprises about 50 mg of a compound selected from Table 1. In some embodiments, the pharmaceutical composition comprises about 75 mg of a compound selected from Table 1. In some embodiments, the pharmaceutical composition comprises about 100 mg of a compound selected from Table 1. In some embodiments, the pharmaceutical composition comprises about 150 mg of a compound selected from Table 1.

Another aspect of the present invention provides a method of treating or reducing the severity of a disease selected from rheumatoid arthritis, systemic lupus erythematosus, peripheral spondyloarthropathy, axial spondyloarthropathy, ulcerative colitis, Crohn's disease, ankylosing spondylitis, reactive arthritis, Reiter's syndrome, psoriatic arthritis, or any combination thereof comprising administering to a patient once daily or twice daily a pharmaceutical composition comprising a compound of Formula I and administering a chemotherapy agent (e.g., methotrexate), wherein the pharmaceutical composition is as described herein.

In some embodiments, the pharmaceutical composition comprising a JAK inhibitor API (e.g., a compound of Formula I) and optionally other excipients (e.g., a diluent, a disintegrant, a wetting agent, a binder, a glidant, a colorant, a lubricant, or any combination thereof).

In some embodiments, the pharmaceutical composition comprises:
a. a compound of Formula I, as described above;
b. a diluent;
c. a disintegrant;
d. a surfactant;
e. a binder;
f. a glidant; and
g. a lubricant.

In other embodiments, the pharmaceutical composition comprises about 25 mg of a compound of Formula I, a diluent, a disintegrant, a surfactant, a binder, a glidant, and a lubricant.

In other embodiments, the pharmaceutical composition comprises about 50 mg of a compound of Formula I, a diluent, a disintegrant, a surfactant, a binder, a glidant, and a lubricant.

In other embodiments, the pharmaceutical compositions of the present invention also comprise one or more excipients such as diluents, disintegrants, surfactants, binders, glidants, lubricants, colorants, or fragrances, such as any of those described below.

In some embodiments, the pharmaceutical composition can comprise tablets and the tablets can be coated with a colorant and optionally labeled with a logo, other image and/or text using a suitable ink. In still other embodiments, the pharmaceutical composition can be made into tablets and the tablets can be coated with a colorant, waxed, and optionally labeled with a logo, other image and/or text using a suitable ink. Suitable colorants and inks are compatible with the ingredients of the pharmaceutical composition, i.e., they do not substantially reduce the solubility, the chemical stability, the physical stability, the hardness, or the biological activity of the pharmaceutical composition. The suitable colorants and inks can be any color and are water based or solvent based. In some embodiments, tablets made from the pharmaceutical composition are coated with a colorant and then labeled with a logo, other image, and/or text using a suitable ink. For example, tablets comprising a pharmaceutical composition as described herein can be coated with about 3 wt % (e.g., less than about 6 wt % or less than about 4 wt %) of film coating comprising a colorant. The colored tablets can be labeled with a logo and text indicating the strength of the active ingredient in the tablet using a suitable ink. The colored tablets can be labeled with a logo and text indicating the strength and/or mass of the active ingredient in the tablet using a black ink (e.g., Opacode® WB, commercially available from Colorcon, Inc. of West Point, Pa.). In another embodiment, tablets made from the pharmaceutical composition are coated with a colorant, waxed, and then labeled with a logo, other image, and/or text using a suitable ink. The colored tablets can be waxed with Carnauba wax powder weighed out in the amount of about 0.01% w/w of the starting tablet core weight. The waxed tablets can be labeled with a logo and text indicating the strength of the active ingredient in the tablet using a suitable ink.

In some embodiments, the pharmaceutical composition comprises from about 5 wt % to about 50 wt % of a compound of Formula I, by weight of the composition; from about 25 wt % to about 50 wt % of a diluent; from about 1 wt % to about 10 wt % of a disintegrant; from about 2 wt % to about 0.3 wt % of a wetting agent (e.g., surfactant); from about 5 wt % to about 50 wt % of a binder; from about 2 wt % to about 0.05 wt % of a glidant; and from about 2 wt % to about 0.1 wt % of a lubricant. Or, the pharmaceutical composition comprises from about 35 wt % to about 50 wt % of a compound of Formula I; from about 25 wt % to about 50 wt % of a diluent; from about 1 wt % to about 10 wt % of a disintegrant; from about 2 wt % to about 0.3 wt % of a wetting agent (e.g., surfactant); from about 5 wt % to about 50 wt % of a binder; from about 2 wt % to about 0.05 wt % of a glidant; and from about 2 wt % to about 0.1 wt % of a lubricant.

In some embodiments, the pharmaceutical composition comprises from about 30 wt % to about 50 wt % of a compound of Formula I; from about 35 wt % to about 55 wt % of microcrystalline cellulose by weight of the composition; from about 35 wt % to about 55 wt % of lactose by weight of the composition; from about 1 wt % to about 5 wt % of sodium croscarmellose by weight of the composition; from about 0.5 wt % to about 1.5 wt % of SLS by weight of the composition; from about 0.5 wt % to about 1.5 wt % of colloidal silicon dioxide by weight of the composition; and from about 0.5 wt % to about 1.0 wt % of magnesium stearate by weight of the composition.

Or, the pharmaceutical composition of the present invention comprises about 20 wt % of a compound of Formula I, about 37 wt % of microcrystalline cellulose by weight of the composition, about 37 wt % of lactose by weight of the composition, about 3 wt % of sodium croscarmellose by weight of the composition, about 1 wt % of SLS by weight of the composition, about 1 wt % of colloidal silicon dioxide by weight of the composition, and about 0.75 wt % of magnesium stearate by weight of the composition.

In some embodiments, the pharmaceutical composition of the present invention comprises about 10 wt % of a compound of Formula I; about 42 wt % of microcrystalline cellulose by weight of the composition; about 42 wt % of lactose by weight of the composition; about 3 wt % of sodium croscarmellose by weight of the composition; about 1 wt % of SLS by weight of the composition; about 1 wt % of colloidal silicon dioxide by weight of the composition; and about 0.75 wt % of magnesium stearate by weight of the composition.

In some embodiments, the pharmaceutical composition consists of a tablet that comprises a protein kinase inhibitor API (e.g., a compound of Formula I) and other excipients (e.g., a filler, a disintegrant, a surfactant, a binder, a glidant, a colorant, a lubricant, or any combination thereof), each of which is described above and in the Examples below, wherein the tablet has a hardness of about 5 Kp or greater. In one example, the pharmaceutical composition consists of a tablet that comprises a JAK inhibitor API (e.g., a compound of Formula I) and other excipients (e.g., a filler, a disintegrant, a surfactant, a binder, a glidant, a colorant, a lubricant, or any combination thereof), each of which is described above and in the Examples below, wherein the tablet has a hardness of about 5 Kp or greater (e.g., about 5.5 Kp or greater, about 6 Kp or greater, or about 7 Kp or greater).

In some embodiments, the pharmaceutical composition comprises a compound of Formula I; a diluent; a disintegrant; a wetting agent; a binder; a glidant; and a lubricant.

In some embodiments, the diluent is lactose, sorbitol, cellulose, calcium phosphate, starch, sugar, or any combination thereof.

In some embodiments, the diluent is lactose and has a concentration of about 10 wt % or greater by weight of the composition.

In some embodiments, the disintegrant is sodium croscarmellose, sodium starch glycolate, or a combination thereof. For example the disintegrant is sodium croscarmellose and has a concentration of about 10 wt % or less by weight of the composition.

In some embodiments, the wetting agent is sodium lauryl sulfate, sodium stearyl fumarate, polyoxyethylene 20 sorbitan mono-oleate, or any combination thereof. For example, the wetting agent is sodium lauryl sulfate and has a concentration of about 10 wt % or less by weight of the composition.

In some embodiments, the binder is microcrystalline cellulose, dibasic calcium phosphate, sucrose, corn starch, modified cellulose, or any combination thereof. For example, the binder is microcrystalline cellulose and has a concentration of about 1 wt % or greater by weight of the composition.

In some embodiments, the glidant is colloidal silicon dioxide, talc, or a combination thereof. For example, the glidant is colloidal silicon dioxide and has a concentration of 2 wt % or less by weight of the composition.

In some embodiments, the lubricant is magnesium stearate, stearic acid, hydrogenated oil, sodium stearyl fumarate, or any combination thereof. For example, the lubricant is magnesium stearate and has a concentration of less than about 2 wt % by weight of the composition.

In some embodiments, the pharmaceutical composition further comprises a colorant.

Another aspect of the present invention provides a method for treating or lessening the severity of a disease selected from peripheral spondyloarthropathy, axial spondyloarthropathy, reactive arthritis, Reiter's syndrome, psoriatic arthritis, ankylosing spondylitis, ulcerative colitis, Crohn's disease, systemic lupus erythematosus, or any combination thereof comprising administering to a patient in need thereof a compound of Formula I

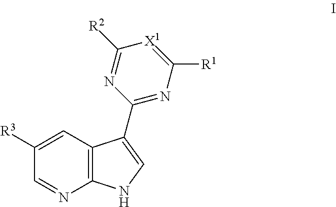

or a pharmaceutically acceptable salt thereof, wherein:

X¹ is N or CR⁴;

R² is H or halo;

R⁴ is H or halo;

R¹ is

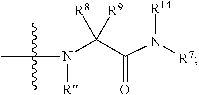

R″ is H or an unsubstituted C₁₋₂ aliphatic;

R⁸ is an unsubstituted C₁₋₄ aliphatic;

R⁹ is an unsubstituted C₁₋₄ aliphatic

R⁷ is a C₁₋₃ aliphatic optionally substituted with up to 3 occurrences of F; and

R¹⁴ is H or unsubstituted C₁₋₂ alkyl.

Some embodiments further comprise administering to the patient a chemotherapy agent. In some examples, the chemotherapy agent is selected from methotrexate, azathioprine (e.g., Imuran), cyclosporine, cyclophosphamide (e.g., Cytoxan), 6-mercaptopurine, or any combination thereof.

In some embodiments, the compound of Formula I is administered to the patient once per day. For example, about 100 mg of the compound of Formula I is administered to the patient once per day. In other examples, about 150 mg of the compound of Formula I is administered to the patient once per day. And, in some examples, about 200 mg of the compound of Formula I is administered to the patient once per day.

In other embodiments, the compound of Formula I is administered twice per day. For example, about 100 mg of the compound of Formula I is administered to the patient twice per day.

In some embodiments, R² is H or F.

In some embodiments, each of R⁸ and R⁹ is independently selected from methyl, ethyl, propyl, iso-propyl, butyl, or tert-butyl. For example, each of R⁸ and R⁹ is independently selected from methyl or ethyl.

In some embodiments, R¹⁴ is H or methyl.

In some embodiments, R⁷ is an unsubstituted C₁₋₃ aliphatic.

In some embodiments, R⁷ is a C₁₋₃ aliphatic substituted with 1-3 occurrences of F.

In some embodiments, R⁷ is a group selected from —CH₂CH₃, —CH₂CF₃, —CH₂CH₂CH₃, or —CHCH₃CH₃.

In some embodiments, the compound of Formula I is one selected from Table 1.

Another aspect of the present invention provides a method for treating or lessening the severity of a disease selected from spondyloarthropathy (e.g., peripheral spondyloarthropathy, axial spondyloarthropathy, reactive arthritis, Reiter's syndrome, psoriatic arthritis, ankylosing spondylitis, ulcerative colitis, Crohn's disease, or any combination thereof), systemic lupus erythematosus, rheumatoid arthritis (RA), or any combination thereof comprising administering to a patient in need thereof a compound of Formula I

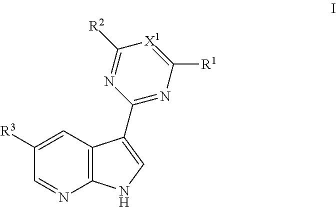

or a pharmaceutically acceptable salt thereof, wherein:

X¹ is N or CR⁴;

R² is H or halo;

R⁴ is H or halo;

R¹ is

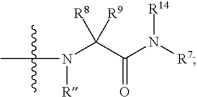

R″ is H or an unsubstituted C₁₋₂ aliphatic;

R⁸ is an unsubstituted C₁₋₄ aliphatic;

R⁹ is an unsubstituted C₁₋₄ aliphatic

R⁷ is a C₁₋₃ aliphatic optionally substituted with up to 3 occurrences of F; and

R¹⁴ is H or unsubstituted C₁₋₂ alkyl,
wherein at least about 100 mg of the compound of Formula I is administered to the patient at least once per day.

In some embodiments, about 100 mg of the compound of formula I is administered to the patient once per day.

In other embodiments, about 150 mg of the compound of formula I is administered to the patient once per day.

In other embodiments, about 200 mg of the compound of formula I is administered to the patient once per day.

And, in some embodiments, about 100 mg of the compound of formula I is administered to the patient twice per day.

Some embodiments further comprise administering to the patient a chemotherapy agent. And, in some embodiments, the chemotherapy agent is selected from methotrexate, azathioprine (e.g., Imuran), cyclosporine, cyclophosphamide (e.g., Cytoxan), 6-mercaptopurine, or any combination thereof.

In some embodiments, R² is H or F.

In some embodiments, each of R⁸ and R⁹ is independently selected from methyl, ethyl, propyl, iso-propyl, butyl, or tert-butyl. For example, each of R⁸ and R⁹ is independently selected from methyl or ethyl.

In some embodiments, R¹⁴ is H or methyl.

In some embodiments, R⁷ is an unsubstituted C₁₋₃ aliphatic.

In some embodiments, R⁷ is a C₁₋₃ aliphatic substituted with 1-3 occurrences of F.

In some embodiments, R⁷ is a group selected from —CH₂CH₃, —CH₂CF₃, —CH₂CH₂CH₃, or —CHCH₃CH₃.

In some embodiments, the compound of Formula I is one selected from Table 1.

In some embodiments, the compound of Formula I is administered to the patient in the form of an oral tablet. In some examples, the tablet comprises 50 mg of the compound of Formula I (e.g., Tablet 1, described below). In embodiments wherein 100 mg of the compound of Formula I is administered, either once per day (q.d.) or twice per day (b.i.d.), the administration may further include the oral administration of two 50 mg tablets (e.g., 2× Tablet 1) once per day or twice per day depending on the dosage regime. In embodiments wherein 150 mg of the compound of Formula I is administered, either once per day (q.d.) or twice per day (b.i.d.), the administration may further include the oral administration of three of the 50 mg tablets (e.g., 3× Tablet 1) once per day or twice per day depending on the dosage regime. And, in embodiments wherein 200 mg of the compound of Formula I is administered, either once per day (q.d.) or twice per day (b.i.d.), the administration may further include the oral administration of four 50 mg tablets (e.g., 4× Tablet 1) once per day or twice per day depending on the dosage regime.

### III. PHARMACEUTICAL COMPOSITIONS

The pharmaceutical compositions of the present invention are useful in the methods for treating or lessening the severity of a disease selected from spondyloarthropathy (e.g., peripheral spondyloarthropathy, axial spondyloarthropathy, reactive arthritis, Reiter's syndrome, psoriatic arthritis, ankylosing spondylitis, ulcerative colitis, Crohn's disease, or any combination thereof), rheumatoid arthritis (RA), systemic lupus erythematosus, or any combination thereof.

One aspect of the present invention provides a pharmaceutical composition comprising a compound of Formula I

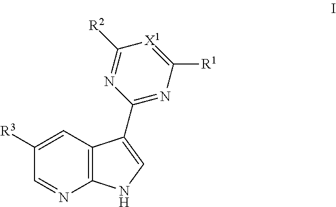

or a pharmaceutically acceptable salt thereof, wherein:

X¹ is N or CR⁴;

R² is H or halo;

R⁴ is H or halo;

R¹ is

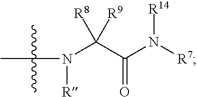

R″ is H or an unsubstituted C₁₋₂ aliphatic;

R⁸ is an unsubstituted C₁₋₄ aliphatic;

R⁹ is an unsubstituted C₁₋₄ aliphatic;

R⁷ is a C₁₋₃ aliphatic optionally substituted with up to 3 occurrences of F; and

R¹⁴ is H or unsubstituted C₁₋₂ alkyl; and

one or more excipients comprising a diluent, a disintegrant, a wetting agent, a binder, a glidant, a lubricant, or any combination thereon, wherein the compound of Formula I has a concentration of from about 25 wt % to about 60 wt % by weight of the composition, and the total concentration for the one or more excipients is from about 40 wt % to about 75 wt % by weight of the composition.

In some embodiments, X¹ is N, CH, or CF.

In some embodiments, R″ is H or methyl.

In some embodiments, R² is H or F.

In some embodiments, R⁸ is an unsubstituted C₁₋₄ aliphatic, for example a straight or branched unsubstituted C₁₋₄ aliphatic.

In some embodiments, R⁹ is an unsubstituted C₁₋₄ aliphatic, for example a straight or branched unsubstituted C₁₋₄ aliphatic.

In some embodiments, each of R⁸ and R⁹ is independently selected from methyl, ethyl, propyl, iso-propyl, butyl, or tert-butyl. For example, each of R⁸ and R⁹ is independently selected from methyl or ethyl.

In some embodiments, R¹⁴ is H or methyl.

In some embodiments, R⁷ is an unsubstituted C₁₋₃ aliphatic. For example, R⁷ is a straight or branched unsubstituted C₁₋₃ aliphatic.

In some embodiments, R⁷ is a C₁₋₃ aliphatic substituted with 1-3 occurrences of F.

In some embodiments, R⁷ is a group selected from —CH₂CH₃, —CH₂CF₃, —CH₂CH₂CH₃, or —CHCH₃CH₃.

In some embodiments, the compound of Formula I is one selected from the compounds of Table 1.

In some embodiments, the pharmaceutical composition comprises at least about 10 mg (e.g., at least about 15 mg, at least about 20 mg, at least about 25 mg) of the compound of Formula I.

In some embodiments, the tablet comprises from about 5 mg to about 150 mg (e.g., from about 10 mg to about 100 mg, 20 mg to about 75 mg, or from about 25 mg to about 50 mg) of the compound of Formula I.

In some embodiments, the pharmaceutical composition comprises from about 10 mg to about 400 mg (e.g., from about 120 mg to about 380 mg, from about 130 mg to about 360 mg, or from about 150 mg to about 350 mg) of a compound of Formula I.

In some embodiments, the pharmaceutical composition comprises about 160 mg of a compound of Formula I.

In some embodiments, the pharmaceutical composition comprises about 175 mg of a compound of Formula I.

In some embodiments, the pharmaceutical composition comprises about 200 mg of a compound of Formula I.

In some embodiments, the pharmaceutical composition comprises about 250 mg of a compound of Formula I.

In some embodiments, the pharmaceutical composition comprises about 300 mg of a compound of Formula I.

In some embodiments, the pharmaceutical composition comprises from about 100 mg to about 400 mg (e.g., from about 120 mg to about 380 mg, from about 130 mg to about 360 mg, or from about 150 mg to about 350 mg) of a compound of Formula I.

In some embodiments, the pharmaceutical composition comprises about 160 mg of a compound of Formula I.

In some embodiments, the pharmaceutical composition comprises about 175 mg of a compound of Formula I.

In some embodiments, the pharmaceutical composition comprises about 200 mg of a compound of Formula I.

In some embodiments, the pharmaceutical composition comprises about 250 mg of a compound of Formula I.

In some embodiments, the pharmaceutical composition comprises about 300 mg of a compound of Formula I.

In some embodiments, the pharmaceutical composition comprises about 200 mg, about 225 mg, about 250 mg, about 275 mg, or about 300 mg of a compound selected from Table 1.

In some embodiments, the pharmaceutical composition comprises about 25 wt % to about 35 wt % of the compound of Formula I by weight of the composition. For example, the composition comprises 25 wt % or 30 wt % of the compound of Formula I.

In other embodiments, the pharmaceutical composition comprises from about 45 wt % to about 55 wt % of the compound of Formula I by weight of the composition.

And, in some embodiments, the pharmaceutical composition comprises a tablet or capsule. For example, the pharmaceutical composition comprises a tablet.

In other embodiments, the pharmaceutical compositions of the present invention also comprise one or more excipients such as diluents, disintegrants, surfactants, binders, glidants, lubricants, colorants, or fragrances.

Diluents suitable for the present invention are compatible with the ingredients of the pharmaceutical composition, i.e., they do not substantially reduce the solubility, the hardness, the chemical stability, the physical stability, or the biological activity of the pharmaceutical composition. Exemplary diluents include lactose, sorbitol, celluloses, calcium phosphates, starches, sugars (e.g., mannitol, sucrose, or the like), or any combination thereof. In one embodiment, the pharmaceutical composition comprises at least one diluent in an amount of about 10 wt % or greater (e.g., about 20 wt % or greater, about 25 wt % or greater, or about 35 wt % or greater) by weight of the composition. For example, the pharmaceutical composition comprises from about 30 wt % to about 50 wt % (e.g., from about 35 wt % to about 45 wt %), by weight of the composition, of at least one diluent. In another example, the pharmaceutical composition comprises from about 40 wt % to about 60 wt % (e.g., from about 45 wt % to about 55 wt %), by weight of the composition, of at least one diluent. In another example, the pharmaceutical composition comprises about 20 wt % or greater (e.g., about 25 wt % or greater, or about 30 wt % or greater) of lactose, by weight of the composition. In yet another example, the pharmaceutical composition comprises from about 20 wt % to about 60 wt % (e.g., from about 25 wt % to about 55 wt % or from about 27 wt % to about 45 wt %) of lactose, by weight of the composition.

Disintegrants suitable for the present invention enhance the dispersal of the pharmaceutical composition and are compatible with the ingredients of the pharmaceutical composition, i.e., they do not substantially reduce the chemical stability, the physical stability, the hardness, or the biological activity of the pharmaceutical composition. Exemplary disintegrants include sodium croscarmellose, sodium starch glycolate, or a combination thereof. In one embodiment, the pharmaceutical composition comprises disintegrant in an amount of about 10 wt % or less (e.g., about 9 wt % or less, about 8.5 wt % or less, about 8 wt % or less, or about 7.5 wt % or less) by weight of the composition. For example, the pharmaceutical composition comprises from about 1 wt % to about 10 wt % (e.g., from about 1 wt % to about 9 wt % or from about 2 wt % to about 8 wt %) of disintegrant, by weight of the composition. In another example, the pharmaceutical composition comprises about 10 wt % or less (e.g., about 9 wt % or less, about 8 wt % or less, or about 7.5 wt % or less) of sodium croscarmellose, by weight of the composition. In some examples, the pharmaceutical composition comprises from about 0.1% to about 10 wt % (e.g., from about 0.5 wt % to about 7.5 wt % or from about 1.5 wt % to about 6 wt %) of disintegrant, by weight of the composition. In still other examples, the pharmaceutical composition comprises from about 0.5% to about 10 wt % (e.g., from about 1.5 wt % to about 7.5 wt % or from about 2.5 wt % to about 6 wt %) of disintegrant, by weight of the composition.

Wetting agents (e.g., surfactants) suitable for the present invention enhance the solubility of the pharmaceutical composition and are compatible with the ingredients of the pharmaceutical composition, i.e., they do not substantially reduce the chemical stability, the physical stability, the hardness, or the biological activity of the pharmaceutical composition. Exemplary surfactants include sodium lauryl sulfate (SLS), sodium stearyl fumarate (SSF), polyoxyethylene 20 sorbitan mono-oleate (e.g., Tween™), any combination thereof, or the like. In one embodiment, the pharmaceutical composition comprises a surfactant in an amount of about 10 wt % or less (e.g., about 5 wt % or less, about 2 wt % or less, or about 1.5 wt % or less) by weight of the composition. For example, the pharmaceutical composition includes from about 10 wt % to about 0.1 wt % (e.g., from about 5 wt % to about 0.2 wt % or from about 2 wt % to about 0.3 wt %) of surfactant, by weight of the composition. In another example, the pharmaceutical composition comprises 10 wt % or less (e.g., about 5 wt % or less, about 2 wt % or less, about 1 wt % or less, about 0.8 wt % or less, or about 0.6 wt % or less) of sodium lauryl sulfate, by weight of the composition. In yet another example, the pharmaceutical composition comprises from about 10 wt % to about 0.1 wt % (e.g., from about 5 wt % to about 0.2 wt % or from about 2 wt % to about 0.3 wt %) of sodium lauryl sulfate, by weight of the composition.

Binders suitable for the present invention enhance the tablet strength of the pharmaceutical composition and are compatible with the ingredients of the pharmaceutical composition, i.e., they do not substantially reduce the chemical stability, the physical stability, or the biological activity of the pharmaceutical composition. Exemplary binders include microcrystalline cellulose, dibasic calcium phosphate, sucrose, corn (maize) starch, modified cellulose (e.g., hydroxymethyl cellulose), or any combination thereof. In one embodiment, the pharmaceutical composition comprises a binder in an amount of about 1 wt % or greater (e.g., about 10 wt % or greater, about 15 wt % or greater, about 20 wt % or greater, or about 25 wt % or greater) by weight of the composition. For example, the pharmaceutical composition comprises from about 5 wt % to about 50 wt % (e.g., from about 10 wt % to about 45 wt % or from about 20 wt % to about 45 wt %) of binder, by weight of the composition. In another example, the pharmaceutical composition comprises about 1 wt % or greater (e.g., about 10 wt % or greater, about 15 wt % or greater, about 20 wt % or greater, or about 22 wt % or greater) of microcrystalline cellulose, by weight of the composition. In yet another example, the pharmaceutical composition comprises from about 5 wt % to about 50 wt % (e.g., from about 10 wt % to about 45 wt % or from about 20 wt % to about 45 wt %) of microcrystalline cellulose, by weight of the composition.

Glidants suitable for the present invention enhance the flow properties of the pharmaceutical composition and are compatible with the ingredients of the pharmaceutical composition, i.e., they do not substantially reduce the solubility, the hardness, the chemical stability, the physical stability, or the biological activity of the pharmaceutical composition. Exemplary glidants include colloidal silicon dioxide, talc, or a combination thereof. In one embodiment, the pharmaceutical composition comprises a glidant in an amount of about 2 wt % or less (e.g., about 1.75 wt % or less, about 1.25 wt % or less, or about 1.00 wt % or less) by weight of the composition. For example, the pharmaceutical composition comprises from about 2 wt % to about 0.05 wt % (e.g., from about 1.5 wt % to about 0.07 wt % or from about 1.0 wt % to about 0.09 wt %) of glidant, by weight of the composition. In another example, the pharmaceutical composition comprises about 2 wt % or less (e.g., about 1.75 wt % or less, about 1.25 wt % or less, or about 1.00 wt % or less) of colloidal silicon dioxide, by weight of the composition. In yet another example, the pharmaceutical composition comprises from about 2 wt % to about 0.05 wt % (e.g., from about 1.5 wt % to about 0.07 wt % or from about 1.0 wt % to about 0.09 wt %) of colloidal silicon dioxide, by weight of the composition.

Lubricants suitable for the present invention improve the compression and ejection of compressed pharmaceutical compositions from a die press and are compatible with the ingredients of the pharmaceutical composition, i.e., they do not substantially reduce the solubility, the hardness, or the biological activity of the pharmaceutical composition. Exemplary lubricants include magnesium stearate, stearic acid (stearin), hydrogenated oil, sodium stearyl fumarate, or any combination thereof. In one embodiment, the pharmaceutical composition comprises a lubricant in an amount of about 2 wt % or less (e.g., about 1.75 wt % or less, about 1.25 wt % or less, or about 1.00 wt % or less) by weight of the composition. For example, the pharmaceutical composition comprises from about 2 wt % to about 0.10 wt % (e.g., from about 1.5 wt % to about 0.15 wt % or from about 1.3 wt % to about 0.30 wt %) of lubricant, by weight of the composition. In another example, the pharmaceutical composition comprises about 2 wt % or less (e.g., about 1.75 wt % or less, about 1.25 wt % or less, or about 1.00 wt % or less) of magnesium stearate, by weight of the composition. In yet another example, the pharmaceutical composition comprises from about 2 wt % to about 0.10 wt % (e.g., from about 1.5 wt % to about 0.15 wt % or from about 1.3 wt % to about 0.30 wt %) of magnesium stearate, by weight of the composition.

In some embodiments, the pharmaceutical composition can optionally comprise one or more colorants, flavors, and/or fragrances to enhance the visual appeal, taste, and/or scent of the composition. Suitable colorants, flavors, or fragrances are compatible with the ingredients of the pharmaceutical composition, i.e., they do not substantially reduce the solubility, the chemical stability, the physical stability, the hardness, or the biological activity of the pharmaceutical composition. In some embodiments, the pharmaceutical composition comprises a colorant, a flavor, and/or a fragrance. For example, the pharmaceutical composition comprises less than about 1 wt % (e.g., less than about 0.75 wt % or less than about 0.5 wt %) of each optionally ingredient, i.e., colorant, flavor and/or fragrance, by weight of the composition. In another example, the pharmaceutical composition comprises less than about 1 wt % (e.g., less than about 0.75 wt % or less than about 0.5 wt %) of a colorant.

In some embodiments, the pharmaceutical composition can comprise tablets and the tablets can be coated with a colorant and optionally labeled with a logo, other image and/or text using a suitable ink. In still other embodiments, the pharmaceutical composition can be made into tablets and the tablets can be coated with a colorant, waxed, and optionally labeled with a logo, other image and/or text using a suitable ink. Suitable colorants and inks are compatible with the ingredients of the pharmaceutical composition, i.e., they do not substantially reduce the solubility, the chemical stability, the physical stability, the hardness, or the biological activity of the pharmaceutical composition. The suitable colorants and inks can be any color and are water based or solvent based. In some embodiments, tablets made from the pharmaceutical composition are coated with a colorant and then labeled with a logo, other image, and/or text using a suitable ink. For example, tablets comprising a pharmaceutical composition as described herein can be coated with about 3 wt % (e.g., less than about 6 wt % or less than about 4 wt %) of film coating comprising a colorant. The colored tablets can be labeled with a logo and text indicating the strength of the active ingredient in the tablet using a suitable ink. The colored tablets can be labeled with a logo and text indicating the strength and/or mass of the active ingredient in the tablet using a black ink (e.g., Opacode® WB, commercially available from Colorcon, Inc. of West Point, Pa.). In another embodiment, tablets made from the pharmaceutical composition are coated with a colorant, waxed, and then labeled with a logo, other image, and/or text using a suitable ink. The colored tablets can be waxed with Carnauba wax powder weighed out in the amount of about 0.01% w/w of the starting tablet core weight. The waxed tablets can be labeled with a logo and text indicating the strength of the active ingredient in the tablet using a suitable ink.

In some embodiments, the pharmaceutical composition consists of a tablet having a hardness of about 5 Kp or greater (e.g., about 5.5 Kp or greater, about 6 Kp or greater, or about 7 Kp or greater).

In some embodiments, the tablet has a dissolution of about 50% or greater in about 30 minutes.

Note that dissolution can be measured with a standard USP Type II apparatus that employs a dissolution media of 0.6% sodium lauryl sulfate dissolved in 900 mL of DI water, stirring at about 50-75 rpm at a temperature of about 37° C. A single experimental tablet is tested in each test vessel of the apparatus. Dissolution can also be measured with a standard USP Type II apparatus that employs a dissolution media of 0.7% sodium lauryl sulfate dissolved in 900 mL of 50 mM sodium phosphate buffer (pH 6.8), stirring at about 65 rpm at a temperature of about 37° C. A single experimental tablet is tested in each test vessel of the apparatus. Dissolution can also be measured with a standard USP Type II apparatus that employs a dissolution media of 0.5% sodium lauryl sulfate dissolved in 900 mL of 50 mM sodium phosphate buffer (pH 6.8), stirring at about 65 rpm at a temperature of about 37° C., wherein a single experimental tablet is tested in each test vessel of the apparatus.

Another aspect of the present invention provides a pharmaceutical composition comprising from about 3 wt % to about 22 wt % of a compound of Formula I; from about 35 wt % to about 45 wt % of a diluent; from about 1 wt % to about 5 wt % of a disintegrant; from about 0.5 wt % to about 2.5 wt % of a wetting agent; from about 35 wt % to about 45 wt % of a binder; from about 0.5 wt % to about 2.5 wt % of a glidant; and from about 0.25 wt % to about 1.0 wt % of a lubricant.

In some embodiments, the pharmaceutical composition further comprises a tablet, a capsule, or a suspension. For example, the pharmaceutical composition comprises a tablet. In some examples, the tablet has a hardness of about 5 Kp or greater (e.g., about 6 Kp or greater).

Another aspect of the present invention provides a pharmaceutical composition consisting of a tablet that comprises a compound of Formula I, a diluent, a disintegrant, a surfactant, a binder, a glidant, and a lubricant, wherein the tablet has a dissolution of about 50% or greater in about 30 minutes.

Another embodiment provides a pharmaceutical composition consisting of a tablet that comprises a compound of Formula I; a diluent; a disintegrant; a surfactant; a binder; a glidant; and a lubricant.

One embodiment provides a pharmaceutical composition comprising:
a. about 20 wt % of a compound of Formula I by weight of the composition;
b. about 37 wt % of microcrystalline cellulose by weight of the composition;
c. about 37 wt % of lactose by weight of the composition;
d. about 3 wt % of sodium croscarmellose by weight of the composition;
e. about 1 wt % of SLS by weight of the composition;
f. about 1 wt % of colloidal silicon dioxide by weight of the composition; and
g. about 0.75 wt % of magnesium stearate by weight of the composition.

Another embodiment provides a pharmaceutical composition comprising:
a. about 10 wt % of a compound of Formula I by weight of the composition;
b. about 47 wt % of microcrystalline cellulose by weight of the composition;
c. about 47 wt % of lactose by weight of the composition;
d. about 3 wt % of sodium croscarmellose by weight of the composition;
e. about 1 wt % of SLS by weight of the composition;
f. about 1 wt % of colloidal silicon dioxide by weight of the composition; and
g. about 0.75 wt % of magnesium stearate by weight of the composition.

### IV. METHODS OF PRODUCING A PHARMACEUTICAL COMPOSITION

Another aspect of the present invention provides a method of producing a pharmaceutical composition comprising providing an admixture comprising:

a. a compound of Formula I

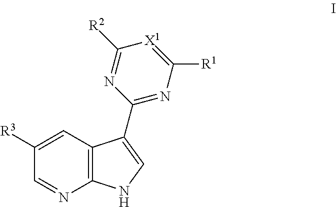

or a pharmaceutically acceptable salt thereof, wherein:

X¹ is N or CR⁴;

R² is H or halo;

R³ is H or halo;

R⁴ is H or halo;

R¹ is

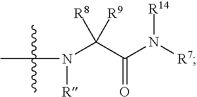

R″ is H or an unsubstituted C₁₋₂ aliphatic;

R⁸ is an unsubstituted C₁₋₄ aliphatic;

R⁹ is an unsubstituted C₁₋₄ aliphatic

R⁷ is a C₁₋₃ aliphatic optionally substituted with up to 3 occurrences of F; and

R¹⁴ is H or unsubstituted C₁₋₂ alkyl;

b. a diluent;

c. a disintegrant;

d. a wetting agent;

e. a binder;

f. a glidant; and

g. a lubricant, and

compressing the admixture into a tablet.

Another aspect of the present invention provides a method of producing a pharmaceutical composition comprising providing an admixture comprising:

a. a compound selected from Table 1;

b. a binder;

c. a glidant;

d. a surfactant;

e. a lubricant;

f. a disintegrant; and

g. a diluent, and

compressing the admixture into a tablet.

In several embodiments, the admixture is compressed to produce a tablet having a hardness of 5 Kp or greater. In others, the admixture is compressed to produce a tablet having a dissolution of about 50% or greater in about 30 minutes. Several examples also include the step of mixing the admixture until the admixture is substantially homogenous.

Each of the ingredients of this admixture is described above and in the Examples below. Furthermore, the admixture can comprise optional additives such as one or more colorants, one or more flavors, and/or one or more fragrances as described above and in the Examples below. And, the relative concentrations (e.g., wt %) of each of these ingredients (and any optional additives) in the admixture is also presented above and in the Examples below. The ingredients constituting the admixture can be provided sequentially or in any combination of additions; and, the ingredients or combination of ingredients can be provided in any order. In one embodiment the lubricant is the last component added to the admixture.

In some embodiments, the pharmaceutical composition comprises a compound of Formula I, a binder, a glidant, a surfactant, a lubricant, a disintegrant, and a filler, wherein each of these ingredients comprises a powder (e.g., provided as particles having a mean diameter, measured by light scattering, of about 250 μm or less (e.g., about 150 μm or less, about 100 μm or less, about 50 μm or less, about 45 μm or less, about 40 μm or less, or about 35 μm or less)). For instance, the pharmaceutical composition comprises a compound of Formula I, wherein the compound of Formula I comprises a powder having a mean diameter of about 250 μm or less (e.g., about 150 μm or less, about 100 μm or less, about 50 μm or less, about 45 μm or less, about 40 μm or less, or about 35 μm or less). In other instances, the pharmaceutical composition comprises one or more excipients selected from a binder, a glidant, a surfactant, a lubricant, a disintegrant, and a filler, wherein the excipient comprises a powder having a mean particle diameter of about 250 μm or less (e.g., about 150 μm or less, about 100 μm or less, about 50 μm or less, about 45 μm or less, about 40 μm or less, or about 35 μm or less).

In one embodiment, the admixture comprises a compound of Formula I, a binder, a glidant, a surfactant, a lubricant, a disintegrant, and a filler, wherein each of these ingredients is provided in a powder form (e.g., provided as particles having a mean diameter, measured by light scattering, of about 250 μm or less (e.g., about 150 μm or less, about 100 μm or less, about 50 μm or less, about 45 μm or less, about 40 μm or less, or about 35 μm or less)). For instance, the admixture comprises a compound of Formula I, a binder, a glidant, a surfactant, a lubricant, a disintegrant, and a filler, wherein each of these ingredients is provided in a powder form (e.g., provided as particles having a mean diameter, measured by light scattering, of about 250 μm or less (e.g., about 150 μm or less, about 100 μm or less, about 50 μm or less, about 45 μm or less, about 40 μm or less, or about 35 μm or less)).

In another embodiment, the admixture comprises a compound of Formula I, a binder, a glidant, a surfactant, a lubricant, a disintegrant, and a filler, wherein each of these ingredients is substantially free of water. In these embodiments, each of the ingredients comprises less than about 5 wt % (e.g., less than about 2 wt %, less than about 1 wt %, less than about 0.75 wt %, less than about 0.5 wt %, or less than about 0.25 wt %) of water by weight of the ingredient.

In another embodiment, compressing the admixture into a tablet is accomplished by filling a form (e.g., a mold) with the admixture and applying pressure to admixture. This can be accomplished using a die press or other similar apparatus. It is also noted that the application of pressure to the admixture in the form can be repeated using the same pressure during each compression or using different pressures during the plurality of compressions. In another example, the admixture is compressed using a die press that applies sufficient pressure to form a tablet having a dissolution of about 50% or more at about 30 minutes (e.g., about 55% or more at about 30 minutes or about 60% or more at about 30 minutes). For instance, the admixture is compressed using a die press to produce a tablet hardness of about 5 Kp or greater (about 5.5 Kp or greater, about 6 Kp or greater, about 7 Kp or greater, about 11 Kp or greater, or about 21 Kp or greater). In some instances, the admixture is compressed to produce a tablet hardness of between about 6 and about 21 Kp.

In some embodiments, tablets comprising a pharmaceutical composition as described herein can be coated with about 3.0 wt % of a film coating comprising a colorant by weight of the tablet. In certain instances, the colorant suspension or solution used to coat the tablets comprises about 20% w/w of solids by weight of the colorant suspension or solution. In still further instances, the coated tablets can be labeled with a logo, other image or text.

In another embodiment, the method of producing a pharmaceutical composition comprises providing an admixture of a compound of Formula I, a binder, a glidant, a surfactant, a lubricant, a disintegrant, and a filler; mixing the admixture until the admixture is substantially homogenous, and compressing the admixture into a tablet as described above or in the Examples below. Or, the method of producing a pharmaceutical composition comprises providing an admixture of a compound of Formula I, a binder, a glidant, a surfactant, a lubricant, a disintegrant, and a filler; mixing the admixture until the admixture is substantially homogenous, and compressing the admixture into a tablet as described above or in the Examples below. For example, the admixture is mixed by stirring, blending, shaking, or the like using hand mixing, a mixer, a blender, any combination thereof, or the like. When ingredients or combinations of ingredients are added sequentially, mixing can occur between successive additions, continuously throughout the ingredient addition, after the addition of all of the ingredients or combinations of ingredients, or any combination thereof. The admixture is mixed until it has a substantially homogenous composition.

### V. ADMINISTRATION OF A PHARMACEUTICAL FORMULATION

In another aspect, the invention also provides a method of treating or lessening the severity of a disease selected from spondyloarthropathy (e.g., peripheral spondyloarthropathy, axial spondyloarthropathy, reactive arthritis, Reiter's syndrome, psoriatic arthritis, ankylosing spondylitis, ulcerative colitis, Crohn's disease, or any combination thereof), systemic lupus erythematosus, rheumatoid arthritis (RA), or any combination thereof or any combination thereof in a patient comprising administering to said patient one of the compositions as defined herein optionally in combination with a chemotherapy agent, a DMARD, or any combination thereof.

Another aspect of the present invention provides a method of administering a pharmaceutical composition by orally administering to a patient at least once per day the composition comprising about 20 mg or greater (e.g., about 25 mg) of a compound of Formula I.

Another aspect of the present invention provides a method of administering a pharmaceutical composition by orally administering to a patient at least once per day the composition comprising about 45 mg or greater (e.g., about 50 mg) of a compound of Formula I.

Another aspect of the present invention provides a method of administering a pharmaceutical composition comprising orally administering to a patient at least once per day at least one tablet comprising a pharmaceutical composition comprising:
a. a compound of Formula I;
b. a diluent;
c. a binder;
d. a glidant;
e. a disintegrant;
f. a surfactant; and
g. a lubricant.

In several embodiments, the tablet comprising the pharmaceutical composition comprising a compound of Formula I, the diluent, the binder, the glidant, the disintegrant, the surfactant, and the lubricant is orally administered to the patient once per day or about every 12 hours. In other embodiments, the tablet comprising the pharmaceutical composition comprising a compound of Formula I, the diluent, the binder, the glidant, the disintegrant, the surfactant, and the lubricant is orally administered to the patient twice per day.

In some embodiments, the tablet comprises about 25 mg or greater of a compound of Formula I.

In some instances, the tablet comprises about 25 mg of a compound of Formula I. In other instances, the tablet comprises about 50 mg of a compound of Formula I.

Another aspect of the present invention provides a method of administering a pharmaceutical composition by orally administering to a patient at least once per day the composition comprising about 20 mg or greater of a compound of Formula I.

Another aspect of the present invention provides a method of administering a pharmaceutical composition by orally administering to a patient at least once per day the composition comprising about 25 mg of a compound of Formula I.

Another aspect of the present invention provides a method of administering a pharmaceutical composition by orally administering to a patient at least once per day the composition comprising about 50 mg of a compound of Formula I.

Another aspect of the present invention provides a method of administering a pharmaceutical composition by orally administering to a patient twice per day the composition comprising a compound of Formula I.

Another aspect of the present invention provides a method of administering a pharmaceutical composition by orally administering to a patient twice per day the composition comprising about 25 mg of a compound of Formula I.

Another aspect of the present invention provides a method of administering a pharmaceutical composition by orally administering to a patient twice per day the composition comprising about 50 mg of a compound of Formula I.

Another aspect of the present invention provides a method of administering a pharmaceutical composition by orally administering to a patient twice per day the composition comprising about 100 mg of a compound of Formula I.

Another aspect of the present invention provides a method of administering a pharmaceutical composition by orally administering to a patient twice per day the composition comprising about 150 mg of a compound of Formula I.

Another aspect of the present invention provides a method of administering a pharmaceutical composition by orally administering to a patient a pharmaceutical composition once every 12 hours. The composition comprises about 25 mg or greater of a compound of Formula I.

Another aspect of the present invention provides a method of administering a pharmaceutical composition by orally administering to a patient once every 12 hours, about 50 mg or greater of a compound of Formula I.

Another aspect of the present invention provides a method of administering a pharmaceutical composition by orally administering to a patient once every 12 hours, about 100 mg or greater of a compound of Formula I.

Another aspect of the present invention provides a method of administering a pharmaceutical composition by orally administering to a patient once every 12 hours, about 150 mg or greater of a compound of Formula I.

In still other aspects of the present invention, a pharmaceutical composition as described herein is orally administered to a patient once every 24 hours.

Another aspect of the present invention provides a method of administering a pharmaceutical composition comprising orally administering to a patient at least once per day at least one tablet comprising a pharmaceutical composition comprising a compound of Formula I, a diluent, a binder, a glidant, a disintegrant, a surfactant, and a lubricant, each of which is described above and in the Examples below, wherein the composition comprises about 25 mg or greater (e.g., at least about 35 mg, at least about 40 mg, or at least about 45 mg) of a compound of Formula I.

In some embodiments, the present invention provides a method of administering a pharmaceutical composition comprising orally administering to a patient at least one tablet comprising:
a. about 25 mg of a compound of Formula I;
b. a diluent;
c. a disintegrant;
d. a wetting agent;
e. a binder;
f. a glidant; and
g. a lubricant.

In some embodiments, the present invention provides a method of administering a pharmaceutical composition comprising orally administering to a patient at least one tablet comprising:
a. about 50 mg of a compound of Formula I;
b. a diluent;
c. a disintegrant;
d. a surfactant;
e. a binder;
f. a glidant; and
g. a lubricant.

In some embodiments, the present invention provides for a method of orally administering the pharmaceutical composition described herein once a day. In other embodiments, the present invention provides for a method of orally administering the pharmaceutical composition described herein twice a day.

Another aspect of the present invention provides a method of administering a pharmaceutical composition by orally administering to a patient at least once per day at least one tablet comprising about 25 mg or greater of a compound of Formula I, a diluent, a binder, a glidant, a disintegrant, a surfactant, and a lubricant. In some embodiments, the tablet is orally administered to the patient once per day. In other embodiments, the administration comprises orally administering to a patient twice per day at least one tablet comprising about 25 mg or greater of a compound of Formula I, a diluent, a binder, a glidant, a disintegrant, a surfactant, and a lubricant. Some tablets useful in this method comprise about 50 mg of a compound of Formula I. In another method, the administration includes orally administering to a patient twice per day at least one tablet comprising about 50 mg or greater of a compound of Formula I, a diluent, a binder, a glidant, a disintegrant, a surfactant, and a lubricant.

In one embodiment, the method of administering a pharmaceutical composition includes orally administering to a patient at least once per day at least one tablet comprising a pharmaceutical composition containing from about 20 mg to about 55 mg of a compound of Formula I; and a diluent, a binder, a glidant, a disintegrant, a surfactant, and a lubricant.

In another embodiment, the method of administering a pharmaceutical composition includes orally administering to a patient once per day at least one tablet comprising a pharmaceutical composition containing a compound of Formula I, a filler, a binder, a glidant, a disintegrant, a surfactant, and a lubricant, each of which is described above and in the Examples below, wherein the compound of Formula I is present in an amount of about 25 mg or greater (e.g., about 35 mg or greater, about 40 mg or greater, about 45 mg or greater, about 75 mg or greater, about 100 mg or greater, about 150 mg or greater, or about 250 mg or greater). In another example, the method of administering a pharmaceutical composition includes orally administering to a patient once per day a plurality of tablets (e.g., two tablets, three tablets, four or five tablets), wherein each tablet comprises a pharmaceutical composition comprising a compound of Formula I, a filler, a binder, a glidant, a disintegrant, a surfactant, and a lubricant, wherein the compound of Formula I is present in an amount of about 25 mg or greater (e.g., about 35 mg or greater, about 40 mg or greater, about 45 mg or greater, about 75 mg or greater, about 150 mg or greater, or about 250 mg or greater).

In another embodiment, the method of administering a pharmaceutical composition includes orally administering to a patient twice per day at least one tablet comprising a pharmaceutical composition containing a compound of Formula I, a filler, a binder, a glidant, a disintegrant, a surfactant, and a lubricant, each of which is described above and in the Examples below, wherein the compound of Formula I is present in an amount of about 25 mg or greater (e.g., about 35 mg or greater, about 40 mg or greater, about 45 mg or greater, about 50 mg or greater, about 75 mg or greater, about 150 mg or greater, or about 250 mg or greater). In another example, the method of administering a pharmaceutical composition includes orally administering to a patient twice per day a plurality of tablets (e.g., two tablets, three tablets, four tablets or five tablets), wherein each tablet comprises a pharmaceutical composition comprising a compound of Formula I, a filler, a binder, a glidant, a disintegrant, a surfactant, and a lubricant, wherein the compound of Formula I is present in an amount of about 20 mg or greater (e.g., about 25 mg or greater, about 30 mg or greater, about 35 mg or greater, about 45 mg or greater, about 75 mg or greater, about 150 mg or greater, or about 250 mg or greater) per tablet. In embodiments wherein a plurality of tablets are administered, each of the tablets may comprise about the same amount of a compound of Formula I or at least two of the tablets may comprise different amounts of the compound of Formula I.

It will also be appreciated that the compound and pharmaceutically acceptable compositions of the present invention can be employed in combination therapies, that is, the compound and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated”.

In one embodiment, the additional agent is selected from a mucolytic agent, bronchodialator, an anti-biotic, an anti-infective agent, an anti-inflammatory agent, a protein kinase inhibitor other than a compound of Formula I, or a nutritional agent (e.g., nutraceutical or vitamin).

In another embodiment, the additional agent is an anti-inflammatory agent, i.e., an agent that can reduce the inflammation in the lungs. Exemplary such agents useful herein include ibuprofen, docosahexanoic acid (DHA), sildenafil, inhaled glutathione, pioglitazone, hydroxychloroquine, or simavastatin.

In another embodiment, the additional agent is a nutritional agent. Exemplary agents include vitamin a, vitamin b, vitamin c, vitamin e, pancrelipase (pancreating enzyme replacement), including Pancrease®, Pancreacarb®, Ultrase®, or Creon®, Liprotomase® (formerly Trizytek®), Aquadeks®, or glutathione inhalation. In one embodiment, the additional nutritional agent is pancrelipase.

### VI. EXAMPLES

In order that the invention described herein may be more fully understood, the following examples are set forth. It should be understood that these examples are for illustrative purposes only and are not to be construed as limiting this invention in any manner.

### Example 1

### Analytical Methods Used

(A) HPLC on C18 column. Mobile phase was acetonitrile/water/TFA (60:40:0.1). Flow rate was 1.0 mL/min. Detection at wavelength of 230 nm. Run time was 25-26 minutes.

(B) HPLC on C18 column. Mobile phase was acetonitrile/water/TFA (90:10:0.1). Flow rate was 1.0 mL/min. Detection at wavelength of 230 nm.

(C) HPLC on a Waters XBridge Phenyl column, 4.6×150 mm, 3.5 μm. Mobile phase A was water/1M ammonium formate, pH 4.0 (99:1). Mobile phase B was acetonitrile/water/1M ammonium formate, pH 4.0 (90:9:1). Gradient 5% to 90% B in 15 minutes. Total run time 22 minutes. Flow rate 1.5 mL/min. Detection at UV, 245 nm. T=25° C.

(D) HPLC on a Waters XBridge Phenyl column, 4.6×150 mm, 3.5 μm. Mobile phase A was water/1M ammonium formate, pH 4.0 (99:1). Mobile phase B was acetonitrile/water/1M ammonium formate, pH 4.0 (90:9:1). Gradient 15% to 90% B in 15 minutes. Total run time 22 minutes. Flow rate 1.5 mL/min. Detection at UV, 220 nm. T=35° C.

### Example 2

### Preparation of Compounds of Formula I

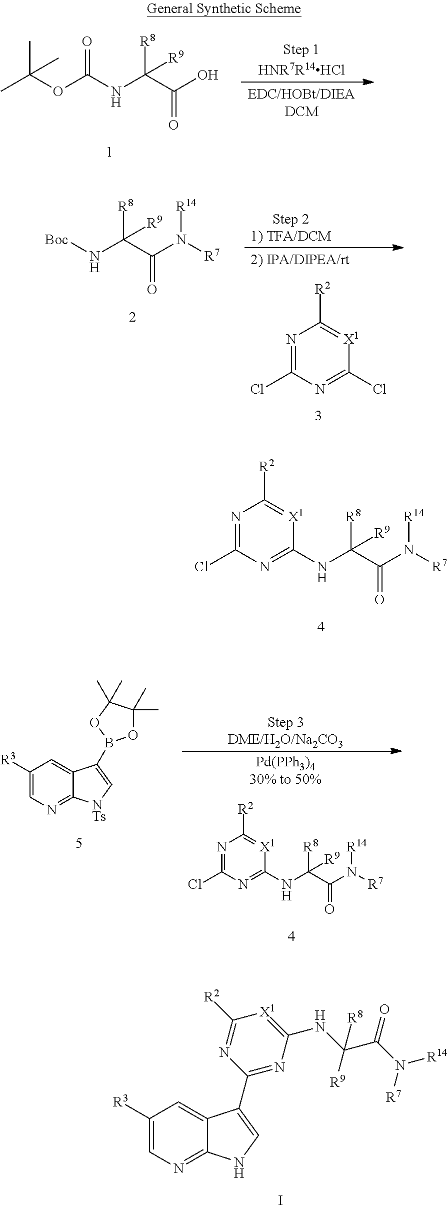

The Boc-protected amino acid starting material (1) undergoes amidation in the presence of an activating agent, a coupling reagent, and the acid salt of the amine HNR⁷R⁷ to generate the Boc-protected amide intermediate (2). The amide intermediate (2) is deprotected under acidic conditions and reacted with the halogenated heteroaryl (3) to generate the aminoheteroaryl intermediate (4). Boronated azaindole (5) is coupled with the aminoheteroaryl intermediate (4) under cross-coupling condition to generate the compound of Formula I.

### Example 3

### Synthesis of 2-(2-(1H-pyrrolo[2,3-b]pyridin-3-yl)pyrimidin-4-ylamino)-2-methyl-N-(2,2,2-trifluoroethyl)butanamide

### 3-bromo-1H-pyrrolo[2,3-b]pyridine

Commercially available 7-Azaindole (6.9 kg, 58.4 moles) was added to a 200 L glass-lined reactor containing 52.6 kg DMF. A solution of Br₂ in DMF (9.7 kg Br₂ in 14.7 kg DMF) was added drop wise to maintain the mixture temperature of about 0-10° C. After the addition was complete, the temperature was maintained at about 0-10° C. The completeness of the reaction was measured by HPLC (method A) with sample aliquots after 30 minutes. The reaction was considered complete when the 7-azaindole was less than 3% (after about 2 hours and 40 minutes). Typical retention time for 3-bromo-1H-pyrrolo[2,3-b]pyridine was 3.228 minutes.

The reaction was quenched with 10% aqueous solution of NaHSO₃ (17.5 kg) while maintaining the temperature below 15° C. A saturated aqueous solution of NaHCO₃ (61.6 kg) below 25° C. was added to adjust the pH to about 7 to 8. After neutralization, the mixture was transferred into a 50 L vacuum filter and filtered. The resultant cake was washed with water (18 kg) and then petroleum ether (12 kg). The cake was dried in a tray dryer at about 50-60° C. until the water content detected by KF (Karl Fisher reaction) was less than 0.8%. A yellow solid resulted (10.3 kg, 99.1% purity as measured by HPLC (method A), 89.6% yield of 3-bromo-1H-pyrrolo[2,3-b]pyridine).

### 3-bromo-1-tosyl-1H-pyrrolo[2,3-b]pyridine

3-bromo-1H-pyrrolo[2,3-b]pyridine (10.7 kg, 54.3 moles) was added to 94.3 kg of THF in a 200 L glass-lined reactor. The solid was dissolved completely by stirring. After the mixture was cooled to about 10-15° C., NaH (3.4 kg, 85 moles) was added in portions (about 200-250 g each portion) every 3 to 5 minutes while venting any H₂ gas released by the reaction. After the addition of NaH, the mixture was stirred for one hour while maintaining the temperature of about 10-20° C. 4-methylbenzenesulfonylchloride (12.4 kg, 65.0 moles) was added at a rate of 0.5 kg/10 minutes at about 10-20° C. After the addition was complete, the temperature was maintained at about 10-20° C. The completeness of the reaction was measured by HPLC (method A) with sample aliquots after 30 minutes. The reaction was considered complete when the peak area of 3-bromo-1H-pyrrolo[2,3-b]pyridine was less than 1% (after about 1.5 hours). Typical retention time for 3-bromo-1-tosyl-1H-pyrrolo[2,3-b]pyridine was 20.2 minutes.

The reaction was quenched with water (10.7 kg) while maintaining the temperature below 20° C. Dichloromethane (41.3 kg) was added to the mixture. Then 3% HCl acid (42.8 kg) was added into the mixture while maintaining the temperature below 25° C. After the addition, the phases were allowed to separate for 0.5 hour. The aqueous phase was extracted twice with dichloromethane. During each extraction, the mixture was stirred for 15 minutes and then held for 15 minutes. All the organic phases were combined. The combined organic phases were washed with 3% HCl acid (33.4 kg) and water (40 kg). During each wash, the mixture was stirred for 15 minutes and then held for 30 minutes.

The mixture was transferred into a 50 L vacuum filter and filtered through silica gel (3 kg). The cake was washed with dichloromethane (35 kg) twice. The filtrate and washings were combined. The organic phase was concentrated below 40° C. under vacuum of a pressure less than −0.085 MPa until 10 L mixture remained. Petroleum ether (9 kg) was added into the residue. The mixture was stirred until it was homogeneous. The slurry was transferred into a 50 L vacuum filter and filtered. The cake was washed with petroleum ether (9 kg). A light brown solid resulted (17 kg, 99.7% purity as measured by HPLC analysis (method A), 94% yield of 3-bromo-1-tosyl-1H-pyrrolo[2,3-b]pyridine).

### 1-tosyl-1H-pyrrolo[2,3-b]pyridin-3-ylboronic acid

THF (28.5 kg) and 3-bromo-1-tosyl-1H-pyrrolo[2,3-b]pyridine (4 kg) were added to a 72 L flask. The mixture was stirred until the solid dissolved completely. Triisopropyl borate (3.2 kg) was added and the mixture was cooled to below −80° C. n-BuLi (4.65 kg) was added drop wise at a rate of about 0.6-0.9 kg/hour maintaining the temperature of about −80 to −90° C. After the addition, the temperature was maintained at about −80 to −90° C. The completeness of the reaction was measured by HPLC (method A) with sample aliquots after 30 minutes. The reaction was considered complete when the peak area of 3-bromo-1-tosyl-1H-pyrrolo[2,3-b]pyridine was less than 4%. Typical retention time for 1-tosyl-1H-pyrrolo[2,3-b]pyridin-3-ylboronic acid was 4.6 minutes. Extra triisopropyl borate and n-BuLi was added to lower the peak area of 3-bromo-1-tosyl-1H-pyrrolo[2,3-b]pyridine.

Water (2 kg) was slowly added to the mixture to quench the reaction. The mixture temperature returned to about 15-25° C. The mixture was transferred to a 50 L reactor to be concentrated below 40° C. under vacuum of a pressure less than −0.08 MPa until no THF distilled out. The residue was dissolved into water (25 kg) and 10% aqueous NaOH solution (26 kg). The mixture was stirred until the solid dissolved completely. The mixture was transferred into a vacuum filter and filtered. The filtrate was extracted twice with MTBE (21 kg each) at about 20-30° C. During each extraction, the mixture was stirred 15 minutes and held 15 minutes. HCl acid (28 L) was added into the aqueous phase to adjust the pH to between 3 and 4 while maintaining the temperature of about 10-20° C. The mixture was stirred at about 10-15° C. for 1 hour. The mixture was transferred into a centrifuge and filtered. The resultant cake after filtering was washed with water (5 kg) and petroleum ether (5 kg). The cake was dried at 35-45° C. until the LOD (loss on drying) was less than 3%. An off-white solid resulted (2.5 kg and 98.8% purity as measured by HPLC analysis (method A), 69.4% yield of 1-tosyl-1H-pyrrolo[2,3-b]pyridin-3-ylboronic acid).

### 3-(4,4,5,5-tetramethyl-1,3,2-dioxaborolan-2-yl)-1-tosyl-1H-pyrrolo[2,3-b]pyridine

Dichloromethane (165.6 kg) and pinacolate alcohol (3.54 kg) was added to a 200 L glass-lined reactor. The mixture was stirred until the solid dissolved completely. Then, 1-tosyl-1H-pyrrolo[2,3-b]pyridin-3-ylboronic acid (8.65 kg) was added in portions (2 kg every 5 minutes) while maintaining the temperature of about 20-30° C. After the addition, the temperature was maintained at about 20-30° C. while stirring. The completeness of the reaction was measured by HPLC (method B) with sample aliquots every 60 minutes. The reaction was considered complete when the peak area of 31-tosyl-1H-pyrrolo[2,3-b]pyridin-3-ylboronic acid was less than 1%. Typical retention time for 3-(4,4,5,5-tetramethyl-1,3,2-dioxaborolan-2-yl)-1-tosyl-1H-pyrrolo[2,3-b]pyridine) was 6.4 minutes.

The mixture was filtered through silica gel (3 kg). The cake was rinsed twice with dichloromethane (15 kg each rinse). The filtrate was combined with the washing liquids, and then concentrated below 30° C. under vacuum at a pressure less than −0.08 MPa until no fraction distilled out. Solvent was continued to be removed by vacuum for 2 hours. Isopropanol (17.2 kg) was added to the residue. The mixture was heated to reflux at about 80-85° C. The mixture refluxed for 30 minutes until the solid dissolved completely. The mixture was cooled below 35° C., and then to about 0-10° C. The mixture crystallized at 0-10° C. for 2 hours and then filtered. After filtration, the resultant cake was dried at about 35-45° C. until the water content detected by KF (Karl Fisher reaction) was less than 0.5% and the LOD (loss on drying) was less than 0.5%. An off-white solid resulted (8.8 kg and 99.7% purity as measured by HPLC analysis (method B), 81.5% yield of 3-(4,4,5,5-tetramethyl-1,3,2-dioxaborolan-2-yl)-1-tosyl-1H-pyrrolo[2,3-b]pyridine).

### (R)-2-methyl-2-(2-(1-tosyl-1H-pyrrolo[2,3-b]pyridin-3-yl)pyrimidin-4-ylamino)butanoic acid

Tripotassium phosphate (K₃PO₄) (7.20 kg, 3 equiv.) was mixed with three volumes of water (9.0 kg). The mixture was agitated for at least 20 minutes, cooled to a temperature of ≦30° C. and added to acetonitrile (16.8 g, 7 volumes) into a 120 L reactor. The resultant mixture was agitated. 3.0 kg (11.3 moles, 1.0 equiv.) of (R)-2-(2-chloropyrimidin-4-ylamino)-2-methylbutanoic acid hydrochloride were added to the reaction mixture in the reactor while maintaining a temperature ≦30° C. The mixture was agitated for at least 20 minutes. 5.16 kg (13.0 moles, 1.15 equiv.) of 3-(4,4,5,5-tetramethyl-1,3,2-dioxaborolan-2-yl)-1-tosyl-1H-pyrrolo[2,3-b]pyridine were then added to the reactor. The reaction mixture was agitated and de-gassed with N₂ sparging for at least 30 minutes. The mixture was heated to 65±5° C.

In a separate vessel, 0.075 kg (0.03 equiv.) of palladium(II) acetate was mixed with 4.80 kg (2 volumes) of de-gassed acetonitrile (CH₃CN). This mixture was agitated until homogenous. 0.267 kg (1.02 moles, 0.09 equiv.) of triphenylphosphine (PPh₃) was added and the resultant mixture was agitated for at least 30 minutes at 20±5° C. The palladium(II) acetate/PPh3/CH₃CN mixture was then added to the reactor above while maintaining the nitrogen purge. The reactor contents were heated to 75±5° C. for at least 17 hours under nitrogen purge. After 5 hours the conversion was shown to be about 86% complete as measured by HPLC analysis (method C) of a 1.0 mL aliquot. Typical retention times are 6.2 minutes for (R)-2-(2-chloropyrimidin-4-ylamino)-2-methylbutanoic acid hydrochloride and 10.6 minutes for (R)-2-methyl-2-(2-(1-tosyl-1H-pyrrolo[2,3-b]pyridin-3-yl)pyrimidin-4-ylamino)butanoic acid. Additional catalyst and 3-(4,4,5,5-tetramethyl-1,3,2-dioxaborolan-2-yl)-1-tosyl-1H-pyrrolo[2,3-b]pyridine (900 g, 2.26 moles, 0.2 equiv.) were then added to the reaction mixture and the mixture was stirred. After an additional 12 hours, the reaction was shown to be 99.7% complete as measured by HPLC analysis (method C) of a 1.0 mL aliquot. The additional catalyst added above was prepared by dissolving 37.5 g Palladium(II) acetate in 1 volume of acetonitrile (which was de-gassed for 20 minutes), and then adding 133.5 g of triphenylphosphine.

### (R)-2-(2-(1H-pyrrolo[2,3-b]pyridin-3-yl)pyrimidin-4-ylamino)-2-methylbutanoic acid

A solution of 4N aqueous KOH, which was previously prepared with 6.0 kg of KOH in 27.0 kg of water at a rate to control the temperature rise, was added to the reactor above and the reaction was heated to 75±5° C. for at least 5 hours while agitating the mixture. An aliquot of about 1.0 mL was removed from the reaction mixture and analyzed by HPLC (method C) to show 98.6% R)-2-(2-(1H-pyrrolo[2,3-b]pyridin-3-yl)pyrimidin-4-ylamino)-2-methylbutanoic acid and 1.4% (R)-2-methyl-2-(2-(1-tosyl-1H-pyrrolo[2,3-b]pyridin-3-yl)pyrimidin-4-ylamino)butanoic acid. Typical retention times are 10.6 minutes for (R)-2-methyl-2-(2-(1-tosyl-1H-pyrrolo[2,3-b]pyridin-3-yl)pyrimidin-4-ylamino)butanoic acid and 5.5 minutes for (R)-2-(2-(1H-pyrrolo[2,3-b]pyridin-3-yl)pyrimidin-4-ylamino)-2-methylbutanoic acid.

15.0 kg (5 volumes) of water was added to the reactor. The reaction mixture was cooled to 35±5° C. Isopropyl acetate (7.8 g, 3 volumes) was added, and the reaction mixture was agitated for at least 5 minutes. The reaction mixture was filtered through a 4-cm pad of celite in an 18-inch Nutsche filter. The reactor was rinsed with 9.0 kg of water and the water was then used to rinse the celite pad. The aqueous and organic phases were separated. 0.9 kg of Darco G-60 activated carbon (30% w/w) was added to the aqueous phase in a 120-liter reactor. The pH of the mixture was adjusted to less than 1.0 with concentrated HCl solution at 25±10° C. and held for at least 4 hours. If necessary, the pH was readjusted with 6N NaOH. The mixture was then filtered through a Nutshce filter, which was equipped with a filter cloth, and the solids were rinsed with 6.0 kg (2 volumes) of 1N HCl. The filter cake was maintained under positive pressure of nitrogen for at least 30 minutes. The HCl filtrate was agitated and heated to 25±5° C. 0.9 kg of Darco G-60 activated carbon was added to the HCl filtrate and the mixture was stirred for at least 4 hours. The mixture was then filtered through a Nutshce filter, which was equipped with a filter cloth, and the solids were washed with 6.0 kg (2 volumes) of 1N HCl. The second filter cake was maintained under positive pressure of nitrogen for at least 30 minutes. The HCl filtrate was again agitated and heated, charcoal was added and filtering step was repeated with a Nutshce filter, which was equipped with a 0.45 um in-line filter between the Nutsche filter and the receiver flask, to yield a third filter cake and a final filtrate. The solids were washed with 6.0 kg of 1N HCl. The third filter cake was maintained under positive pressure of nitrogen for at least 30 minutes.

The pH of the final filtrate was adjusted to between 4.5 and 5.0 using 6N NaOH while the temperature was maintained between 25±5° C. If necessary, the pH was readjusted using 1N HCl. The final filtrate was then cooled to 5±5° C. and agitated for at least 2 hours. The mixture was filtered was filtered with a Nutshce filter, which was equipped with a filter cloth. The solids were rinsed with 6.0 kg (2 volumes) of water. The final filter cake was maintained under positive pressure of nitrogen for at least 30 minutes.

The wet solids (i.e., filter cakes) were dried in a drying oven at ≦60° C. under vacuum, with a nitrogen purge, over 5 days to yield 3.561 kg of (R)-2-(2-(1H-pyrrolo[2,3-b]pyridin-3-yl)pyrimidin-4-ylamino)-2-methylbutanoic acid (yield of 102%).

### 2-(2-(1H-pyrrolo[2,3-b]pyridin-3-yl)pyrimidin-4-ylamino)-2-methyl-N-(2,2,2-trifluoroethyl)butanamide

Diisopropylethylamine (DIEA) (3.61 kg, 28.1 moles, 2.5 equiv.) was added to (R)-2-(2-(1H-pyrrolo[2,3-b]pyridin-3-yl)pyrimidin-4-ylamino)-2-methylbutanoic acid (3.5 kg, 11.24 moles, 1.0 equiv.) in 7 volumes (32.6 kg) of dichloromethane (CH₂Cl₂ or DCM) while keeping the temperature at ≦30° C. Water (0.103 kg) was added to make 5.5±0.5% total water content for the reaction system, and the mixture was stirred at ≦30° C. for at least 30 minutes. The reaction mixture was cooled to 0±5° C. Propylphosphonic anhydride solution (17.9 kg, 28.1 moles, 2.5 equiv.) was added to the mixture while maintaining the temperature below 20° C. The mixture was agitated for at least an hour keeping the temperature at 20±5° C., then 2,2,2-trifluoroethylamine (1.68 kg, 16.86 moles, 1.5 equiv.) was added while maintaining the temperature below 20° C. The reaction mixture was warmed to 25±5° C. and agitated for 5 hours while holding the temperature. A 1.0 mL aliquot was removed and the reaction was determined to be 100% complete. Water (17.5 kg, 5 volumes) was added to the reaction mixture, and the resultant mixture was agitated for at least 30 minutes while maintaining the temperature below 30° C.

The mixture was concentrated under vacuum with a rotary evaporator at a temperature ≦45° C. Isopropylacetate (1.55 kg, 0.5 volumes) was added to the concentrated aqueous solution, and the pH of the solution was adjusted to 7.5-8.0 using 6N NaOH solution at ≦35° C. The mixture was cooled to 10±5° C. and stirred at for at least one hour. If necessary, 6N HCl was added to readjust the pH of mixture to 7.5-8.0. The resultant slurry was filtered and washed with water (10.5 kg, 3 volumes). The filter cake was maintained under positive pressure of nitrogen for at least 30 minutes. The wet cake was dissolved in methanol (44.7 kg, 12 volumes) by agitation, and the solution was treated with PL-BnSH MP-Resin (BNSHMP) polymer resin (0.235 kg of 5 wt of resin) at 25±5° C. After agitating at 25±5° C. for at least 12 hours, the mixture was filtered. The solids were washed with methanol (2.77 kg, 1 volume). The filtrate was concentrated under vacuum in a rotary evaporator at a temperature ≦50° C. The filtrate was not concentrated to dryness. The concentrated filtrate was allowed to sit at room temperature for about 2.5 days. The mixture was then stirred until homogeneous and heated to 40° C., followed by slow addition of pre-heated water (56.1 kg at 45° C.) while maintaining a temperature of 45±5° C. After the mixture was spun for 1 hour, the remaining methanol was concentrated further, but not concentrated to dryness. The resultant mixture was cooled down to at least 5±5° C. and agitated for at least 2 hours. The product was filtered, and the solids were washed with water (10.5 kg, 3 volumes). The filter cake was maintained under positive pressure of nitrogen for at least 30 minutes. The isolated product was dried to a constant weight under vacuum in a drying oven at a temperature of ≦70° C. with a nitrogen purge to yield 2-(2-(1H-pyrrolo[2,3-b]pyridin-3-yl)pyrimidin-4-ylamino)-2-methyl-N-(2,2,2-trifluoroethyl)butanamide (4.182 kg, white powder, 0.18% water content, 98.6% AUC using HPLC (method D)). Typical retention times are 4.4 minutes for (R)-2-(2-(1H-pyrrolo[2,3-b]pyridin-3-yl)pyrimidin-4-ylamino)-2-methylbutanoic acid and 6.2 minutes for 2-(2-(1H-pyrrolo[2,3-b]pyridin-3-yl)pyrimidin-4-ylamino)-2-methyl-N-(2,2,2-trifluoroethyl)butanamide.

Then general scheme, provided in Example 2 and the experimental description provided in Example 3 were used to generate the compounds in Table 1.

### Example 4

### JAK3 Inhibition Assay

Compounds were screened for their ability to inhibit JAK3 using the assay shown below. Reactions were carried out in a kinase buffer containing 100 mM HEPES (pH 7.4), 1 mM DTT, 10 mM MgCl₂, 25 mM NaCl, and 0.01% BSA. Substrate concentrations in the assay were 5 μM ATP (200 uCi/μmole ATP) and 1 μM poly(Glu)₄Tyr. Reactions were carried out at 25° C. and 1 nM JAK3.

To each well of a 96 well polycarbonate plate was added 1.5 μl of a compound of Formula I along with 50 μl of kinase buffer containing 2 μM poly(Glu)₄Tyr and 10 μM ATP. This was then mixed and 50 μl of kinase buffer containing 2 nM JAK3 enzyme was added to start the reaction. After 15 minutes at room temperature (25° C.), the reaction was stopped with 50 μl of 20% trichloroacetic acid (TCA) that also contained 0.4 mM ATP. The entire contents of each well were then transferred to a 96 well glass fiber filter plate using a TomTek Cell Harvester. After washing, 60 μl of scintillation fluid was added and ³³P incorporation detected on a Perkin Elmer TopCount.

### Example 5

### JAK2 Inhibition Assay

The assays were as described above in Example 4 except that JAK-2 enzyme, at a concentration of 5 nm, was used, the final poly(Glu)₄Tyr concentration was 15 μM, and final ATP concentration was 12 μM.

The data generated from these assays is provided in Table 2, below:

TABLE 2
Inhibition data for selected compounds of Formula I.

| Compound No. | JAK2 (μM) | JAK3 (μM) |
|---|---|---|
| 1 | 0.015 | 0.003 |
| 2 | 0.028 | 0.0032 |
| 3 | 0.028 | 0.0042 |
| 4 | 0.091 | 0.0056 |
| 5 | 0.0042 | 0.0024 |
| 6 | 0.046 | 0.0045 |
| 7 | 0.056 | 0.018 |
| 8 | 0.083 | 0.019 |
| 9 | 0.001 | 0.001 |
| 10 | 0.001 | 0.002 |
| 11 | 0.055 | 0.006 |
| 12 | 0.037 | 0.006 |
| 13 | 0.003 | 0.001 |
| 14 | 0.002 | 0.003 |
| 15 | 0.001 | 0.002 |
| 16 | 0.01 | 0.002 |
| 17 | 0.003 | 0.001 |
| 18 | 0.0007 | 0.001 |
| 19 | 0.001 | 0.004 |

### Example 6

### JAK3 Cellular Inhibition Assay

HT-2 clone A5E cells (ATCC Cat. # CRL-1841) were grown and maintained at 37° C. in a humidified incubator in cell culture medium (RPMI 1640 supplemented with 2 mM L-glutamine adjusted to contain 1.5 g/L sodium bicarbonate, 4.5 g/L glucose, 10 mM HEPES, 1.0 mM sodium pyruvate, 0.05 mM 2-mercaptoethanol, 10% fetal bovine serum, and 10% by volume rat T-STIM factor [Fisher Scientific Cat # CB40115] with Con A). On the day of the experiment, HT-2 cells were washed, resuspended at a density of 5×10⁶ cells per ml in fresh cell culture medium without T-STIM and incubated for 4 hours without T-STIM. After four hours, 50 μl (0.25×10⁶ cells) of the resuspended cells were added to each well of a 96 well plate. Serial dilutions of compounds were made in DMSO and then added to RPMI. 100 μl of the diluted compounds were added to each well and the plates were incubated for 1 hour at 37° C. 50 μl of recombinant murine interleukin-2 (rmIL-2) at 40 ng/ml (R & D systems Inc. Cat #402-ML) was added and the plates were incubated for 15 minutes at 37° C.

The plates were then centrifuged for 5 minutes at 1000 rpm, the supernatant was aspirated and 50 μl of 3.7% formaldehyde in phosphate buffered saline (PBS) was added per well. The plates were incubated for 5 minutes at room temperature on a plate shaker. The plates were again centrifuged at 1000 rpm for 5 minutes. The supernatant was aspirated, 50 μl of 90% methanol was added to each well, and the plate was incubated on ice for 30 minutes. The supernatant was aspirated and the plate washed with PBS. 25 μl per well of 1:10 diluted Phospho STAT-5 (Y694) PE conjugated antibody (PS-5 PE antibody; Becton-Dickinson Cat. #61256) was added to the plates and the plates were incubated for 45 minutes at room temperature on a plate shaker. 100 μl PBS was added and the plates were centrifuged. The supernatant was aspirated and the cells resuspended in 100 μl PBS. The plate was then read on a 96 well FACS reader (Guava PCA-96).

Compounds of the invention were found to inhibit JAK3 in this cellular assay.

### Example 7

### JAK2 Cellular Inhibition Assay

TF-1 cells (ATCC Cat. # CRL-2003) were grown and maintained at 37° C. in a humidified incubator in cell culture medium (RPMI 1640 supplemented with 2 mM L-glutamine adjusted to contain 1.5 g/L sodium bicarbonate, 4.5 g/L glucose, 10 mM HEPES, 1.0 mM sodium pyruvate, 10% fetal bovine serum and recombinant human granulocyte-macrophage colony stimulating factor [rhGMCSF, R&D Systems Inc. Cat. #215-GM]). On the day of the experiment, TF-1 cells were washed, resuspended at a density of 5×10⁶ cells per ml in fresh cell culture medium without rhGMCSF and incubated for 4 hours without rhGMCSF. After four hours, 50 μl (0.25×10⁶ cells) of the resuspended cells were added to each well of a 96 well plate. Serial dilutions of compounds were made in DMSO and then added to RPMI. 100 μl of the diluted compounds were added to each well and the plates were incubated for 1 hour at 37° C. 50 μl of rhGMCSF at 10 ng/m1 was added and the plates were incubated for 15 minutes at 37° C. The plates were then processed for FACS analysis as detailed above in Example 6.

Compounds of the invention were found to inhibit JAK2 in this cellular assay.

### Example 8

### Administration of Compound No. 9 to Mouse Model for Crohn's Disease and Ulcerative Colitis

Female SCID mice received grafts of 2.5×10e5 CD4+CD25-T cells from naïve BALB/c wild type donors (all Charles River Laboratories) to induce colitis. On day 14, after cell transfer, the recipient SCID mice were tested for graft acceptance and mice without T cells removed from the study. On days 19-33, the recipient SCID mice were administered compound no. 9 (methane sulfonic acid) mixed with vehicle (1% HPMC-c5, 50 mM Na citrate, pH 3.0) in doses of 50, 100, and 150 mpk b.i.d. Control mice were administered either saline, vehicle (1% HPMC-c5, 50 mM Na citrate, pH 3.0), or 1 mpk dexamethasone, b.i.d. Doses were administered via oral gavage. On day 35, the mice were sacrificed.

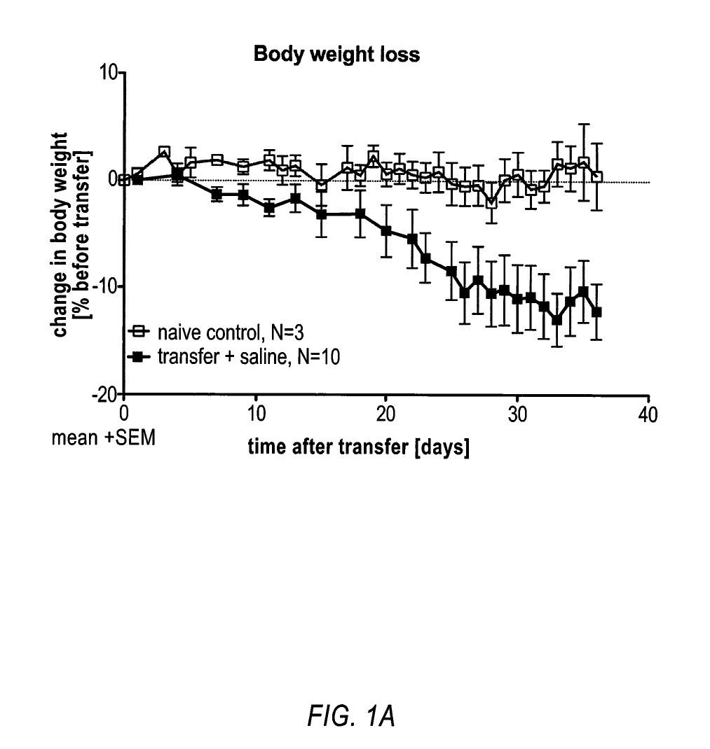

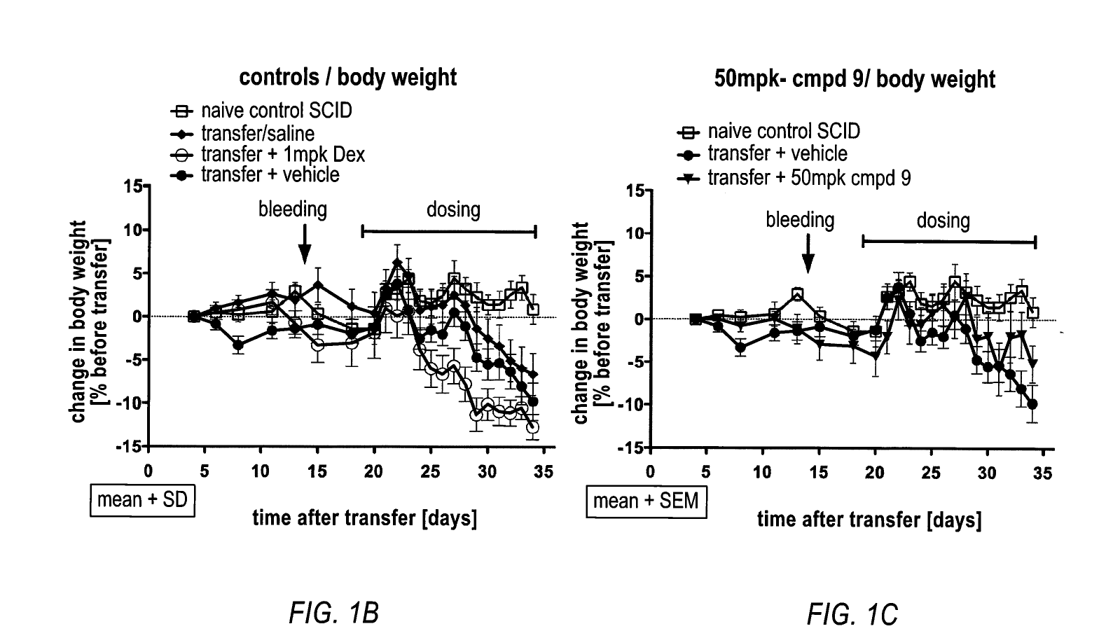

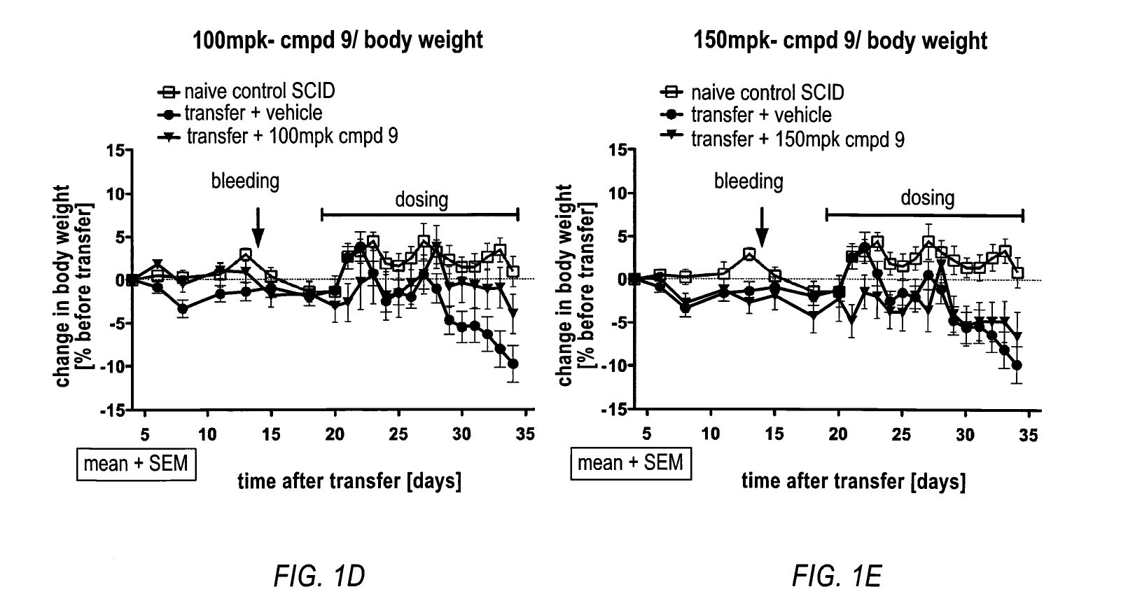

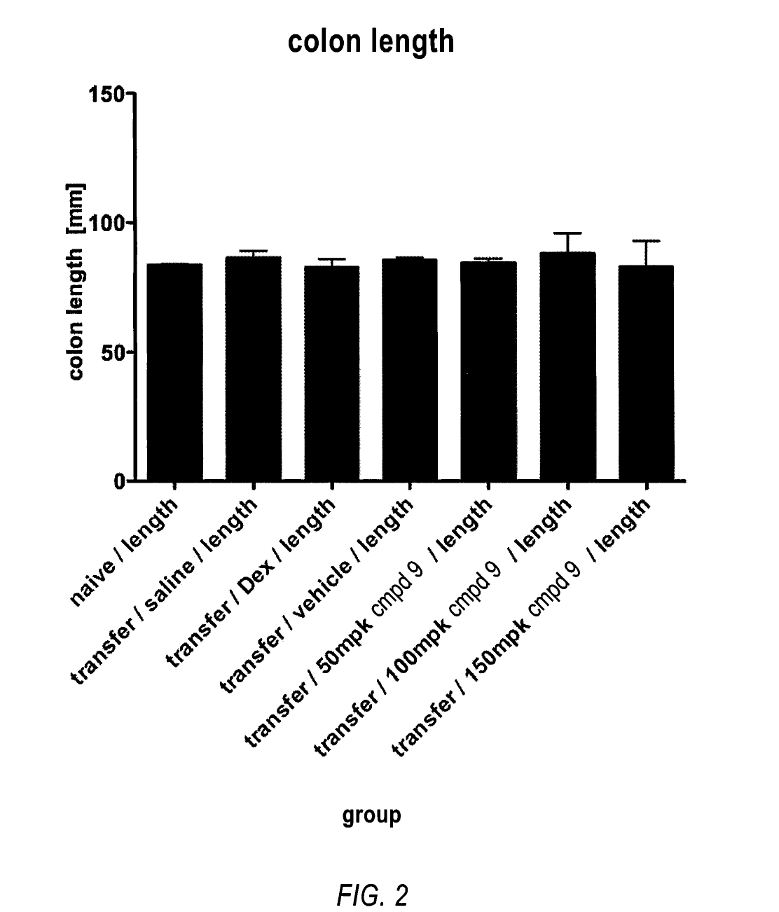

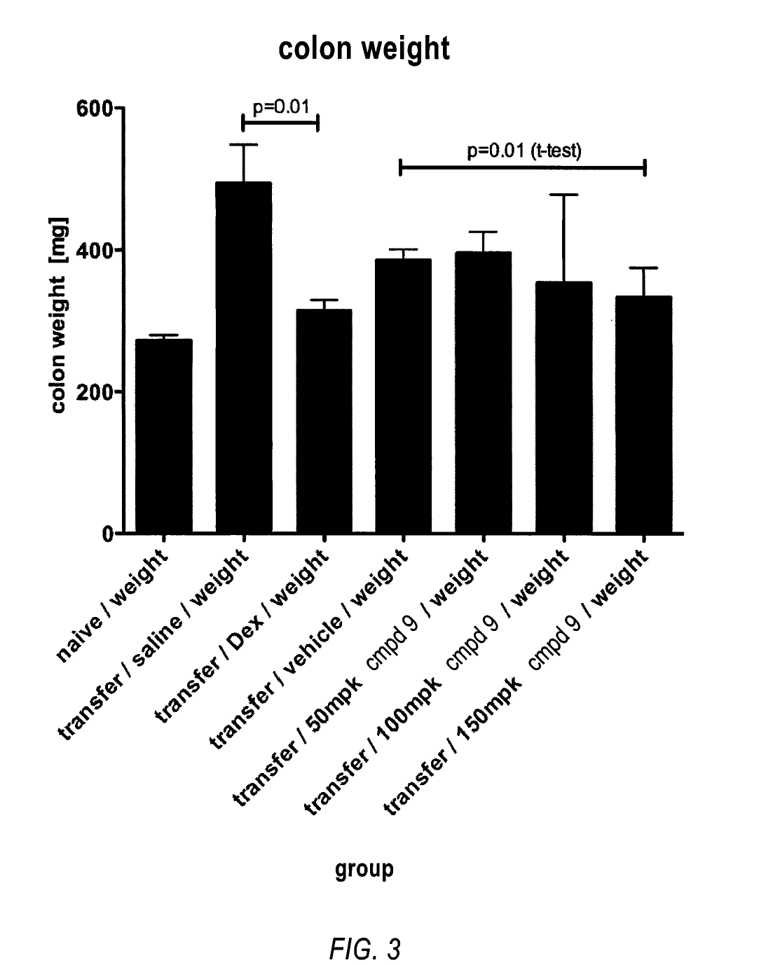

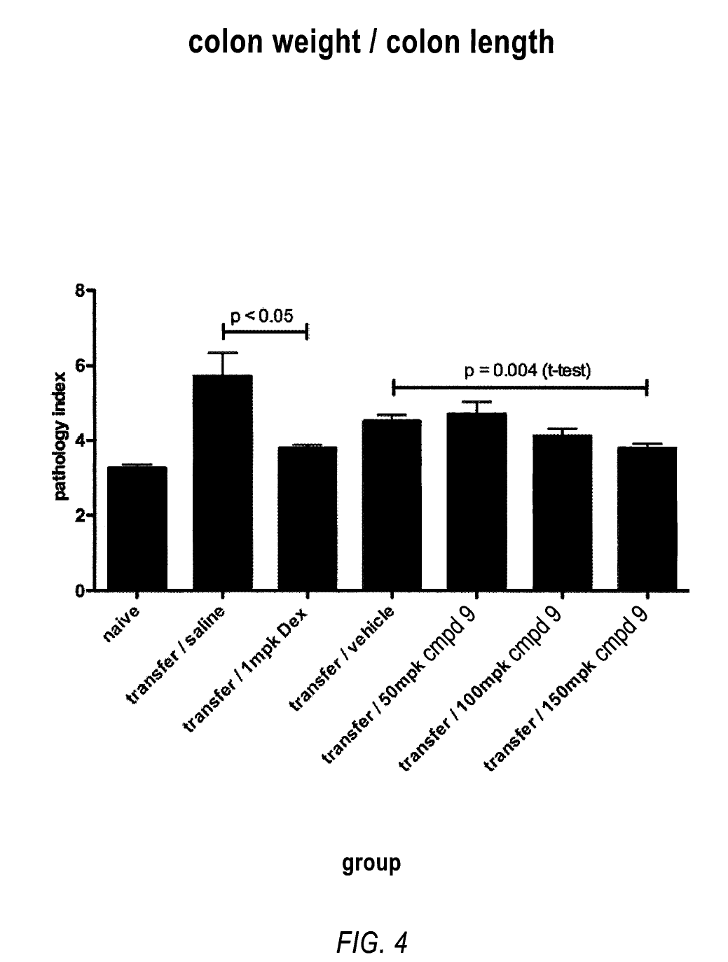

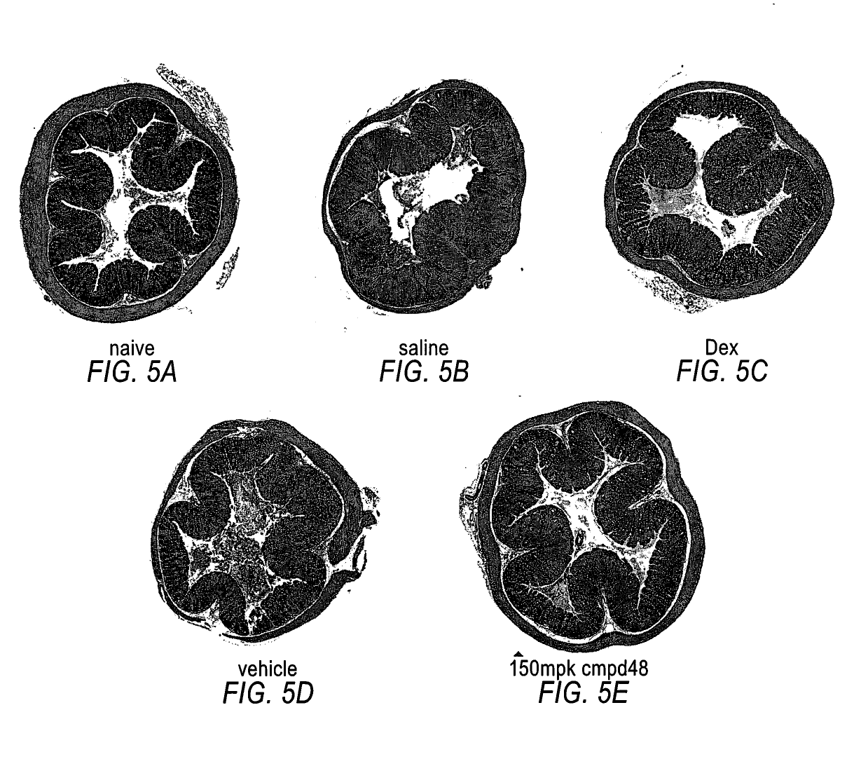

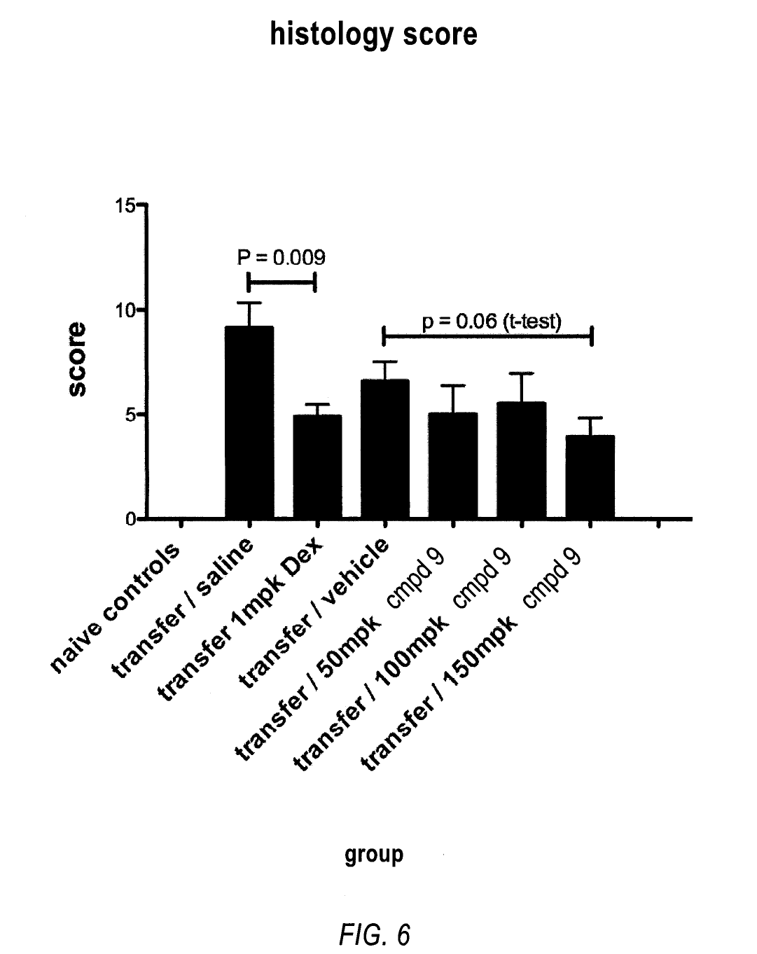

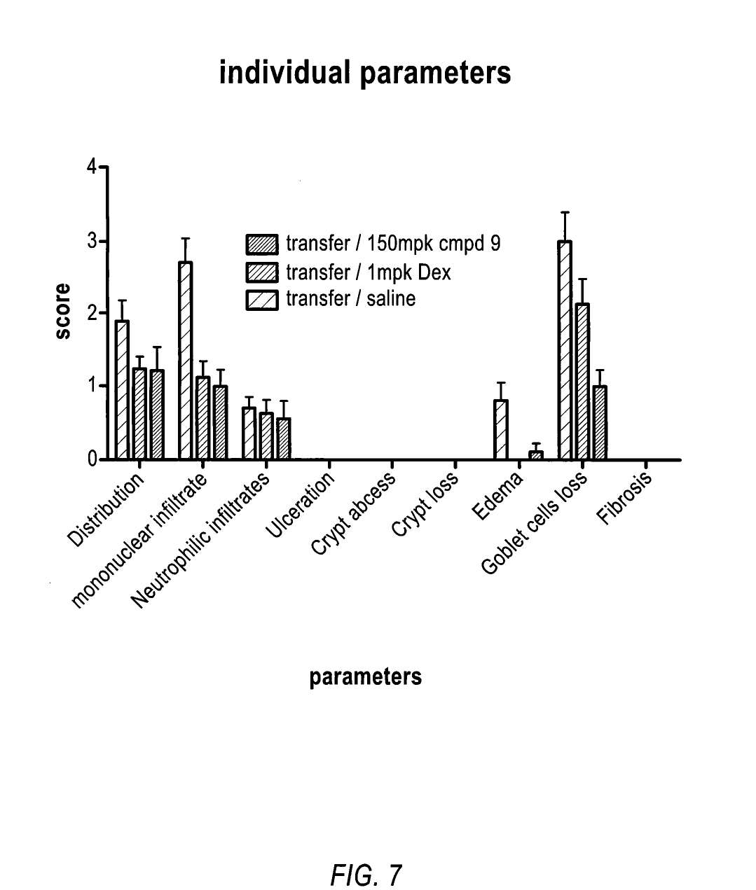

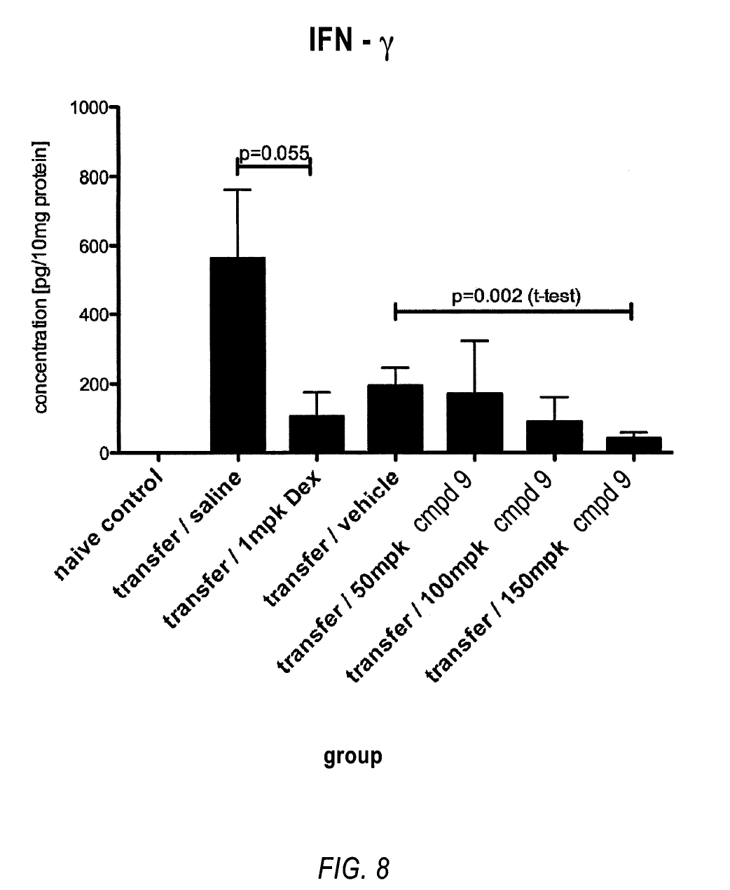

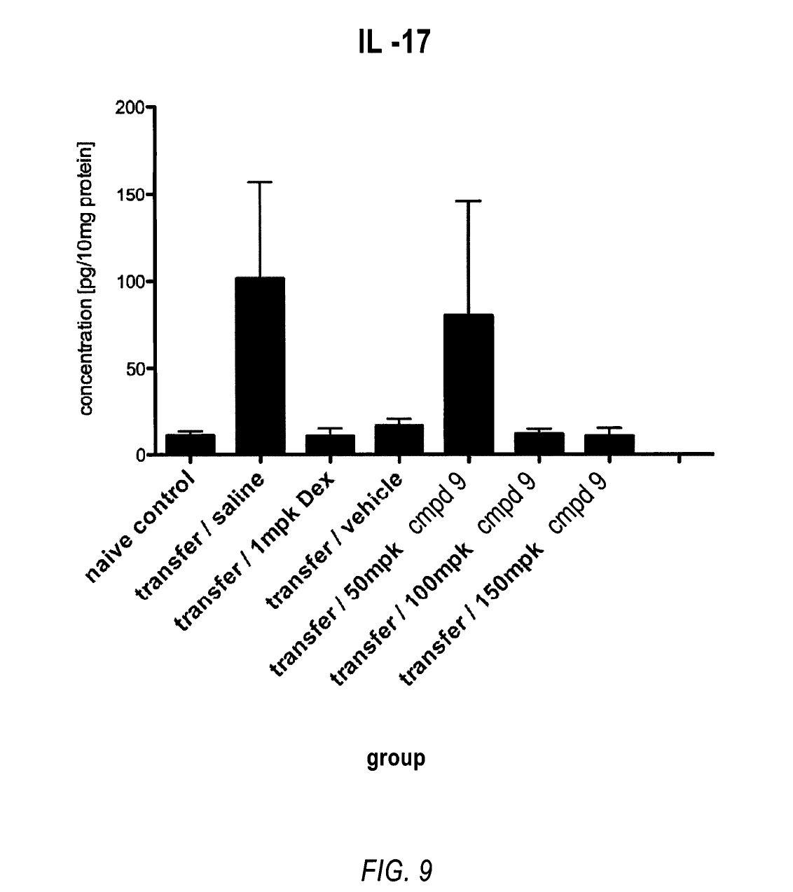

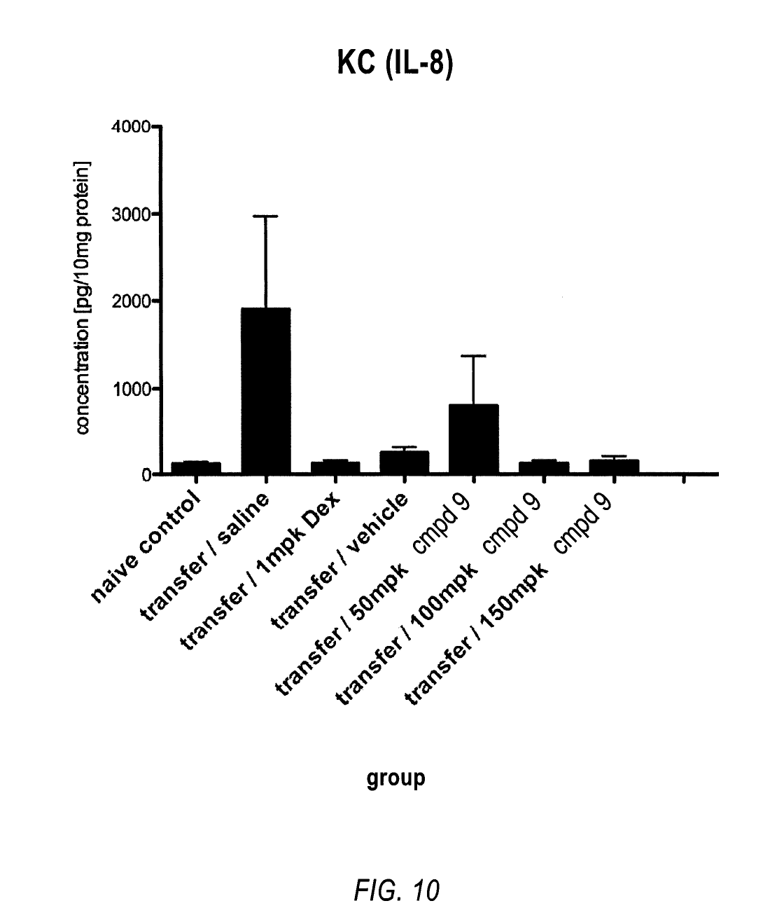

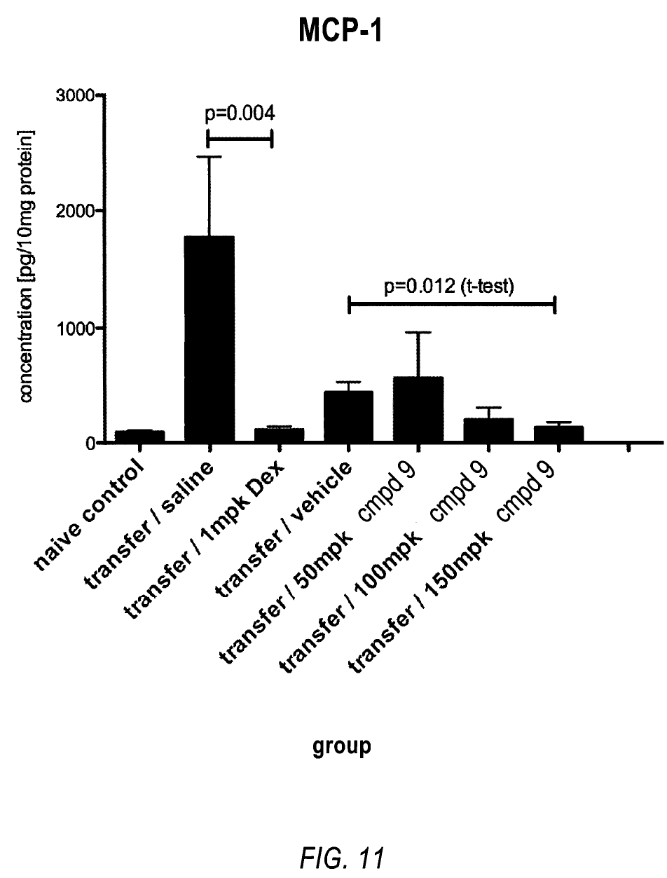

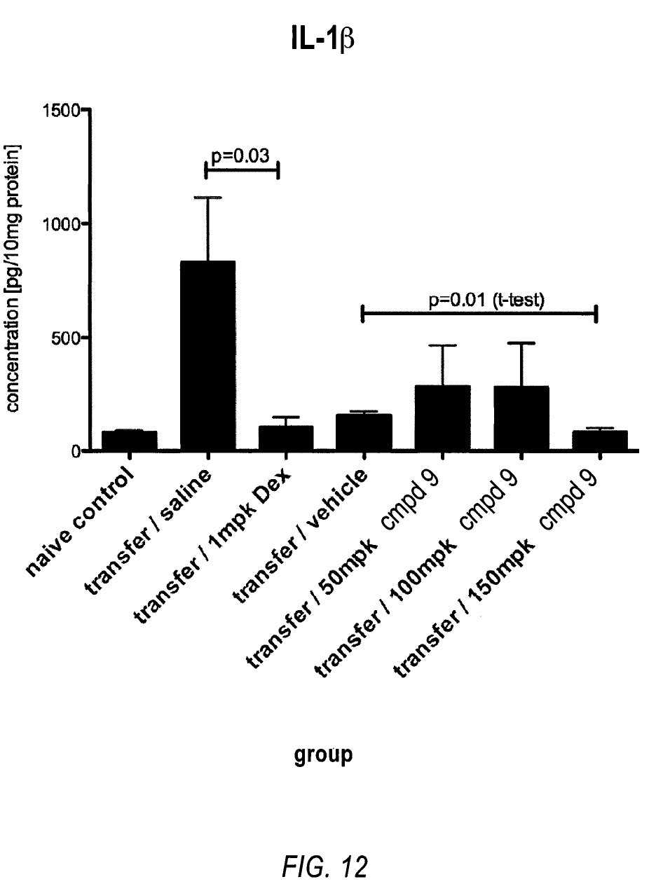

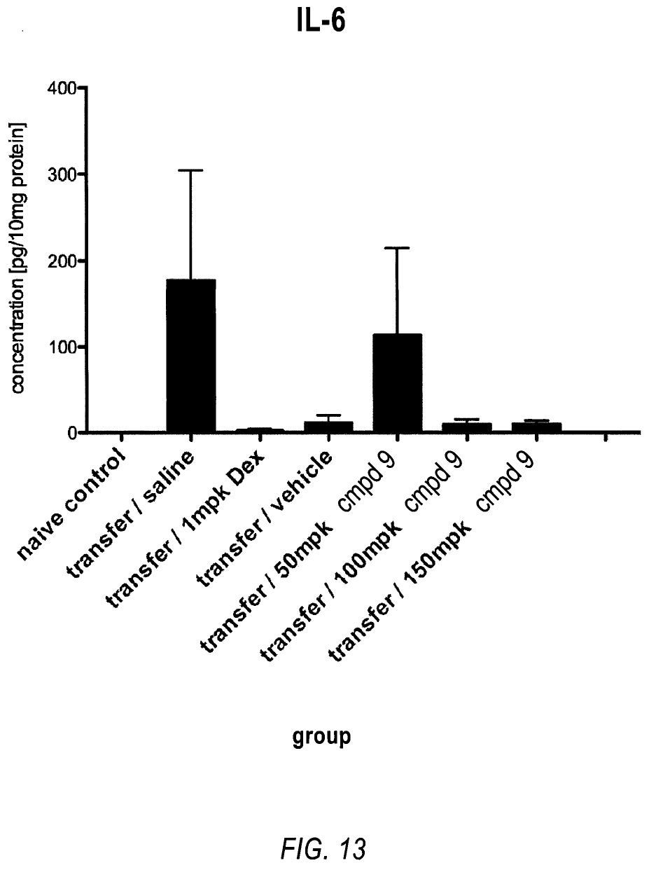

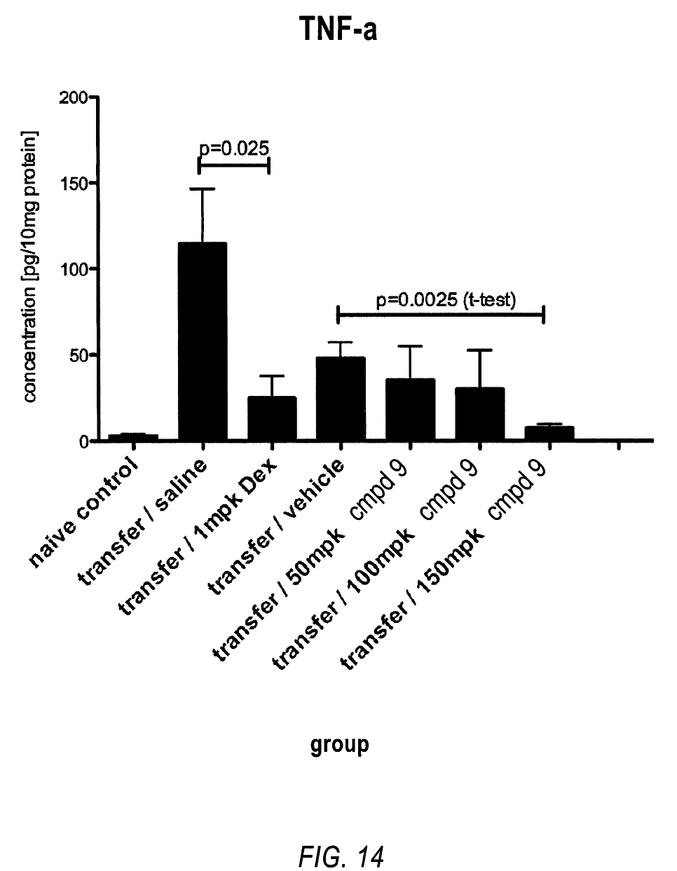

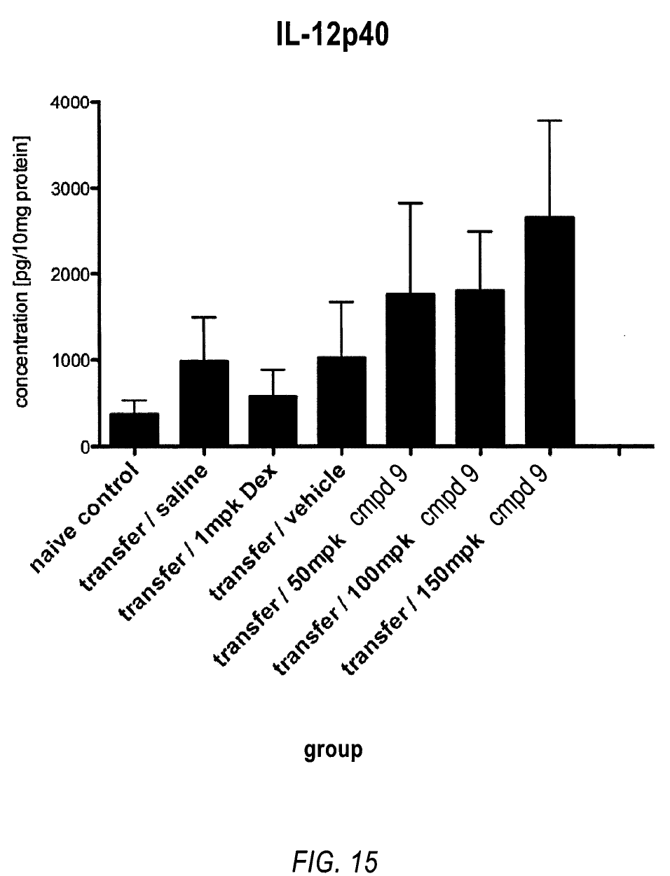

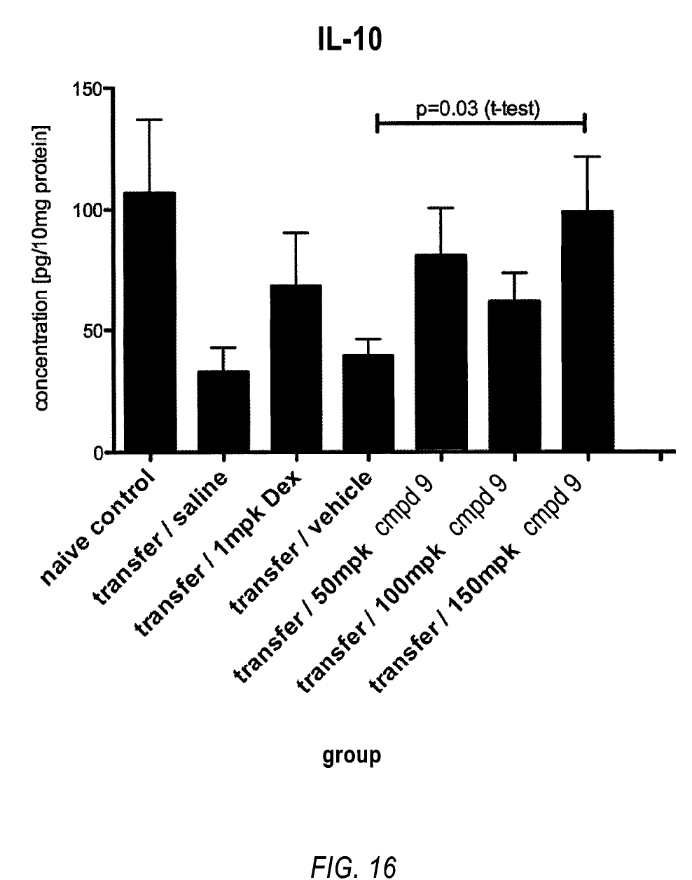

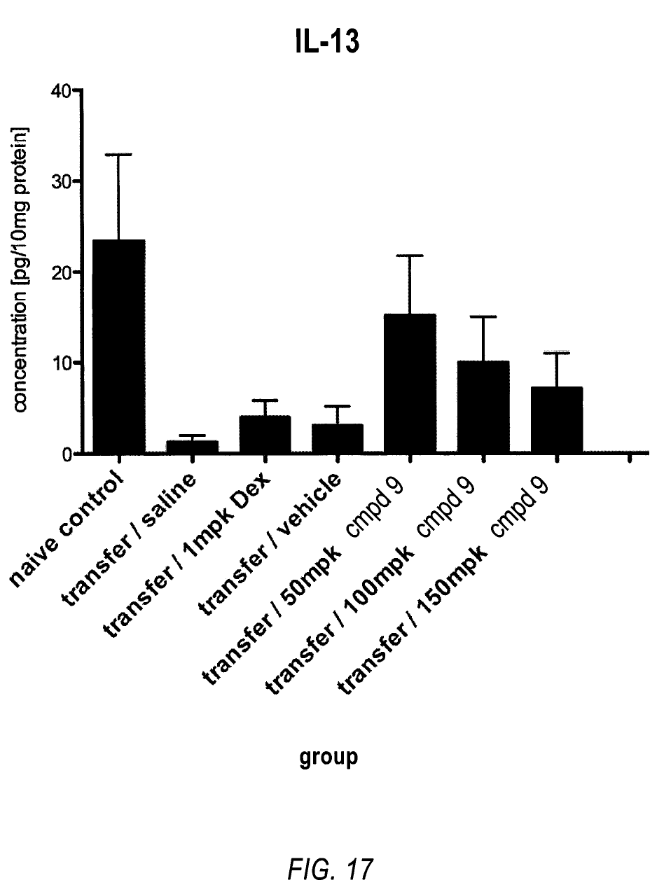

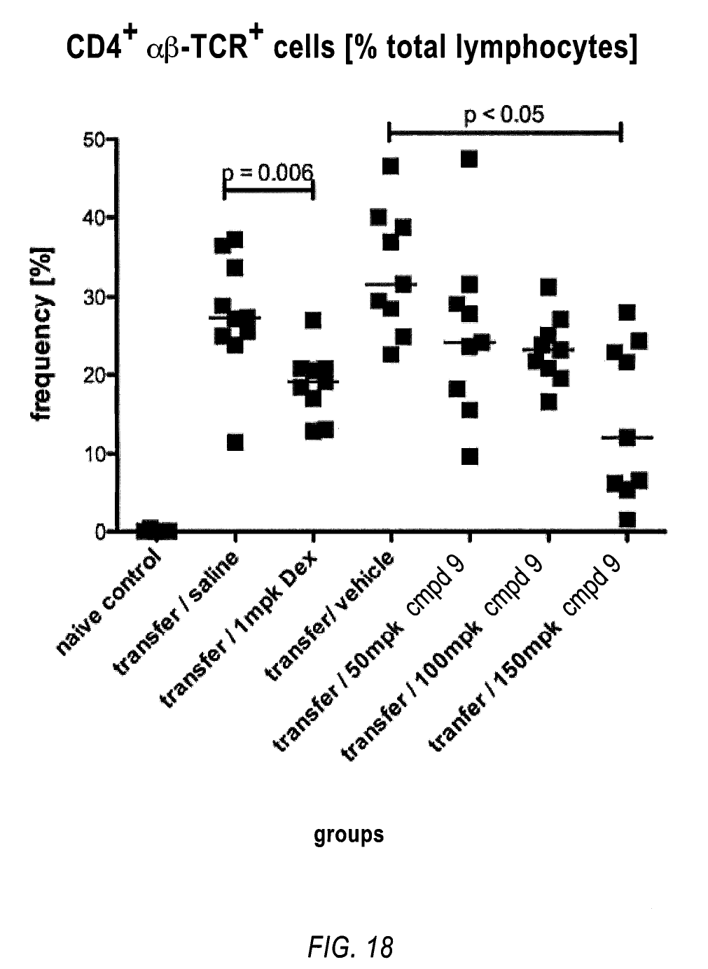

The data from this experiment, illustrated in FIGS. 1A-18, demonstrate that the therapeutic administration of compound no. 9 in the mouse colitis model improved colon pathology and inflammatory cytokine production. Improvements were also observed in colon histology and a significant reduction in frequency of splenic CD4+ T cells. Thus, the use of JAK (i.e., JAK2 and JAK3) as the therapeutic target for treating ulcerative colitis and Crohn's disease, is verified.

### Example 9

### Manufacture of Tablets

A formulation is provided in Table 3 for Exemplary Tablet 1 comprising 50 mg of API, i.e., a compound of Formula I.

TABLE 3
Exemplary Tablet 1

| | Amt. Per | |
|---|---|---|
| Ingredient | Tablet (mg) | wt % |
| Compound of Formula I | 50.00 | 20 |
| Binder (Avicel PH 102) | 92.8125 | 37.125 |
| Diluent (Lactose Monohydrate, NF (Fast-Flo 316) | 92.8125 | 37.125 |
| Disintegrant (AcDiSol) | 7.5 | 3 |
| Glidant (CaBoSil) | 2.5 | 1 |
| Wetting Agent (Sodium Lauryl Sulfate, NF) | 2.5 | 1 |
| Lubricant (Magnesium Stearate) | 1.875 | 0.75 |
| Total | 250 | 100 |

A formulation is provided in Table 4 for Exemplary Tablet 2 comprising 25 mg of API, i.e., a compound of Formula I.

TABLE 4
Exemplary Tablet 2.

| | Amt. Per | |
|---|---|---|
| Ingredient | Tablet (mg) | wt % |
| Compound of Formula I | 25.00 | 10 |
| Binder (Avicel PH 102) | 105.31 | 42.125 |
| Diluent (Lactose Monohydrate, NF (Fast-Flo 316) | 105.31 | 42.125 |
| Disintegrant (AcDiSol) | 7.5 | 3 |
| Glidant (CaBoSil) | 2.5 | 1 |
| Wetting Agent (Sodium Lauryl Sulfate, NF) | 2.5 | 1 |
| Lubricant (Magnesium Stearate) | 1.875 | 0.75 |
| Total | 250 | 100 |

### Example 10

### Exemplary Tablet 1 (Formulated to have 50 Mg of a Compound of Formula I)

A batch of 250 mg total weight tablets can be formulated to have approximately 50 mg of compound of Formula I per tablet using the amounts of ingredients recited in Table 3, above.

Sieve the compound of Formula I, microcrystalline cellulose (FMC MCC Avicel® PH102, commercially available from FMC BioPolymer Corporation of Philadelphia, Pa.), lactose (Foremost FastFlo® Lactose #316 commercially available from Foremost Farms USA of Baraboo, Wis.), sodium croscarmellose (FMC Ac-Di-Sol®, commercially available from FMC BioPolymer Corporation of Philadelphia, Pa.), sodium lauryl sulfate (commercially available from Stepan Company of Northfield, Ill.), and colloidal silicon dioxide (Cabot Cab-O-Sil® M-5P Fumed Silicon Dioxide, commercially available from Cabot Corporation of Alpharetta, Ga.) through a 20 mesh screen to remove lumps.

Add these sieved ingredients to a 16 quart V-blender and blend for about 20 minutes in a V-blender at 10-12 rpm.

Sieve magnesium stearate (commercially available from Mallinckrodt, Inc.) through a 20 mesh screen to remove lumps, and add to the blended mixture. Blend the second mixture containing the newly added magnesium stearate for another 4 minutes at a speed of about 10 to 24 rpm.

Once the final blend has been completed, transfer the mixture to a tablet press (e.g., a Piccola B-Tooling, 10 Station rotary tablet press (half tooled)) for compression into 250 mg tables having approximately 50 mg of a compound of Formula I.

### Example 11

### Exemplary Tablet 2 (Formulated to have 25 Mg of a Compound of Formula I)

A batch of 250 mg total weight tablets can be formulated to have approximately 25 mg of a compound of Formula I per tablet using the amounts of ingredients recited in Table B, above.

Sieve the compound of Formula I, microcrystalline cellulose (FMC MCC Avicel®PH102, commercially available from FMC BioPolymer Corporation of Philadelphia, Pa.), lactose (Foremost FastFlo® Lactose #316 commercially available from Foremost Farms USA of Baraboo, Wis.), sodium croscarmellose (FMC Ac-Di-Sol®, commercially available from FMC BioPolymer Corporation of Philadelphia, Pa.), sodium lauryl sulfate (commercially available from Stepan Company of Northfield, Ill.), and colloidal silicon dioxide (Cabot Cab-O-Sil® M-5P Fumed Silicon Dioxide, commercially available from Cabot Corporation of Alpharetta, Ga.) through a 20 mesh screen to remove lumps.

Add these sieved ingredients to a 16 quart V-blender and blend for about 20 minutes in a V-blender at 10-12 rpm.

Sieve magnesium stearate (commercially available from Mallinckrodt, Inc.) through a 20 mesh screen to remove lumps, and add to the blended mixture. Blend the second mixture containing the newly added magnesium stearate for another 4 minutes at a speed of about 10 to 24 rpm.

Once the final blend has been completed, transfer the mixture to a tablet press (e.g., a Piccola B-Tooling, 10 Station rotary tablet press (half tooled)) for compression into 250 mg tables having approximately 25 mg of the compound of Formula I.

### Example 12

### Exemplary Capsule (Formulated to have 25 Mg of the Compound of Formula I)

Add 25 mg of a compound of Formula I to one half of a 2-piece gelatin capsule, and cap the filled half of the gelatin capsule with the second half of the 2-piece capsule.

### Example 13

### Exemplary Capsule (Formulated to have 50 mg of a Compound of Formula I)

Add 50 mg of a compound of Formula I to one half of a 2-piece gelatin capsule, and cap the filled half of the gelatin capsule with the second half of the 2 piece capsule.

### Example 14

### Exemplary Capsule (Formulated to have 75 Mg of a Compound of Formula I)

Add 75 mg of the compound of Formula I to one half of a 2-piece gelatin capsule, and cap the filled half of the gelatin capsule with the second half of the 2 piece capsule.

### Example 15

### Administration of Pharmaceutical Formulations

### Example 15A

### Exemplary Administration A

Human patients are orally administered a pharmaceutical formulation according to Table 5:

TABLE 5
Exemplary administration of pharmaceutical formulations of
the present invention.

| Frequency of | |
|---|---|
| dosing (per day) | Dosage |
| 2 | Tablet 2 |
| 2 | Tablet 1 or 2 × Tablet 2 |
| 2 | 2 × Tablet 1 or 4 × Tablet 2 |
| 2 | 3 × Tablet 1 or 6 × Tablet 2 |

The pharmaceutical formulations may be administered to subjects in the morning, e.g., between 6:00 AM and 12:00 PM, and evening, e.g., between 5:00 PM and 11:00 PM, and in some administrations, the pharmaceutical formulation is given at approximately the same time (within a 1-hour window) on each dosing occasion.

Furthermore, the tablets (e.g., Tablet 1 and/or Tablet 2) may be administered with or without a fluid (e.g., water or other beverage). Also, human patients being administered the tablet(s) may fast for a period of time prior to or after the administration.

In several instances, the administration of the tablet(s) may last for a period of about 12 weeks.

### Example 15B

### Exemplary Administration B

Human patients are orally administered a pharmaceutical formulation according to Table 6:

TABLE 6
Exemplary administration of pharmaceutical formulations of
the present invention.

| Frequency of | |
|---|---|
| dosing | Dosage |
| 12 hr. intervals | Tablet 2 |
| 12 hr. intervals | Tablet 1 or 2 × Tablet 2 |
| 12 hr. intervals | 2 × Tablet 1 or 4 × Tablet 2 |
| 12 hr. intervals | 3 × Tablet 1 or 6 × Tablet 2 |

The pharmaceutical formulations may be administered to subjects in the morning, e.g., between 5:00 AM and 12:00 PM, and evening, e.g., between 5:00 PM and 12:00 AM, and in some administrations, the pharmaceutical formulation is given at approximately the same time (within a 1-hour window) on each dosing occasion.

Furthermore, the tablet(s) (e.g., Tablet 1 and/or Tablet 2) may be administered with or without a fluid (e.g., water or other beverage). Also, human patients being administered the tablet may fast for a period of time prior to or after the administration.

In several instances, the administration of the tablet(s) lasts for a period of about 12 weeks.

### Example 16

### Administration of Pharmaceutical Formulations

### Example 16A

### Exemplary Administration A

Human patients are orally administered a pharmaceutical formulation according to Table 7:

TABLE 7
Exemplary administration of pharmaceutical formulations of
the present invention.

| Frequency of | |
|---|---|
| dosing (per day) | Dosage |
| 2 | Tablet 2 |
| 1 | Tablet 1 or 2 × Tablet 2 |
| 1 | 2 × Tablet 1 or 4 × Tablet 2 |
| 1 | 3 × Tablet 1 or 6 × Tablet 2 |

When frequency of dosing is twice a day, the pharmaceutical formulations may be administered to subjects in the morning, e.g., between 6:00 AM and 12:00 PM, and evening, e.g., between 5:00 PM and 11:00 PM, and in some administrations, the pharmaceutical formulation is given at approximately the same time (within a 1-hour window) on each dosing occasion. In instances where the frequency dosing is once per day, the pharmaceutical formulation may be given anytime during the day, and in some administrations, the pharmaceutical formulation is given at approximately the same time (within a 1-hour window) on each dosing occasion.

Furthermore, the tablets (e.g., Tablet 1 and/or Tablet 2) may be administered with or without a fluid (e.g., water or other beverage). Also, human patients being administered the tablet(s) may fast for a period of time prior to or after the administration.

In several instances, the administration of the tablet(s) may last for a period of about 12 weeks.

### Example 16B

### Exemplary Administration B

Human patients are orally administered a pharmaceutical formulation according to Table 8:

TABLE 8
Exemplary administration of pharmaceutical formulations of
the present invention.

| Frequency of | |
|---|---|
| dosing | Dosage |
| 24 hr. intervals | Tablet 2 |
| 24 hr. intervals | Tablet 1 or 2 × Tablet 2 |
| 24 hr. intervals | 2 × Tablet 1 or 4 × Tablet 2 |
| 24 hr. intervals | 3 × Tablet 1 or 6 × Tablet 2 |

The pharmaceutical formulations may be administered to subjects anytime during the 24 hr. interval, and in some administrations, the pharmaceutical formulation is given at approximately the same time (within a 1-hour window) on each dosing occasion.

Furthermore, the tablet(s) (e.g., Tablet 1 and/or Tablet 2) may be administered with or without a fluid (e.g., water or other beverage). Also, human patients being administered the tablet may fast for a period of time prior to or after the administration.

In several instances, the administration of the tablet(s) lasts for a period of about 12 weeks.

### Example 17

### Administration of Pharmaceutical Formulations

### Example 17A

### Exemplary Administration A

Human patients are orally administered a pharmaceutical formulation according to Table 9:

TABLE 9
Exemplary administration of pharmaceutical formulations of
the present invention.

| Frequency of | |
|---|---|
| dosing (per day) | Dosage |
| q.d. | 100 mg of API (e.g., 2 × Tablet 1) |
| q.d. | 150 mg of API (e.g., 3 × Tablet 1) |
| q.d. | 200 mg of API (e.g., 4 × Tablet 1) |
| b.i.d. | 100 mg of API (e.g., 2 × Tablet 1) |

The pharmaceutical formulations may be administered to subjects in the morning, e.g., between 6:00 AM and 12:00 PM, and evening, e.g., between 5:00 PM and 11:00 PM, and in some administrations, the pharmaceutical formulation is given at approximately the same time (within a 1-hour window) on each dosing occasion.

Furthermore, the tablets (e.g., Tablet 1) may be administered with or without a fluid (e.g., water or other beverage). Also, human patients being administered the tablet(s) may fast for a period of time prior to or after the administration.

In several instances, the administration of the tablet(s) may last for a period of about 12 weeks.

### OTHER EMBODIMENTS

All publications and patents referred to in this disclosure are incorporated herein by reference to the same extent as if each individual publication or patent application were specifically and individually indicated to be incorporated by reference. Should the meaning of the terms in any of the patents or publications incorporated by reference conflict with the meaning of the terms used in this disclosure, the meaning of the terms in this disclosure are intended to be controlling. Furthermore, the foregoing discussion discloses and describes merely exemplary embodiments of the present invention. One skilled in the art will readily recognize from such discussion and from the accompanying drawings and claims, that various changes, modifications and variations can be made therein without departing from the spirit and scope of the invention as defined in the following claims.

What is claimed is:

1. A method for treating or lessening the severity of a disease selected from spondyloarthropathy, systemic lupus erythematosus, rheumatoid arthritis, or any combination thereof comprising administering to a patient in need thereof a chemotherapy agent and a compound of Formula I 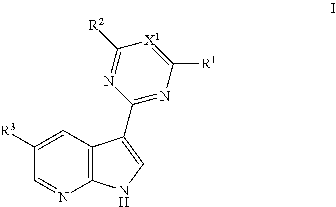
or a pharmaceutically acceptable salt thereof, wherein:
X¹ is N or CR⁴;
R² is H or halo;
R³ is H or halo;
R⁴ is H or halo;
R¹ is 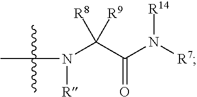
R″ is H or an unsubstituted C₁₋₂ aliphatic;
R⁸ is an unsubstituted C₁₋₄ aliphatic;
R⁹ is an unsubstituted C₁₋₄ aliphatic;
R⁷ is a C₁₋₃ aliphatic optionally substituted with up to 3 occurrences of F; and
R¹⁴ is H or unsubstituted C₁₋₂ alkyl.

2. The method of claim 1, wherein the chemotherapy agent comprises methotrexate, azathioprine, cyclosporine, cyclophosphamide, 6-mercaptopurine, or any combination thereof.

3. The method of either of claim 1 or 2, wherein the chemotherapy agent comprises an injectable formulation or an oral formulation.

4. The method of any one of claims 1-3, wherein the patient is administered from about 5 mg to about 100 mg of the chemotherapy agent per month.

5. The method of any one of claims 1-4, wherein R² is H or F.

6. The method of any one of claims 1-5, wherein R³ is H or Cl.

7. The method of any one of claims 1-6, wherein each of R⁸ and R⁹ is independently selected from methyl, ethyl, propyl, iso-propyl, butyl, or tert-butyl.

8. The method of any one of claims 1-7, wherein each of R⁸ and R⁹ is independently selected from methyl or ethyl.

9. The method of any one of claims 1-8, wherein R¹⁴ is H or methyl.

10. The method of any one of claims 1-9, wherein R⁷ is an unsubstituted C₁₋₃ aliphatic.

11. The method of any one of claims 1-9, wherein R⁷ is a C₁₋₃ aliphatic substituted with 1-3 occurrences of F.

12. The method of any one of claims 1-9, wherein R⁷ is a group selected from —CH₂CH₃, —CH₂CF₃, —CH₂CH₂CH₃, or —CHCH₃CH₃.

13. The method of claim 1, wherein the compound of Formula I is selected from:

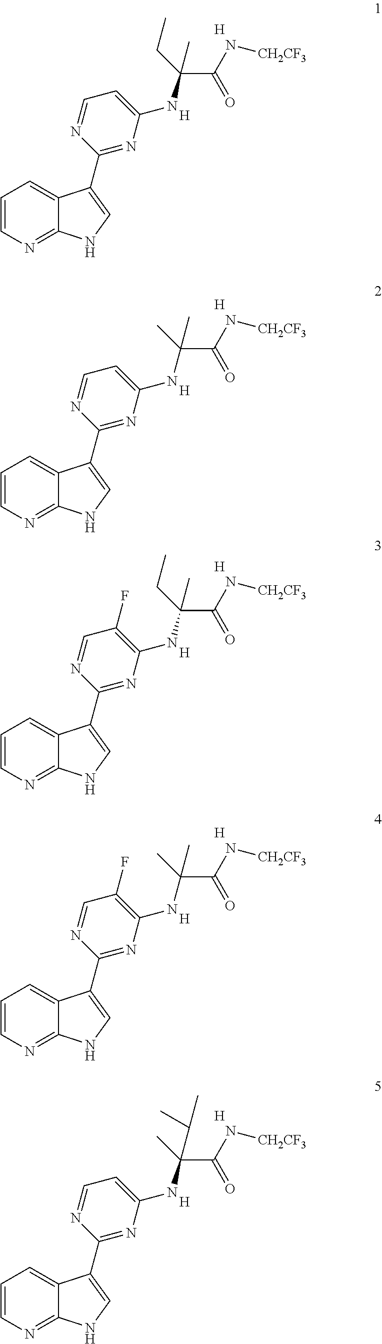

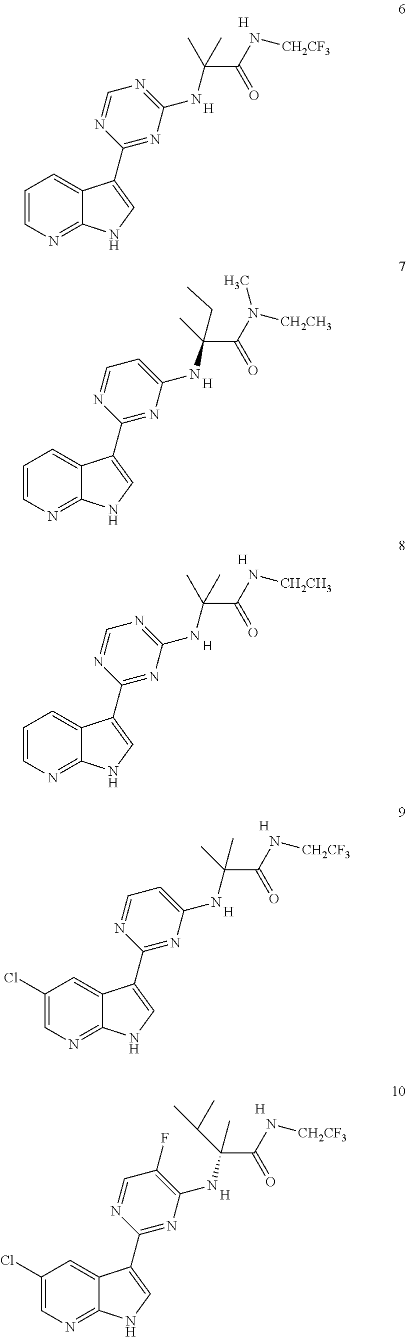

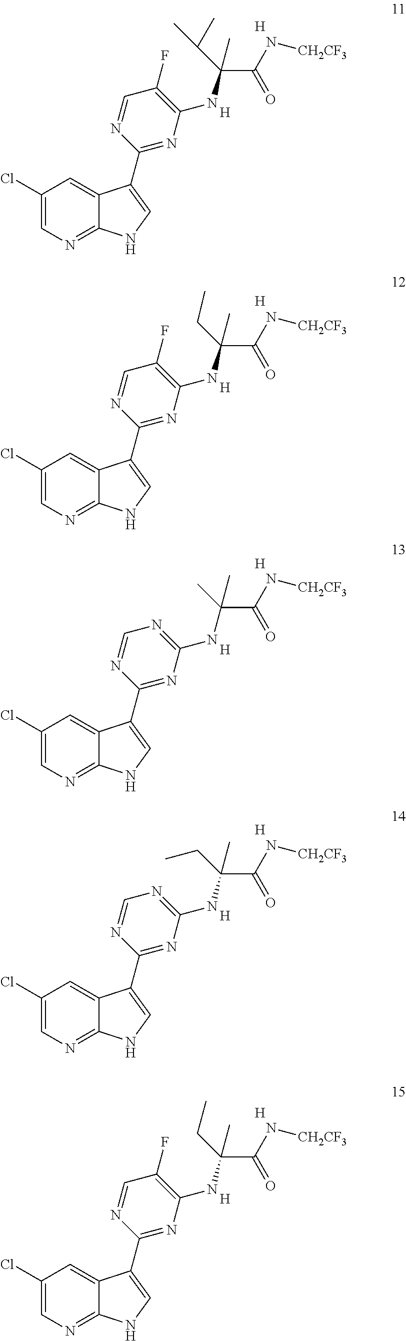

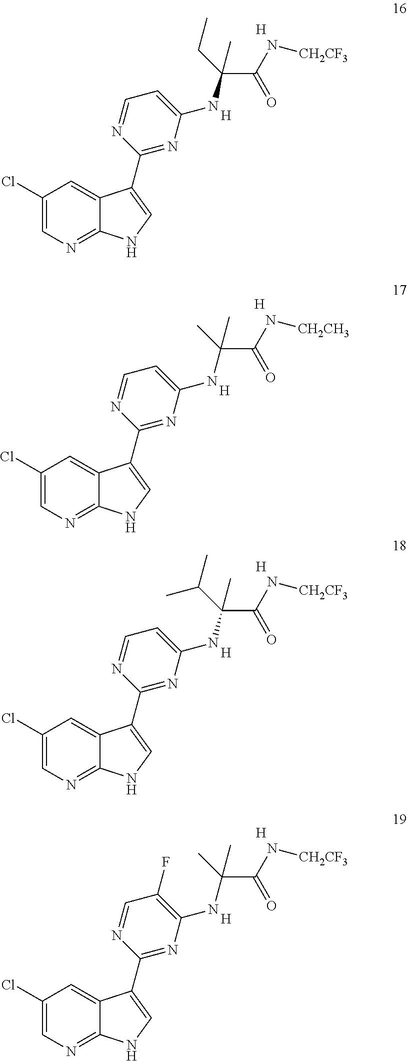

14. The method of any one of claims 1-13, wherein the compound of Formula I is administered at least once per day.

15. The method of any one of claims 1-14, wherein the compound of Formula I is administered at least twice per day.

16. The method of any one of claims 1-15, wherein the compound of Formula I is orally administered to the patient in need thereof.

17. The method of any one of claims 1-14, wherein at least about 100 mg of the compound of Formula I is administered to the patient once per day.

18. The method of any one of claims 1-14, wherein at least about 150 mg of the compound of Formula I is administered to the patient once per day.

19. The method of any one of claims 1-14, wherein at least about 200 mg of the compound of Formula I is administered to the patient once per day.

20. The method of any one of claims 1-16, wherein at least about 100 mg of the compound of Formula I is administered to the patient twice per day.

21. The method of any one of claims 1-20, wherein the spondyloarthropathy is a condition selected from peripheral spondyloarthropathy, axial spondyloarthropathy, reactive arthritis, Reiter's syndrome, psoriatic arthritis, ankylosing spondylitis, ulcerative colitis, Crohn's disease, or any combination thereof.

22. A method for treating or lessening the severity of a disease selected from systemic lupus erythematosus, ulcerative colitis, Crohn's disease, ankylosing spondylitis, reactive arthritis, Reiter's syndrome, psoriatic arthritis, peripheral spondyloarthropathy, axial spondyloarthropathy, or any combination thereof comprising administering to a patient in need thereof a compound of Formula I 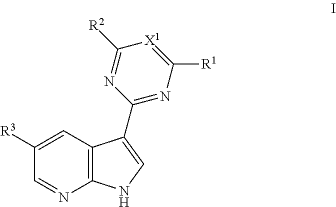
or a pharmaceutically acceptable salt thereof, wherein:
X¹ is N or CR⁴;
R² is H or halo;
R³ is H or halo;
R⁴ is H or halo;
R¹ is 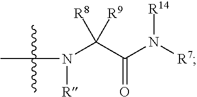
R″ is H or an unsubstituted C₁₋₂ aliphatic;
R⁸ is an unsubstituted C₁₋₄ aliphatic;
R⁹ is an unsubstituted C₁₋₄ aliphatic;
R⁷ is a C₁₋₃ aliphatic optionally substituted with up to 3 occurrences of F; and
R¹⁴ is H or unsubstituted C₁₋₂ alkyl.

23. The method of claim 22, further comprising administering a chemotherapy agent to the patient.

24. The method of claim 22, wherein the chemotherapy agent comprises methotrexate, azathioprine, cyclosporine, cyclophosphamide, 6-mercaptopurine, or any combination thereof.

25. The method of either of claim 23 or 24, wherein the chemotherapy agent comprises an injectable formulation or an oral formulation.

26. The method of any one of claims 23-25, wherein the patient is administered from about 5 mg to about 100 mg of the chemotherapy agent per month.

27. The method of any one of claims 22-26, wherein R² is H or F.

28. The method of any one of claims 22-27, wherein R³ is H or Cl.

29. The method of any one of claims 22-28, wherein each of R⁸ and R⁹ is independently selected from methyl, ethyl, propyl, iso-propyl, butyl, or tert-butyl.

30. The method of any one of claims 22-29, wherein each of R⁸ and R⁹ is independently selected from methyl or ethyl.

31. The method of any one of claims 22-30, wherein R¹⁴ is H or methyl.

32. The method of any one of claims 22-31, wherein R⁷ is an unsubstituted C₁₋₃ aliphatic.

33. The method of any one of claims 22-31, wherein R⁷ is a C₁₋₃ aliphatic substituted with 1-3 occurrences of F.

34. The method of any one of claims 22-31, wherein R⁷ is a group selected from —CH₂CH₃, —CH₂CF₃, —CH₂CH₂CH₃, or —CHCH₃CH₃.

35. The method of claim 22, wherein the compound of Formula I is selected from:

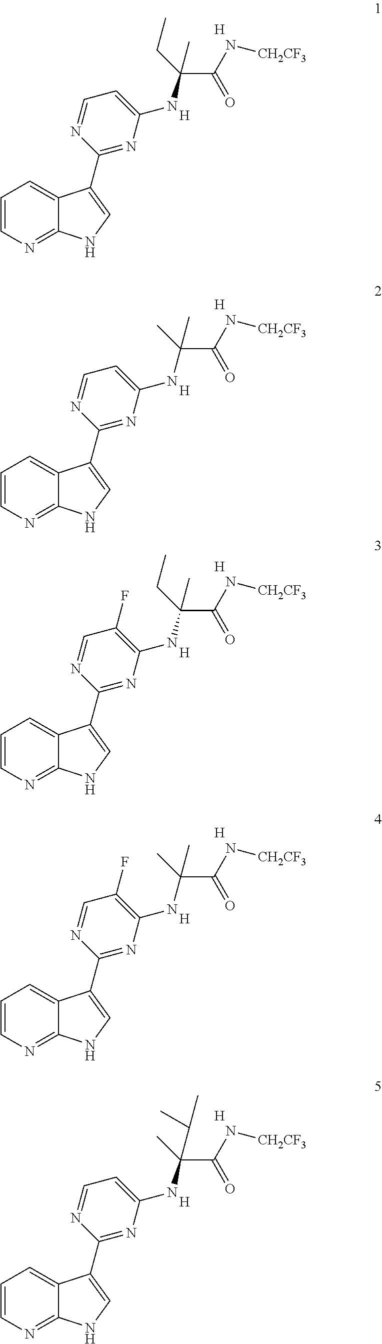

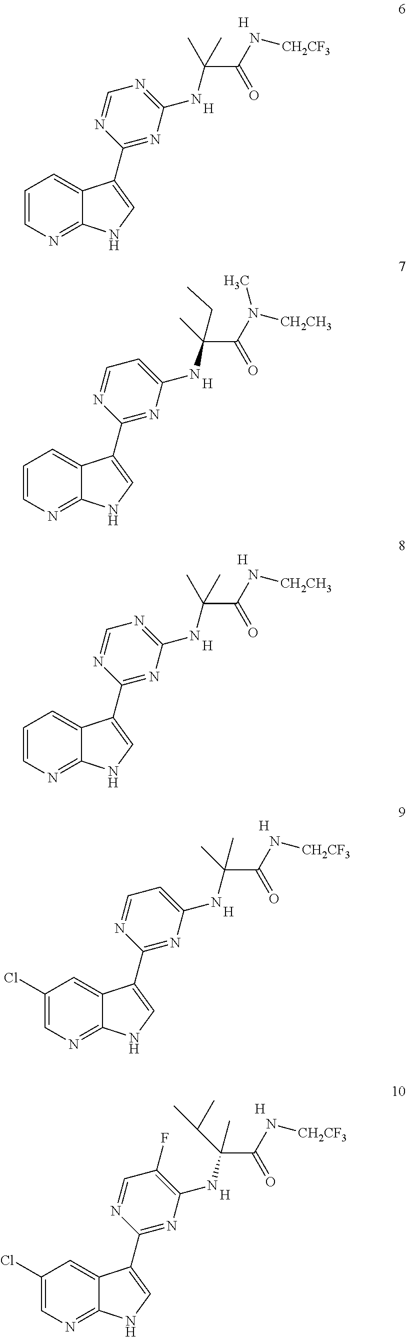

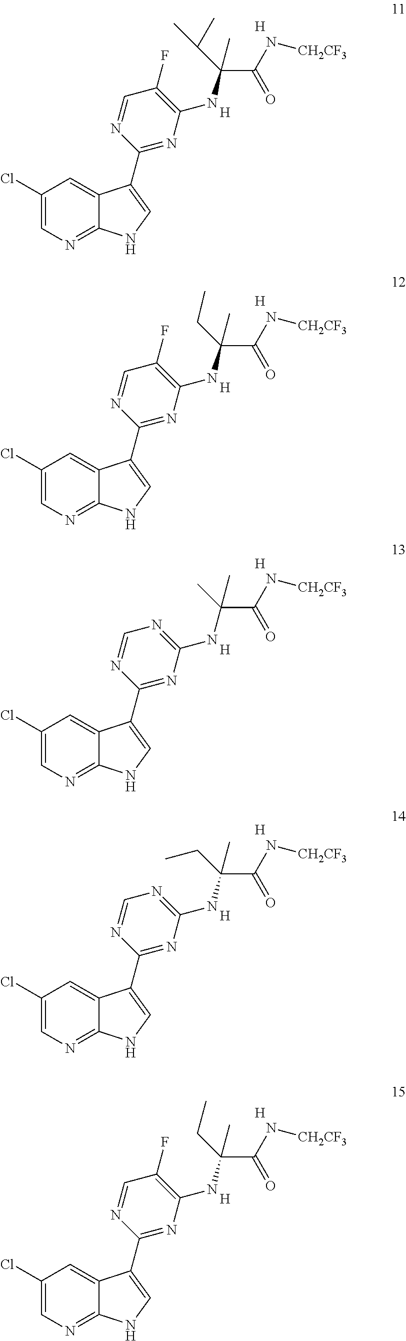

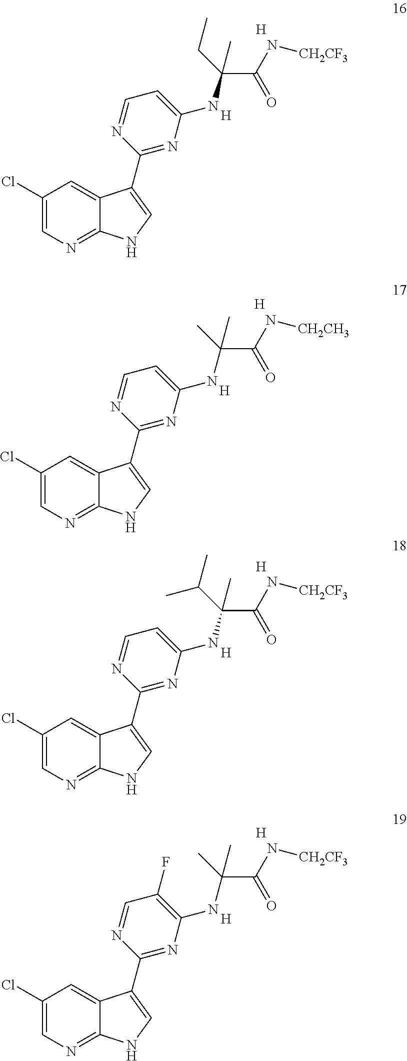

36. The method of any one of claims 22-35, wherein the compound of Formula I is administered at least once per day.

37. The method of any one of claims 22-36, wherein the compound of Formula I is administered at least twice per day.

38. The method of any one of claims 22-37, wherein the compound of Formula I is orally administered to the patient in need thereof.

39. The method of any one of claims 22-38, wherein at least about 100 mg of the compound of Formula I is administered to the patient once per day.

40. The method of any one of claims 22-39, wherein at least about 150 mg of the compound of Formula I is administered to the patient once per day.

41. The method of any one of claims 22-40, wherein at least about 200 mg of the compound of Formula I is administered to the patient once per day.

42. The method of any one of claims 22-38, wherein at least about 100 mg of the compound of Formula I is administered to the patient twice per day.

43. A method for treating or lessening the severity of a disease selected from rheumatoid arthritis, systemic lupus erythematosus, peripheral spondyloarthropathy, axial spondyloarthropathy, ulcerative colitis, Crohn's disease, ankylosing spondylitis, reactive arthritis, Reiter's syndrome, psoriatic arthritis, or any combination thereof comprising administering to a patient in need thereof a chemotherapy agent and a pharmaceutical composition comprising a compound of Formula I 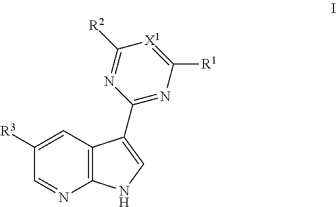
or a pharmaceutically acceptable salt thereof, wherein:
X¹ is N or CR⁴;
R² is H or halo;
R³ is H or halo;
R⁴ is H or halo;
R¹ is 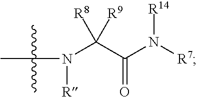
R″ is H or an unsubstituted C₁₋₂ aliphatic;
R⁸ is an unsubstituted C₁₋₄ aliphatic;
R⁹ is an unsubstituted C₁₋₄ aliphatic;
R⁷ is a C₁₋₃ aliphatic optionally substituted with up to 3 occurrences of F; and
R¹⁴ is H or unsubstituted C₁₋₂ alkyl.

44. The method of claim 43, wherein the chemotherapy agent comprises methotrexate, azathioprine, cyclosporine, cyclophosphamide, 6-mercaptopurine, or any combination thereof.

45. The method of either of claim 43 or 44, wherein the chemotherapy agent comprises an injectable formulation or an oral formulation.

46. The method of any one of claims 43-45, wherein the patient is administered from about 5 mg to about 100 mg of the chemotherapy agent per month.

47. The method of any one of claims 43-46, wherein R² is H or F.

48. The method of any one of claims 43-47, wherein R³ is H or Cl.

49. The method of any one of claims 43-48, wherein each of R⁸ and R⁹ is independently selected from methyl, ethyl, propyl, iso-propyl, butyl, or tert-butyl.

50. The method of any one of claims 43-49, wherein each of R⁸ and R⁹ is independently selected from methyl or ethyl.

51. The method of any one of claims 43-50, wherein R¹⁴ is H or methyl.

52. The method of any one of claims 43-51, wherein R⁷ is an unsubstituted C₁₋₃ aliphatic.

53. The method of any one of claims 43-51, wherein R⁷ is a C₁₋₃ aliphatic substituted with 1-3 occurrences of F.

54. The method of any one of claims 43-51, wherein R⁷ is a group selected from —CH₂CH₃, —CH₂CF₃, —CH₂CH₂CH₃, or —CHCH₃CH₃.

55. The method of claim 43, wherein the compound of Formula I is selected from:

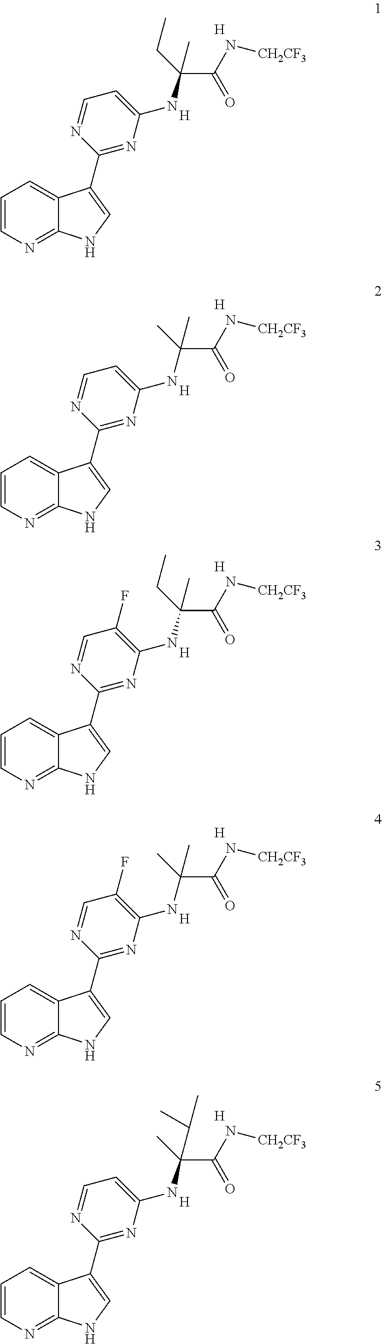

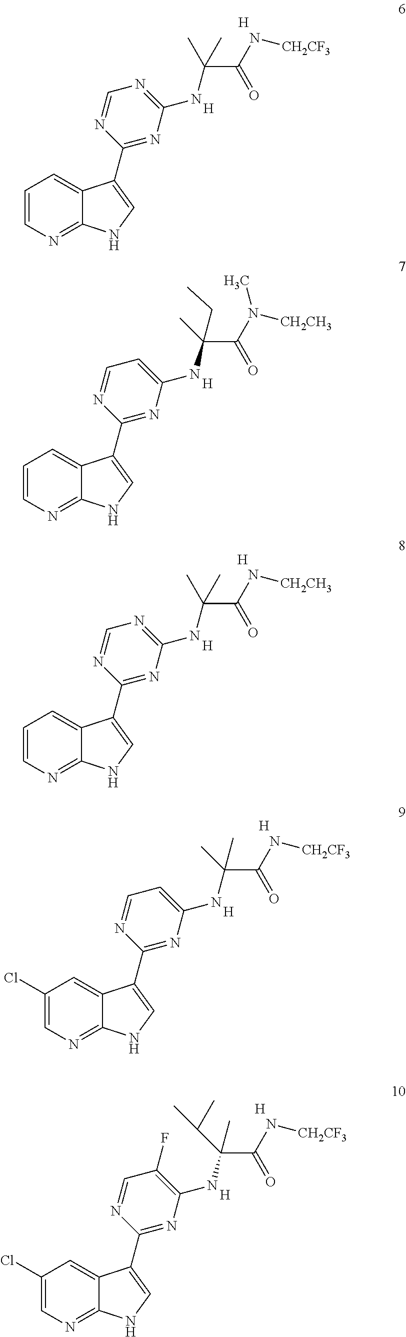

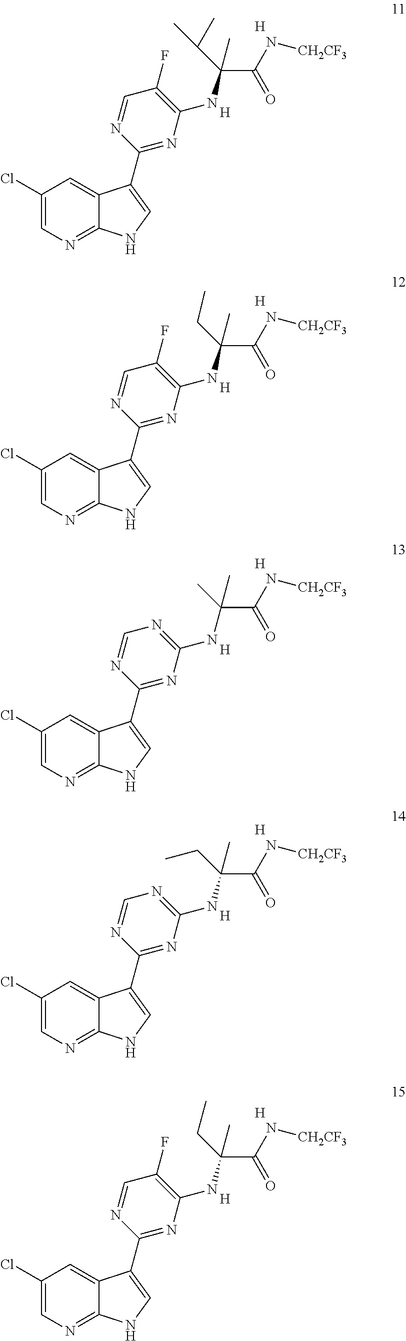

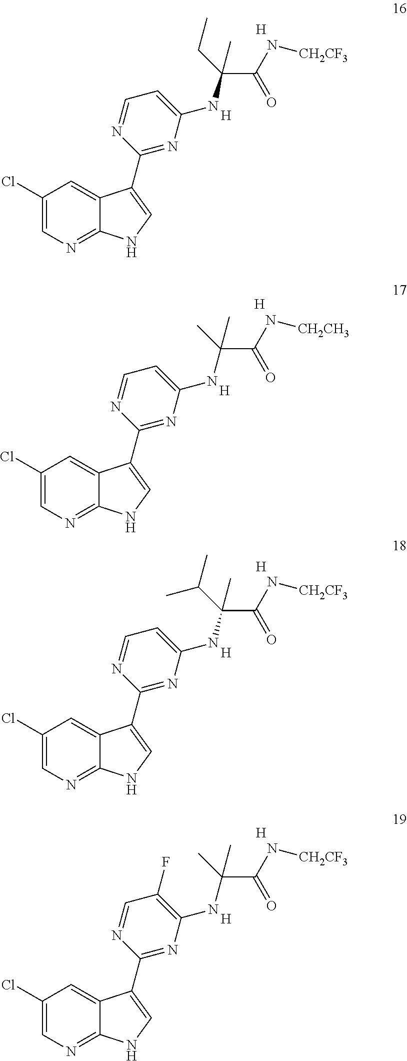

56. The method of any one of claims 43-55, wherein the pharmaceutical composition further comprises a tablet.

57. The method of claim 56, wherein the tablet further comprises a diluent, a binder, a glidant, a disintegrant, a surfactant, a lubricant, or any combination thereof.

58. The method of either of claim 56 or 57, wherein the tablet is administered at least once per day.

59. The method of claim 58, wherein the tablet comprises at least about 10 mg of the compound of Formula I.

60. The method of claim 59, wherein the tablet comprises from about 15 mg to about 100 mg of the compound of Formula I.

61. The method of either of claim 56 or 57, wherein the tablet is administered at least twice per day.

62. The method of claim 61, wherein the tablet comprises at least about 10 mg of the compound of Formula I.

63. The method of claim 62, wherein the tablet comprises from about 15 mg to about 100 mg of the compound of Formula I.

64. The method of either of claim 56 or 57, further comprising administering once per day at least one tablet comprising the pharmaceutical composition.

65. The method of either of claim 56 or 57, further comprising administering twice per day at least one tablet comprising the pharmaceutical composition.

66. The method of either of claim 64 or 65, wherein each tablet further comprises from about 5 mg to about 100 mg of the compound of Formula I.

67. A method for treating or lessening the severity of a disease selected from rheumatoid arthritis, systemic lupus erythematosus, peripheral spondyloarthropathy, axial spondyloarthropathy, ulcerative colitis, Crohn's disease, ankylosing spondylitis, reactive arthritis, Reiter's syndrome, psoriatic arthritis, or any combination thereof comprising administering to a patient in need thereof a compound of Formula I 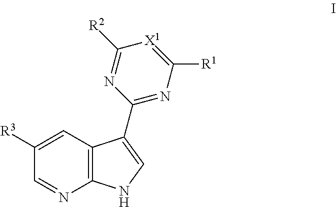
or a pharmaceutically acceptable salt thereof, wherein:
X¹ is N or CR⁴;
R² is H or halo;
R³ is H or halo;
R⁴ is H or halo;
R¹ is 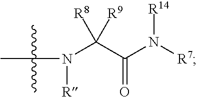
R″ is H or an unsubstituted C₁₋₂ aliphatic;
R⁸ is an unsubstituted C₁₋₄ aliphatic;
R⁹ is an unsubstituted C₁₋₄ aliphatic;
R⁷ is a C₁₋₃ aliphatic optionally substituted with up to 3 occurrences of F; and
R¹⁴ is H or unsubstituted C₁₋₂ alkyl,
or a pharmaceutically acceptable salt thereof wherein:
at least about 100 mg of the compound of Formula I is administered to the patient at least once per day.

68. The method of claim 67, wherein about 100 mg of the compound of formula I is administered to the patient once per day.

69. The method of claim 67, wherein about 150 mg of the compound of formula I is administered to the patient once per day.

70. The method of claim 67, wherein about 200 mg of the compound of formula I is administered to the patient once per day.

71. The method of claim 67, wherein about 100 mg of the compound of formula I is administered to the patient twice per day.

72. The method of any one of claims 67-71, further comprising administering to the patient a chemotherapy agent.

73. The method of any one of claims 67-72, wherein R² is H or F.

74. The method of any one of claims 67-73, wherein R³ is H or Cl.

75. The method of any one of claims 67-74, wherein each of R⁸ and R⁹ is independently selected from methyl, ethyl, propyl, iso-propyl, butyl, or tert-butyl.

76. The method of any one of claims 67-75, wherein each of R⁸ and R⁹ is independently selected from methyl or ethyl.

77. The method of any one of claims 67-76, wherein R¹⁴ is H or methyl.

78. The method of any one of claims 67-77, wherein R⁷ is an unsubstituted C₁₋₃ aliphatic.

79. The method of any one of claims 67-77, wherein R⁷ is a C₁₋₃ aliphatic substituted with 1-3 occurrences of F.

80. The method of any one of claims 67-77, wherein R⁷ is a group selected from —CH₂CH₃, —CH₂CF₃, —CH₂CH₂CH₃, or —CHCH₃CH₃.

81. The method of any one of claims 67-80, wherein the compound of Formula I is selected from:

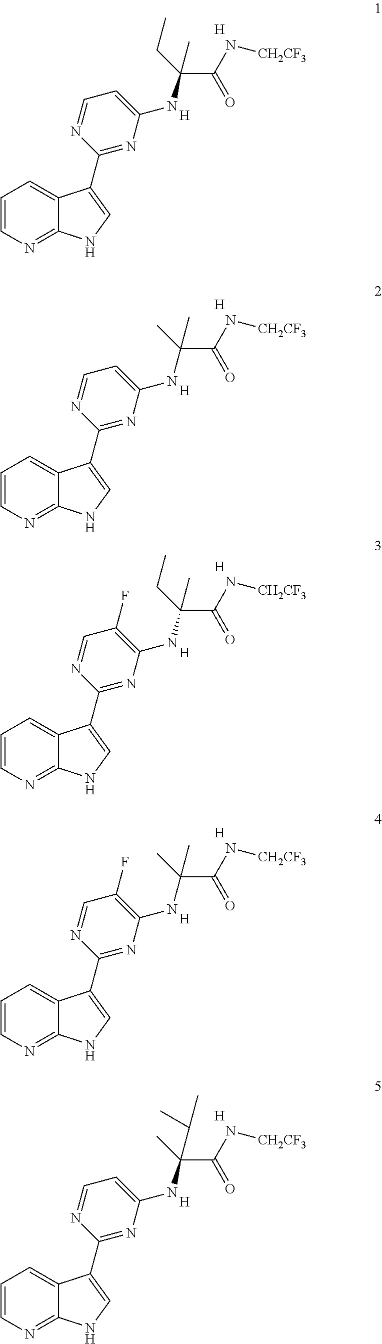

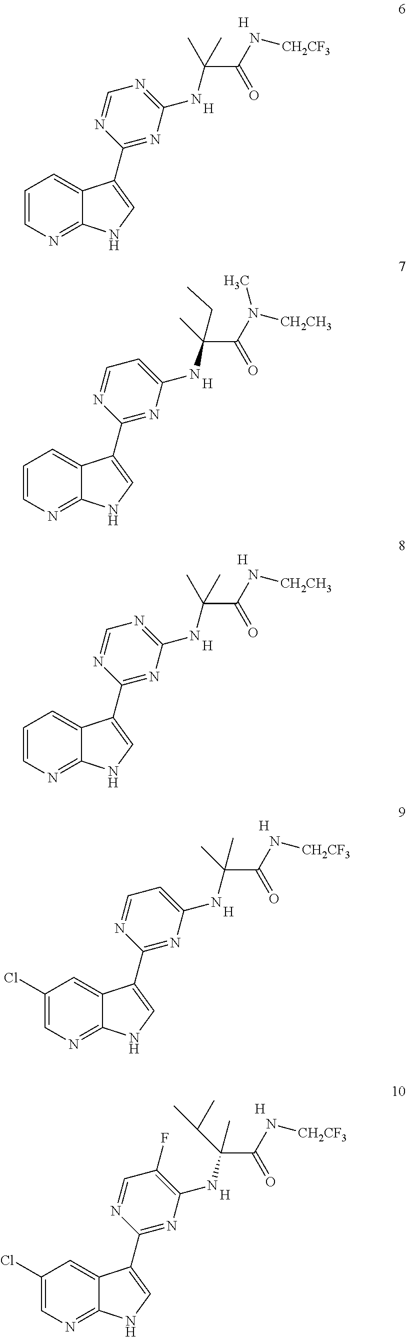

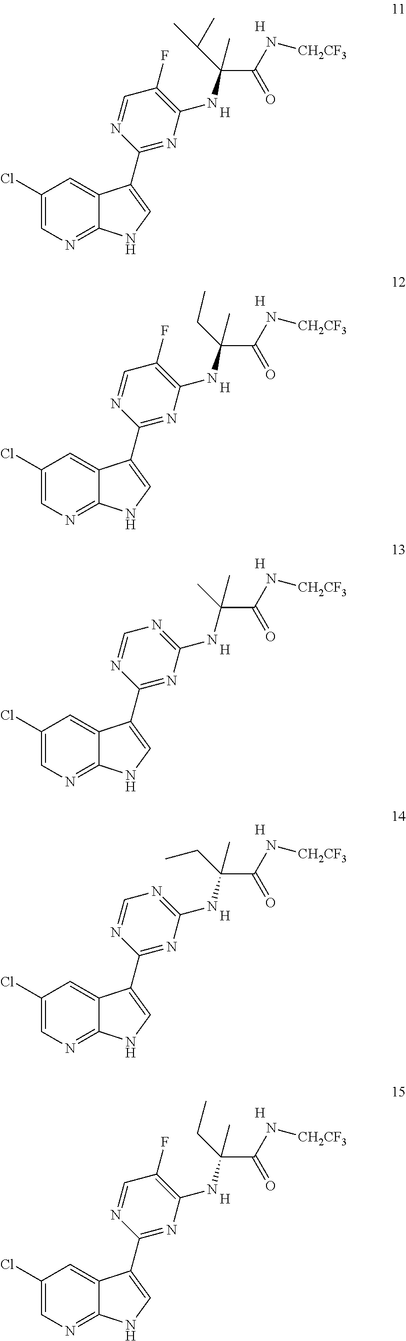

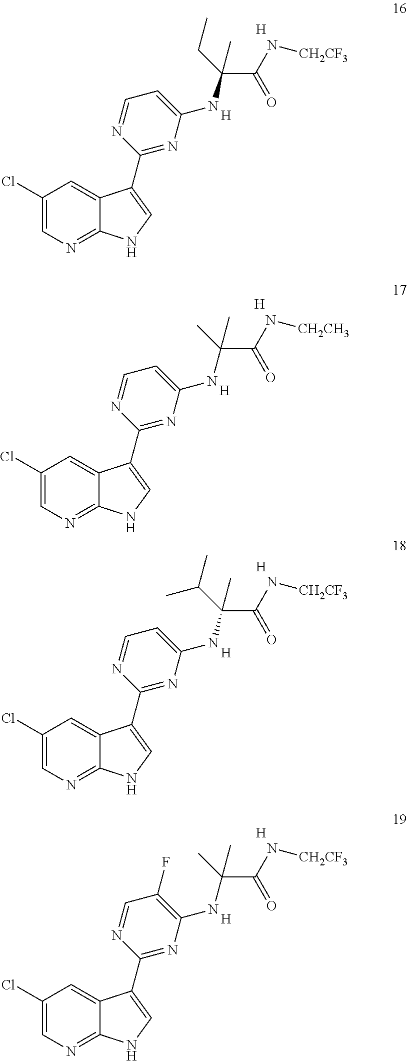

82. A pharmaceutical composition comprising:
a. a compound of Formula I 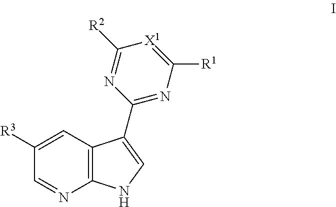
or a pharmaceutically acceptable salt thereof, wherein:
X¹ is N or CR⁴;
R² is H or halo;
R³ is H or halo;
R⁴ is H or halo;
R¹ is 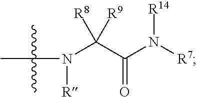
R″ is H or an unsubstituted C₁₋₂ aliphatic;
R⁸ is an unsubstituted C₁₋₄ aliphatic;
R⁹ is an unsubstituted C₁₋₄ aliphatic;
R⁷ is a C₁₋₃ aliphatic optionally substituted with up to 3 occurrences of F; and
R¹⁴ is H or unsubstituted C₁₋₂ alkyl; and
one or more excipients comprising a diluent, a disintegrant, a wetting agent, a binder, a glidant, a lubricant, or any combination thereon, wherein the compound of Formula I has a concentration of from about 25 wt % to about 60 wt % by weight of the composition, and the total concentration for the one or more excipients is from about 40 wt % to about 75 wt % by weight of the composition.

83. The pharmaceutical composition of claim 82, wherein X¹ is N, CH, or CF.

84. The pharmaceutical composition of either of claim 82 or 83, wherein R″ is H or methyl.

85. The pharmaceutical composition of any one of claims 82-84, wherein R² is H or F.

86. The pharmaceutical composition of any one of claims 82-85, wherein each of R⁸ and R⁹ is independently selected from methyl, ethyl, propyl, iso-propyl, butyl, or tert-butyl.

87. The pharmaceutical composition of any one of claims 82-86, wherein each of R⁸ and R⁹ is independently selected from methyl or ethyl.

88. The pharmaceutical composition of any one of claims 82-87, wherein R¹⁴ is H or methyl.

89. The pharmaceutical composition of any one of claims 82-88, wherein R⁷ is an unsubstituted C₁₋₃ aliphatic.

90. The pharmaceutical composition of any one of claims 82-88, wherein R⁷ is a C₁₋₃ aliphatic substituted with 1-3 occurrences of F.

91. The pharmaceutical composition of any one of claims 82-88, wherein R⁷ is a group selected from —CH₂CH₃, —CH₂CF₃, —CH₂CH₂CH₃, or —CHCH₃CH₃.

92. The pharmaceutical composition of any one of claims 82-91, wherein the compound of Formula I is selected from:

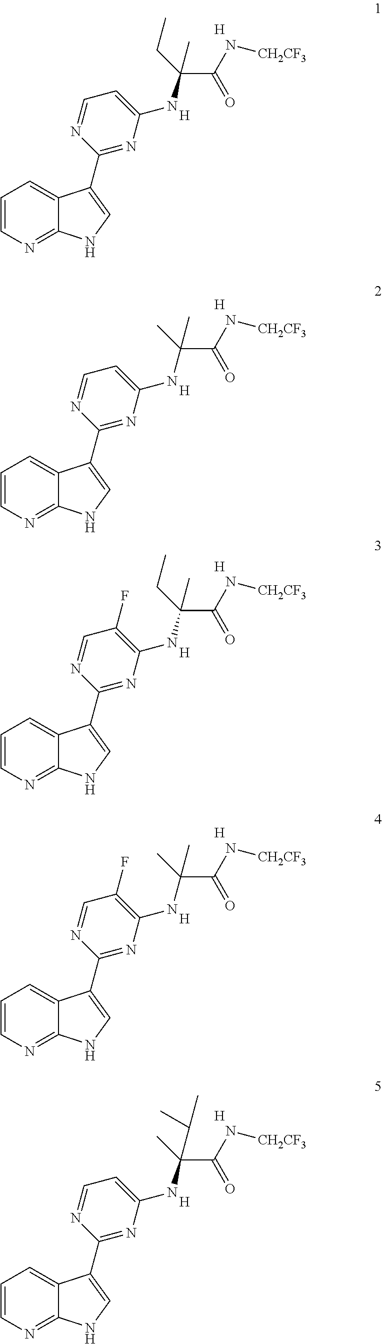

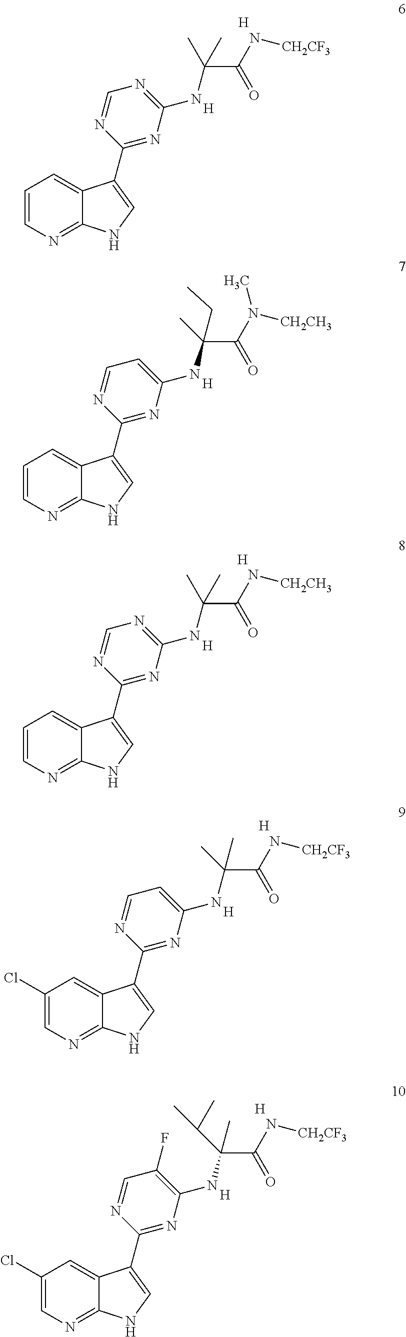

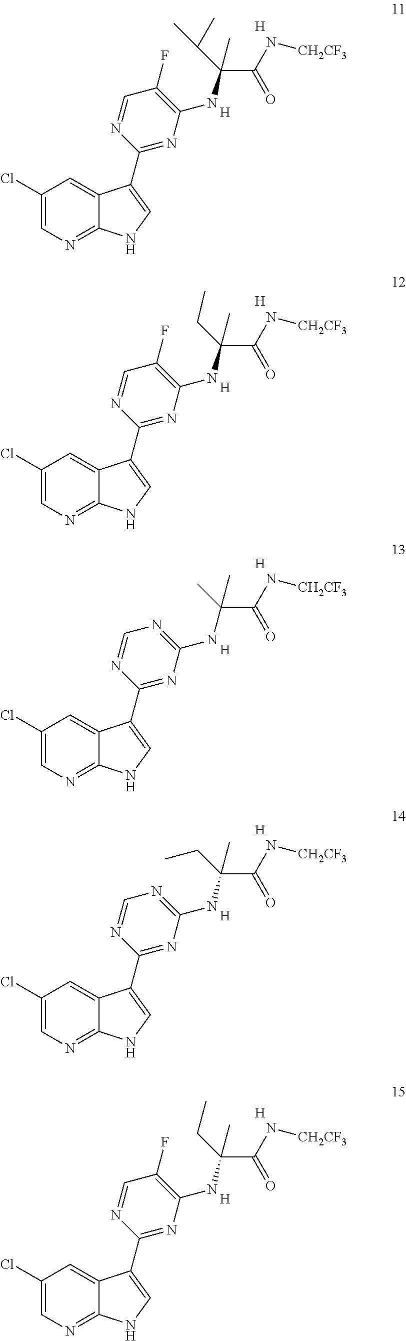

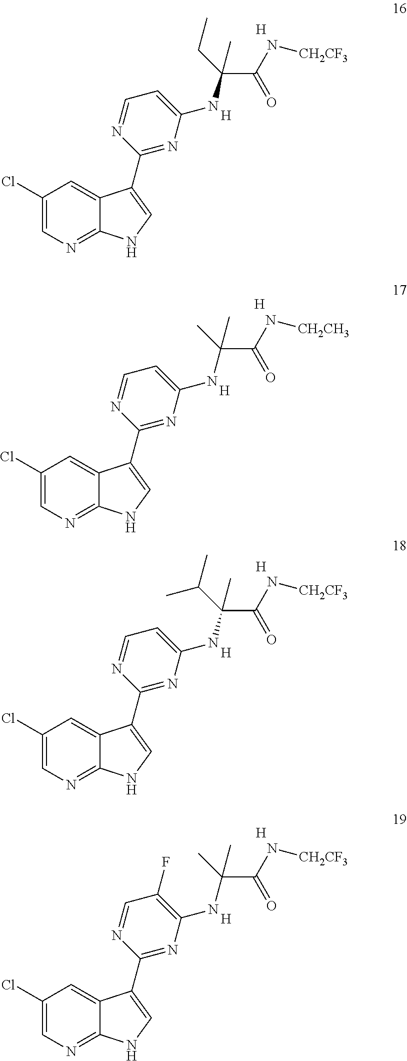

93. The pharmaceutical composition of any one of claims 82-92, further comprising a diluent, and the diluent comprises lactose, sorbitol, cellulose, calcium phosphate, starch, sugar, or any combination thereof.

94. The pharmaceutical composition of claim 93, wherein the diluent comprises lactose and has a concentration of about 10 wt % or greater by weight of the composition.

95. The pharmaceutical composition of any one of claims 82-94, further comprising a disintegrant, and the disintegrant comprises sodium croscarmellose, sodium starch glycolate, or any combination thereof.

96. The pharmaceutical composition of claim 95, wherein the disintegrant comprises sodium croscarmellose and has a concentration of about 10 wt % or less by weight of the composition.

97. The pharmaceutical composition of any one of claims 82-96, further comprising a wetting agent, and the wetting agent comprises sodium lauryl sulfate, sodium stearyl fumarate, polyoxyethylene 20 sorbitan mono-oleate, or any combination thereof.

98. The pharmaceutical composition of claim 97, wherein the wetting agent comprises sodium lauryl sulfate and has a concentration of about 10 wt % or less by weight of the composition.

99. The pharmaceutical composition of any one of claims 82-98, further comprising a binder, and the binder comprises microcrystalline cellulose, dibasic calcium phosphate, sucrose, corn starch, modified cellulose, or any combination thereof.

100. The pharmaceutical composition of claim 99, wherein the binder comprises microcrystalline cellulose and has a concentration of at least about 1 wt % by weight of the composition.

101. The pharmaceutical composition of any one of claims 82-100, further comprising a glidant, and the glidant comprises colloidal silicon dioxide, talc, or any combination thereof.

102. The pharmaceutical composition of claim 101, wherein the glidant comprises colloidal silicon dioxide and has a concentration of 2 wt % or less by weight of the composition.

103. The pharmaceutical composition of any one of claims 82-102, further comprising a lubricant, and the lubricant comprises magnesium stearate, stearic acid, hydrogenated oil, sodium stearyl fumarate, or any combination thereof.

104. The pharmaceutical composition of claim 103, wherein the lubricant comprises magnesium stearate and has a concentration of less than about 2 wt % by weight of the composition.

105. The pharmaceutical composition of any one of claims 82-104, further comprising a colorant.

106. The pharmaceutical composition of any one of claims 82-105, wherein the composition comprises about 25 wt % to about 35 wt % of the compound of Formula I by weight of the composition.

107. The pharmaceutical composition of any one of claims 82-106, wherein the composition comprises from about 45 wt % to about 55 wt % of the compound of Formula I by weight of the composition.

108. The pharmaceutical composition of any one of claims 82-107, wherein the composition comprises a tablet.
